(19) United States
(12) Patent Application Publication
(10) Pub. No.: US 2024/0254177 A1
(43) Pub. Date: Aug. 1, 2024

(54) COMPLEMENT FACTOR H AND Fc BINDING DOMAIN FUSION PROTEINS

(71) Applicant: Alexion Pharmaceuticals, Inc., Boston, MA (US)

(72) Inventors: Krista K. Johnson, Southington, CT (US); Sung-kwon Kim, Cheshire, CT (US); Jeffrey Hunter, Wallingford, CT (US); Christian Cobaugh, Newton Highlands, MA (US)

(73) Assignee: Alexion Pharmaceuticals, Inc., Boston, MA (US)

(21) Appl. No.: 17/267,164

(22) PCT Filed: Aug. 22, 2019
(86) PCT No.: PCT/US2019/047786

§ 371 Date: Feb. 9, 2021

Related U.S. Application Data
(60) Provisional application No. 62/721,348, filed on Aug. 22, 2018.

(51) Int. Cl. C07K 14/47; A61K 38/00; A61K 39/00; A61P 13/12

(52) U.S. Cl. CPC C07K 14/4703; A61P 13/12; A61K 38/00; A61K 2039/505; C07K 2319/30; C07K 2319/50

(57) ABSTRACT

Described herein are fusion proteins that include a fragment of factor H and an Fc receptor binding domain, as well as the use of such proteins in methods of treatment for diseases mediated by alternative complement pathway dysregulation.

### SEQUENCE LISTING

This application contains a Sequence Listing which has been submitted electronically in ASCII format and is hereby incorporated by reference in its entirety. Said ASCII copy, created on Aug. 22, 2019, is named 51196-004WO2_Sequence_Listing_08.22.19_ST25 and is 83,703 bytes in size.

### BACKGROUND

The complement system plays a central role in the clearance of immune complexes and in immune responses to infectious agents, foreign antigens, virus-infected cells, and tumor cells. Complement activation occurs primarily by three pathways: the classical pathway, the lectin pathway, and the alternative pathway. The alternative pathway of complement activation is in a constant state of low-level activation. Uncontrolled activation or insufficient regulation of the alternative complement pathway can lead to systemic inflammation, cellular injury, and tissue damage. Thus, the alternative complement pathway has been implicated in the pathogenesis of a number of diverse diseases. Inhibition or modulation of alternative complement pathway activity has been recognized as a promising therapeutic strategy. The number of treatment options available for these diseases are limited. Thus, developing innovative strategies to treat diseases associated with alternative complement pathway dysregulation is a significant unmet need.

### SUMMARY

Described herein are engineered fusion proteins that include complement factor H (FH) or a functional fragment of FH fused to Fc receptor binding domains, such as monoclonal antibodies, antibody derivatives, and fragments thereof. The fusion proteins can be used to treat subjects with diseases associated with alternative complement pathway dysregulation.

In one aspect, the disclosure is directed to a fusion protein including a first moiety including complement factor H (FH) or a functional fragment of FH and a second moiety including an Fc receptor binding domain. In some embodiments, the first moiety is fused to the second moiety by a linker. In some embodiments, the first moiety is fused to the second moiety by a covalent bond.

In one aspect, the disclosure is directed to a fusion protein consisting of a first moiety having the first four N-terminal short consensus repeat (SCR) domains of complement factor H (FH) fused by a linker to a second moiety having an Fc receptor binding domain.

In one aspect, the disclosure is directed to a fusion protein consisting of a first moiety having the first five N-terminal short consensus repeat (SCR) domains of complement factor H (FH) fused by a linker to a second moiety having an Fc receptor binding domain.

In some embodiments, the first moiety consists of, from N-terminus to C-terminus, FH SCR domains 1, 2, 3 and 4 (e.g., a fragment of factor H of SEQ ID NO: 91) or FH SCR domains 1, 2, 3, 4 and 5 (e.g., a fragment of factor H of SEQ ID NO: 92). In some embodiments of any of the above aspects, the first moiety consists of, from N-terminus to C-terminus, FH SCR domains 4, 3, 2 and 1 or FH SCR domains 5, 4, 3, 2 and 1.

In some embodiments, the linker is a cleavable linker, such as an enzymatically cleavable linker. In some embodiments, the linker is cleavable by trypsin, Human Rhinovirus 3C Protease (3C), enterokinase (Ekt), Factor Xa (FXa), Tobacco Etch Virus protease (TEV), or thrombin (Thr).

In some embodiments, the linker includes an amino acid sequence. In some embodiments, the linker is a polymeric or oligomeric glycine linker. In other embodiments the linker includes a lysine at the N-terminus, the C-terminus, or both the N- and the C-termini. In some embodiments, the linker is selected from the group consisting of: (G₄A)₂G₃AG₄S (SEQ ID NO: 109), G₄AG₃AG₄S (SEQ ID NO:108), (G₄A)₂G₄S (SEQ ID NO: 7), GGGGAGGGGAGGGGS (SEQ ID NO: 7), GGGGSGGGGSGGGGS (SEQ ID NO: 11), G₄S (SEQ ID NO: 9), (G₄S)₂ (SEQ ID NO: 10), (G₄S)₃ (SEQ ID NO: 11), (G₄S)₄ (SEQ ID NO: 12), (G₄S)₅ (SEQ ID NO: 13), (G₄S)₆ (SEQ ID NO: 14), (EAAAK)₃ (SEQ ID NO: 15), PAPAP (SEQ ID NO: 16), G₄SPAPAP (SEQ ID NO: 17), PAPAPG₄S (SEQ ID NO: 18), GSTSGKSSEGKG (SEQ ID NO: 19), (GGGDS)₂ (SEQ ID NO: 20), (GGGES)₂ (SEQ ID NO: 21), GGGDSGGGGS (SEQ ID NO: 22), GGGASGGGGS (SEQ ID NO: 23), GGGESGGGGS (SEQ ID NO: 24), ASTKGP (SEQ ID NO: 25), ASTKGPSVFPLAP (SEQ ID NO: 26), G₃P (SEQ ID NO: 27), G₇P (SEQ ID NO: 28), PAPNLLGGP (SEQ ID NO: 29), G₆ (SEQ ID NO: 30), G₁₂ (SEQ ID NO: 31), APELPGGP (SEQ ID NO: 32), SEPQPQPG (SEQ ID NO: 33), (G₃S₂)₃ (SEQ ID NO: 34), GGGGGGGGGSGGGS (SEQ ID NO: 35), GGGGSGGGGGGGGGS (SEQ ID NO: 36), (GGSSS)₃ (SEQ ID NO: 37), (GS₄)₃ (SEQ ID NO: 38), G₄A(G₄S)₂ (SEQ ID NO: 39), G₄SG₄AG₄ (SEQ ID NO: 40), G₃AS(G₄S)₂ (SEQ ID NO: 41), G₄SG₃ASG₄S (SEQ ID NO: 42), G₄SAG₃SG₄S (SEQ ID NO: 43), (G₄S)₂AG₃S (SEQ ID NO: 44), G₄SAG₃SAG₃S (SEQ ID NO: 45), G₄D(G₄S)₂ (SEQ ID NO: 46), G₄SG₄DG₄S (SEQ ID NO: 47), (G₄D)₂G₄S (SEQ ID NO: 48), G₄E(G₄S)₂ (SEQ ID NO: 49), G₄SG₄EG₄S (SEQ ID NO: 50), (G₄E)₂G₄S (SEQ ID NO: 51), K(G₄A)₂G₃AG₄SK (SEQ ID NO:110), R(G₄A)₂G₃AG₄SR (SEQ ID NO:111), K(G₄A)₂G₃AG₄SR (SEQ ID NO:112), R(G₄A)₂G₃AG₄SK (SEQ ID NO:113), K(G₄A)₂G₄SK (SEQ ID NO:114), K(G₄A)₂G₄SR (SEQ ID NO:115), R(G₄A)₂G₄SK (SEQ ID NO:116), and R(G₄A)₂G₄SR (SEQ ID NO:117), and G₄SG₄AG₄S (SEQ ID NO:118).

In particular embodiments, the first moiety is fused to the N-terminus of the second moiety. In other embodiments, the first moiety is fused to the C-terminus of the second moiety.

In some embodiments, the Fc receptor binding domain is an antibody or an Fc receptor binding fragment thereof. In some embodiments, the antibody is a monoclonal IgG antibody. In a particular embodiment, the monoclonal IgG antibody is a human monoclonal IgG antibody. In other embodiments, the Fc receptor binding fragment is a fragment crystallizable (Fc) domain. In particular embodiments, the Fc domain is a human IgG-Fc domain.

In particular embodiments, the fusion protein forms a dimer.

In some embodiments, the fusion protein has an increased half-life compared to factor H, or a fragment thereof, that is not fused to an Fc receptor binding domain.

In some embodiments, the fusion protein includes a tag, e.g., a purification tag. In some embodiments, the tag is a poly histidine tag, e.g., a 6-histidine tag, or a glutathione S-transferase (GST) tag.

In one aspect, the fusion protein has an amino acid sequence of SEQ ID NO: 98, or is variant thereof having up to 10 amino acid substitutions, additions, or deletions. In some embodiments, the fusion protein is a variant of SEQ ID NO: 98 having 9, 8, 7, 6, 5, 4, 3, 2, or 1 amino acid substitution(s), addition(s), or deletion(s).

In one aspect, the fusion protein has an amino acid sequence of SEQ ID NO: 99, or is variant thereof having up to 10 amino acid substitutions, additions, or deletions. In some embodiments, the fusion protein is a variant of SEQ ID NO:99 having 9, 8, 7, 6, 5, 4, 3, 2, or 1 amino acid substitution(s), addition(s), or deletion(s).

In one aspect, the fusion protein has an amino acid sequence of SEQ ID NO: 100, or is a variant thereof having up to 10 amino acid substitutions, additions, or deletions. In some embodiments, the fusion protein is a variant of SEQ ID NO:100 having 9, 8, 7, 6, 5, 4, 3, 2, or 1 amino acid substitution(s), addition(s), or deletion(s).

In one aspect, the fusion protein has an amino acid sequence of SEQ ID NO: 101, or is a variant thereof having up to 10 amino acid substitutions, additions, or deletions. In some embodiments, the fusion protein is a variant of SEQ ID NO:101 having 9, 8, 7, 6, 5, 4, 3, 2, or 1 amino acid substitution(s), addition(s), or deletion(s).

In one aspect, the fusion protein has an amino acid sequence of SEQ ID NO: 102, or is a variant thereof having up to 10 amino acid substitutions, additions, or deletions. In some embodiments, the fusion protein is a variant of SEQ ID NO:102 having 9, 8, 7, 6, 5, 4, 3, 2, or 1 amino acid substitution(s), addition(s), or deletion(s).

In one aspect, the fusion protein has an amino acid sequence of SEQ ID NO: 103, or is a variant thereof having up to 10 amino acid substitutions, additions, or deletions. In some embodiments, the fusion protein is a variant of SEQ ID NO:103 having 9, 8, 7, 6, 5, 4, 3, 2, or 1 amino acid substitution(s), addition(s), or deletion(s).

In one aspect, the fusion protein has an amino acid sequence of SEQ ID NO: 104, or is a variant thereof having up to 10 amino acid substitutions, additions, or deletions. In some embodiments, the fusion protein is a variant of SEQ ID NO:104 having 9, 8, 7, 6, 5, 4, 3, 2, or 1 amino acid substitution(s), addition(s), or deletion(s).

In one aspect, the fusion protein has an amino acid sequence of SEQ ID NO: 105, or is a variant thereof having up to 10 amino acid substitutions, additions, or deletions. In some embodiments, the fusion protein is a variant of SEQ ID NO:105 having 9, 8, 7, 6, 5, 4, 3, 2, or 1 amino acid substitution(s), addition(s), or deletion(s).

In one aspect, the fusion protein has an amino acid sequence of SEQ ID NO: 106, or is a variant thereof having up to 10 amino acid substitutions, additions, or deletions. In some embodiments, the fusion protein is a variant of SEQ ID NO:106 having 9, 8, 7, 6, 5, 4, 3, 2, or 1 amino acid substitution(s), addition(s), or deletion(s).

In one aspect, the fusion protein has an amino acid sequence of SEQ ID NO: 107, or is a variant thereof having up to 10 amino acid substitutions, additions, or deletions. In some embodiments, the fusion protein is a variant of SEQ ID NO:107 having 9, 8, 7, 6, 5, 4, 3, 2, or 1 amino acid substitution(s), addition(s), or deletion(s).

In one aspect, the fusion protein has an amino acid sequence having at least 85% sequence identify to SEQ ID NO:98. In some embodiments, the fusion protein has at least 90%, 95%, 98%, or 99% sequence identify to SEQ ID NO:98.

In one aspect, the fusion protein has an amino acid sequence having at least 85% sequence identify to SEQ ID NO:99. In some embodiments, the fusion protein has at least 90%, 95%, 98%, or 99% sequence identify to SEQ ID NO:99.

In one aspect, the fusion protein has an amino acid sequence having at least 85% sequence identify to SEQ ID NO:100. In some embodiments, the fusion protein has at least 90%, 95%, 98%, or 99% sequence identify to SEQ ID NO:100.

In one aspect, the fusion protein has an amino acid sequence having at least 85% sequence identify to SEQ ID NO:101. In some embodiments, the fusion protein has at least 90%, 95%, 98%, or 99% sequence identify to SEQ ID NO:101.

In one aspect, the fusion protein has an amino acid sequence having at least 85% sequence identify to SEQ ID NO:102. In some embodiments, the fusion protein has at least 90%, 95%, 98%, or 99% sequence identify to SEQ ID NO:102.

In one aspect, the fusion protein has an amino acid sequence having at least 85% sequence identify to SEQ ID NO:103. In some embodiments, the fusion protein has at least 90%, 95%, 98%, or 99% sequence identify to SEQ ID NO:103.

In one aspect, the fusion protein has an amino acid sequence having at least 85% sequence identify to SEQ ID NO:104. In some embodiments, the fusion protein has at least 90%, 95%, 98%, or 99% sequence identify to SEQ ID NO: 104.

In one aspect, the fusion protein has an amino acid sequence having at least 85% sequence identify to SEQ ID NO:105. In some embodiments, the fusion protein has at least 90%, 95%, 98%, or 99% sequence identify to SEQ ID NO:105.

In one aspect, the fusion protein has an amino acid sequence having at least 85% sequence identify to SEQ ID NO:106. In some embodiments, the fusion protein has at least 90%, 95%, 98%, or 99% sequence identify to SEQ ID NO: 106.

In one aspect, the fusion protein has an amino acid sequence having at least 85% sequence identify to SEQ ID NO:107. In some embodiments, the fusion protein has at least 90%, 95%, 98%, or 99% sequence identify to SEQ ID NO: 107.

In one aspect, the disclosure is directed to a nucleic acid or polynucleotide encoding a fusion protein described herein.

In one aspect, the disclosure is directed to a vector including the nucleic acid encoding a fusion protein described herein.

In one aspect, the disclosure is directed to a host cell including the nucleic acid or vector encoding a fusion protein described herein.

In one aspect, the disclosure is directed to a pharmaceutical composition including a fusion protein described herein.

In one aspect, the disclosure is directed to a method of treating a disease mediated by alternative complement pathway dysregulation including administering an effective amount of a pharmaceutical composition including a fusion protein described herein to a subject in need thereof. In some embodiments, the fusion protein has the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107. In one aspect, the disclosure is directed to a method of treating a disease mediated by alternative complement pathway dysregulation including administering an effective amount of a polynucleotide encoding a fusion protein described herein to a subject in need thereof.

In one aspect, the disclosure is directed to a method of treating a disease mediated by alternative complement pathway dysregulation including administering an effective amount of a host cell including a nucleic acid encoding a fusion protein described herein to a subject in need thereof.

In a particular embodiment, the disease is paroxysmal nocturnal hemoglobinuria (PNH). In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat PNH. In a particular embodiment, the disease is atypical hemolytic uremic syndrome (aHUS). In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat aHUS. In a particular embodiment, the disease is IgA nephropathy. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat IgA nephropathy. In a particular embodiment, the disease is lupus nephritis. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat lupus nephritis. In a particular embodiment, the disease is C3 glomerulopathy (C3G). In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat C3G. In a particular embodiment, the disease is dermatomyositis. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat dermatomyositis. In a particular embodiment, the disease is systemic sclerosis. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat systemic sclerosis. In a particular embodiment, the disease is demyelinating polyneuropathy. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat demyelinating polyneuropathy. In a particular embodiment, the disease is pemphigus. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat pemphigus. In a particular embodiment, the disease is dense deposit disease (DDD). In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat DDD. In a particular embodiment, the disease is age related macular degeneration (AMD). In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat AMD. In a particular embodiment, the disease is thrombic thrombocytopenia purpura (TTP). In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat TTP. In a particular embodiment, the disease is systemic lupus erythematosus. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat lupus erythematosus. In a particular embodiment, the disease is rheumatoid arthritis (RA). In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat RA. In a particular embodiment, the disease is antiphospholipid (aPL) Ab syndrome. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat aPL Ab syndrome. In a particular embodiment, the disease is glomerulonephritis. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat glomerulonephritis. In a particular embodiment, the disease is inflammation. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat inflammation. In a particular embodiment, the disease is rejection of a transplanted organ. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used in connection with rejection of a transplanted organ. In a particular embodiment, the disease is intestinal and renal I/R injury. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat intestinal and renal I/R injury. In a particular embodiment, the disease is asthma. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat asthma. In a particular embodiment, the disease is spontaneous fetal loss. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat spontaneous fetal loss. In a particular embodiment, the disease is hemolytic uremic syndrome (HUS). In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat HUS. In a particular embodiment, the disease is ANCA-associated vasculitis. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat ANCA-associated vasculitis. In a particular embodiment, the disease is traumatic brain injury (TBI). In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat TBI. In a particular embodiment, the disease is multiple sclerosis (MS). In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat MS. In a particular embodiment, the disease is ischemia-reperfusion injury. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat ischemia-reperfusion injury. In a particular embodiment, the disease is preeclampsia. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat preeclampsia. In a particular embodiment, the disease is EBA. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat EBA. In a particular embodiment, the disease is lupus nephritis. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat lupus nephritis. In a particular embodiment, the disease is membranous nephropathy. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat membranous nephropathy. In a particular embodiment, the disease is focal segmental glomerular sclerosis (FSGS). In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat focal segmental glomerular sclerosis (FSGS). In a particular embodiment, the disease is bullous pemphigoid. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat bulbous pemphigoid. In a particular embodiment, the disease is epidermolysis bullosa acquisita. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat epidermolysis bullosa acquisita. In a particular embodiment, the disease is hypocomplementemic urticarial vasculitis. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat hypocomplementemic urticarial vasculitis. In a particular embodiment, the disease is immune complex small vessel vasculitis. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat immune complex small vessel vasculitis. In a particular embodiment, the disease is an autoimmune necrotizing myopathy. In a particular embodiment, a fusion protein having the amino acid sequence of SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, or SEQ ID NO:107 is used to treat autoimmune necrotizing myopathy.

In some embodiments, the subject is a mammal. In a particular embodiment, the mammal is a human.

Excluded from this disclosure is a construct consisting of CR2 SCR 1-4 directly fused to FH SCR 1-5 (CR2[1-4]-FH[1-5]), as described in WO 2007/149567.

### BRIEF DESCRIPTION OF THE DRAWINGS

FIG. 1 is a schematic diagram illustrating exemplary fusion proteins containing a fragment of factor H (SCRs 1-5) and a non-targeting monoclonal IgG antibody (referred to herein as mAb-FH₁₋₅) or a fragment of factor H (SCRs 1-5) and an anti-properdin antibody (referred to herein as Anti-Properdin FH₁₋₅ or Anti-P-FH₁₋₅).

FIG. 2 is an immunoblot examining the pharmacokinetic profile of the non-targeting mAb-FH₁₋₅, anti-P-FH₁₋₅, anti-P control mAb, or mAb control IgG in FH^{-/-} mice following a single injection.

FIG. 3A is a graph showing plasma C3 levels in FH^{-/-} mice at 24 hours and 96 hours following a single injection of non-targeting mAb-FH₁₋₅, anti-P-FH₁₋₅, anti-P control mAb, or mAb control IgG.

FIG. 3B is a graph showing plasma C5 levels in FH^{-/-} mice at 24 hours and 96 hours following a single injection of non-targeting mAb-FH₁₋₅, anti-P-FH₁₋₅, anti-P control mAb, or mAb control IgG.

FIG. 3C is an immunoblot showing plasma factor B (FB) levels in FH^{-/-} mice at 24 hours and 96 hours following a single injection of non-targeting mAb-FH₁₋₅, anti-P-FH₁₋₅, anti-P control mAb, or mAb control IgG.

FIG. 3D is a series of graphs showing plasma C3c and C3d levels in FH^{-/-} mice at 24 hours and 96 hours following a single injection of non-targeting mAb-FH₁₋₅, anti-P-FH₁₋₅, anti-P control mAb, or mAb control IgG.

FIG. 3E is a series of immunofluorescence images showing glomerular iC3b/C3b/C3c and C3d levels following a single injection of non-targeting mAb-FH₁₋₅, anti-P-FH₁₋₅, anti-P control mAb, or mAb control IgG.

FIG. 4 is a graph showing plasma properdin levels in FH^{-/-} mice at 24 hours and 96 hours following a single injection of non-targeting mAb-FH₁₋₅, anti-P-FH₁₋₅, anti-P control mAb, or mAb control IgG. P values derived from ANOVA with Bonferroni correction; **** P<0.0001, *** P=0.0004, ** P=0.0071.

FIG. 5A is a series of immunofluorescence images showing mesangial properdin reactivity in FH^{-/-} mice following a single injection of non-targeting mAB-FH₁₋₅, anti-P-FH₁₋₅, anti-P control mAb, or mAb control IgG.

FIG. 5B is a series of immunofluorescence images showing mesangial IgG reactivity in FH^{-/-} mice following a single injection of non-targeting mAb-FH₁₋₅, anti-P-FH₁₋₅, anti-P control mAb, or mAb control IgG.

FIG. 5C is a series of immunofluorescence images showing mesangial factor H (FH) reactivity in FH^{-/-} mice following a single injection of non-targeting mAb-FH₁₋₅, anti-P-FH₁₋₅, anti-P control mAb, or mAb control IgG.

FIG. 5D is a series of immunofluorescence images showing mesangial factor H-related protein (FHR) reactivity in FH^{-/-} mice following a single injection of non-targeting mAb-FH₁₋₅, anti-P-FH₁₋₅, anti-P control mAb, or mAb control IgG.

FIG. 6A is schematic of an experimental overview of the properdin depleted FH^{-/-} model to examine the effect of properdin depletion on anti-P-FH₁₋₅ deposition.

FIG. 6B and FIG. 6C are a set of graphs showing plasma properdin levels in properdin depleted (FIG. 6B) or control (FIG. 6C) FH^{-/-} mice at 24 hours and 96 hours following a single injection of non-targeting mAb-FH₁₋₅, anti-P-FH₁₋₅, or mAb control IgG.

FIG. 6D is a series of immunofluorescence images showing mesangial IgG, FH/FHR, and properdin reactivity in properdin depleted (right panel) or control (left panel) FH^{-/-} mice following a single injection of non-targeting mAb-FH₁₋₅, anti-P-FH₁₋₅, or mAb control IgG.

FIG. 7 shows a timeline for assaying for the effect of Fc-FH fusion in an in vivo mouse model of human membranoproliferative glomerulonephritis (MPGN).

FIG. 8A, FIG. 8B, and FIG. 8C are a series of graphs showing that non-targeting mAb-FH₁₋₅, which contains a non-targeting mAb fused to FH SCR1-5, treatment significantly attenuates glomerular injury and improves kidney function in vivo.

FIG. 9 is a series of images showing that non-targeting mAb-FH₁₋₅ treatment improves clearance of C3 in vivo. In each panel, the letter “M” followed by a number refers to the mouse number.

FIG. 10 is a series of images showing that non-targeting mAb-FH₁₋₅ treatment attenuates C9 (MAC) deposit in vivo. In each panel, the letter “M” followed by a number refers to the mouse number.

FIG. 11A and FIG. 11B is a series of graphs showing non-targeting mAb-FH₁₋₅ treatment significantly attenuates plasma C3 (FIG. 11A) and C5 (FIG. 11B) levels in vivo.

FIG. 12 is a schematic showing an exemplary timeline for assaying for the effect of Fc-FH fusion in an in vivo mouse model of human atypical hemolytic uremic syndrome (aHUS).

FIG. 13 is a series of graphs showing that treatment with non-targeting mAb-FH₁₋₅ improves survival and increases body weight in vivo. “A” refers to Compound A (anti-properdin mAb), “B” refers to Compound B (non-targeting mAb-FH₁₋₅ fusion), and “C” refers to Compound C (control mAb).

FIG. 14 is a series of graphs showing that treatment with non-targeting mAb-FH₁₋₅ (Compound B) but not control antibody (Compound C) improves platelet count in vivo. Compound A is an anti-properdin mAb control.

FIG. 15 is a series of graphs showing that treatment with non-targeting mAb-FH₁₋₅ (Compound B) but not control antibody (Compound C) improves hemoglobin count in vivo. Compound A is an anti-properdin mAb control.

FIG. 16 is a series of graphs showing that treatment with non-targeting mAb-FH₁₋₅ (Compound B) but not control antibody (Compound C) reduces reticulocyte count in vivo. Compound A is an anti-properdin mAb.

FIG. 17 is a series of images showing that treatment with non-targeting mAb-FH₁₋₅ (Compound B) but not control antibody (Compound C) reduces mesangial expansion and endothelial swelling with narrowing capillary lumen. Compound A is an anti-properdin mAb control.

FIG. 18 is a series of graphs showing that treatment with non-targeting mAb-FH₁₋₅ (Compound B) but not control antibody (Compound C) reduces hematuria, proteinuria, and also blood urea nitrogen (BUN) in vivo. Compound A is an anti-properdin mAb control.

FIG. 19 is a graph and a series of images showing that treatment with non-targeting mAb-FH₁₋₅ (Compound B) but not control antibody (Compound C) reduces C3 levels in kidney. “W” refers to wild-type, “A” refers to Compound A, “B” refers to Compound B, and “C” refers to Compound C.

FIG. 20 is a graph and a series of images showing that treatment with non-targeting mAb-FH₁₋₅ (Compound B) but not control antibody (Compound C) reduces fibrin levels in kidney. “A” refers to Compound A, which is an anti-properdin mAb control, “B” refers to Compound B, and “C” refers to Compound C.

FIG. 21 is a series of images showing that treatment with non-targeting mAb-FH₁₋₅ (Compound B) but not control antibody (Compound C) reduces incidence of large thrombi in kidneys and liver of FH^{R/R} mice. Thrombi are indicated by arrows. Compound A is an anti-properdin mAb control.

FIG. 22 is a graph showing results on the ex vivo effect of the Fc-FH₁₋₅ fusion protein against hemolysis.

FIG. 23 is a graph showing results on the in vitro assessment of the Fc-FH₁₋₅ fusion protein against hemolysis in 20% normal mouse serum.

FIG. 24 is a graph showing that anti-properdin mAb fused to FH shows stronger inhibitory effect against hemolysis compared to that anti-properdin mAb alone. The experiment was performed in 20% normal mouse serum for 30 minutes at 37ºC.

FIG. 25 is a graph showing the inhibitory effect of various fusion constructs of Fc and FH compared to control mAb and a control CR2-FH fusion against hemolysis. The experiment was performed in 20% normal mouse serum for 30 minutes at 37° C.

FIG. 26 is a graph showing inhibition of human alternative complement pathway hemolysis by non-targeted FH-Fc fusion proteins.

FIG. 27 is a graph showing activity of Compound 1 against alternative complement pathway hemolysis in vitro. The experiment was performed in 22% normal mouse serum for 30 minutes at 37ºC.

FIG. 28A and FIG. 28B are graphs showing the in vivo effect of the Fc-FH₁₋₄ fusion protein against complement alternative pathway hemolysis. FIG. 28A shows inhibitory activity a single 25 mg/kg dose of Compound 1 (mouse IgG1 Fc fused to mouse short consensus repeat domains (SCR) 1-4 of factor H). The experiment was performed in 15.4% normal mouse serum for 60 minutes at 37ºC. FIG. 28B shows in vivo inhibitory effect (mean±STD) of a single administration of non-targeting mAb-FH₁₋₅ fusion proteins (triangle=6 mg/kg; square=18 mg/kg; and circle=56 mg/kg) in C57Bl/6 male mice.

FIG. 29 is a bar graph showing reduction in the keyhole limpet haemocyanin (KLH)-specific IgM response on day 7 in immunized animals administered cyclophosphamide, Compound 5, and Compound 6.

FIG. 30 is a bar graph showing near complete suppression of the KLH-specific IgG response on day 14 in immunized animals administered cyclophosphamide, Compound 5, and Compound 6.

### DEFINITIONS

As used herein, the term “fusion protein” refers to a composite polypeptide made up of two (or more) distinct, heterologous polypeptides. The heterologous polypeptides can either be full-length proteins or fragments of full-length proteins. Fusion proteins herein can be prepared by either synthetic or recombinant techniques known in the art.

As used herein, the term “antibody” refers to an immunoglobulin (Ig) molecule that specifically binds to, or is immunologically reactive with, a particular antigen. The antibody can be, for example, a natural or artificial mono- or polyvalent antibody including, but not limited to, a polyclonal, monoclonal, multi-specific, human, humanized, or chimeric antibody. An antibody may be a genetically engineered or otherwise modified form of an antibody, including but not limited to, heteroconjugate antibodies (e.g., bi-, tri-, and tetra-specific antibodies, diabodies, triabodies, and tetrabodies), and antigen binding fragments of antibodies, including, for example, single domain, Fab′, F(ab′)2, Fab, Fv, rIgG and scFv fragments.

As used herein, the term “affinity” refers to the strength of an interaction between binding moiety and its target. For example, an Fc receptor binding domain interacts through non-covalent forces with an Fc receptor (e.g., FcRn, FcγRI, FcγRII, or FcγRIII). As used herein, the term “high affinity” for an Fc receptor binding domain or fragment thereof (e.g., an Fc domain) refers to an Fc receptor binding domain having a K_{D} of 10⁻⁸ M or less, 10⁻⁹ M or less, 10⁻¹⁰ M or less, 10⁻¹¹ M or less, 10⁻¹² M or less, or 10⁻¹³ M or less for an Fc receptor. As used herein, the term “low affinity” for an Fc receptor binding domain or fragment thereof (e.g., an Fc domain) refers to an Fc receptor binding domain having a K_{D} of 10⁻⁷ M or more, 10⁻⁶ M or more, or 10⁻⁵ M or more for an Fc receptor.

The term “Fc domain,” “Fc receptor binding domain,” or “Fc region of an antigen-binding molecule” as used herein refers to an Fc receptor binding domain that directly binds to an Fc receptor (e.g., FcRn, FcγRI, FcγRII, or FcγRIII), including to a mammalian Fc receptor (e.g., a human Fc receptor). In particular, an Fc domain is an Fc domain of an antibody. Exemplary Fc domains include an Fc domain comprising the second and third constant domain of a human Ig (CH2 and CH3), or the hinge, CH2 and CH3. The Ig may be an IgG (e.g., human IgG1).

The term “Fc receptor” as used herein refers to a protein on the surface of immune cells, such as natural killer cells, macrophages, neutrophils, and mast cells. An Fc receptor can bind to an Fc (Fragment, crystallizable) region of an antibody that is complexed with infected cells or invading pathogens and this binding can stimulate phagocytic or cytotoxic cells to eliminate microbes, or infected cells by antibody-mediated phagocytosis or antibody-dependent cell-mediated cytotoxicity. There are several different types of Fc receptors, which are classified based on the type of antibody that they recognize. Herein, the term “FcRn” refers to the neonatal Fc receptor that binds IgG; other Fc receptors include FcγRI, FcγRII, and FcγRIII. FcRn is similar in structure to MHC class I protein, which, in humans, is encoded by the FCGRT gene. An Fc domain from an antibody includes a receptor binding domain that binds directly to FcRn. Peptides or other molecules can, for example, be fused or attached to, for example, an Fc domain from an antibody or albumin, to provide indirect FcRn binding to the peptide or other molecule. A human FcRn-binding region can be a region that binds to a polypeptide having human FcRn-binding activity.

As used herein, the term “fused” or “joined” refers to the combination or attachment of two or more elements, components, or protein domains, e.g., polypeptides, by means including chemical conjugation, recombinant means, and chemical bonds, e.g., disulfide bonds and amide bonds. For example, two single polypeptides can be joined to form one contiguous protein structure through chemical conjugation, a chemical bond, a peptide linker, or any other means of covalent linkage.

As used herein, the term “linker” refers to a linkage between two elements, e.g., polypeptides or protein domains. A linker can be a covalent bond. A linker can also be a molecule of any length that can be used to couple, for example, a factor H fragment with an Fc receptor binding domain. A linker also refers to a moiety (e.g., a polyethylene glycol (PEG) polymer) or an amino acid sequence (e.g., a 1-200 amino acid, 1-150 amino acid, 1-100, a 5-50, 1-10 or a 1-5 amino acid sequence) occurring between two polypeptides or polypeptide domains to provide space and/or flexibility between the two polypeptides or polypeptide domains. An amino acid linker may be part of the primary sequence of a polypeptide (e.g., joined to the linked polypeptides or polypeptide domains via the polypeptide backbone).

As used herein, the term “host cell” refers to any kind of cellular system that can be engineered to generate the fusion proteins described herein.

As used herein, the term “operatively linked” in the context of a polynucleotide fragment means that the two polynucleotide fragments are joined such that the amino acid sequences encoded by the two polynucleotide fragments remain in-frame.

As used herein, the term “alternative complement pathway” refers to one of three pathways of complement activation (the others being the classical pathway and the lectin pathway).

As used herein, the term “alternative complement pathway dysregulation” refers to any aberration in the ability of the alternative complement pathway to provide host defense against pathogens and clear immune complexes and damaged cells and for immunoregulation. Alternative complement pathway dysregulation can occur in the fluid phase and at the cell surface and can lead to excessive complement activation or insufficient regulation, both causing tissue injury.

As used herein, the term “human properdin” refers to a 469 amino acid soluble glycoprotein found in plasma that has seven thrombospondin repeats type I (TSR) with the N-terminal domain, TSR0, being a truncated domain. Human properdin, a 53 kDa protein, includes a signal peptide (amino acids 1-28), and six, non-identical TSR repeats about 60 amino acids each, as follows: amino acids 80-134 (TSR1), amino acids 139-191 (TSR2), amino acids 196-255 (TSR3), amino acids 260-313 (TSR4), amino acids 318-377 (TSR5), and amino acids 382-462 (TSR6). Properdin is formed by oligomerization of a rod-like monomer into cyclic dimers, trimers, and tetramers. The amino acid sequence of human properdin is found in the GenBank database under the following accession numbers: for human properdin, see, e.g., GenBank Accession Nos. AAA36489, NP_002612, AAH15756, AAP43692, S29126 and CAA40914. Properdin is a positive regulator of the alternative complement activation cascade. Known binding ligands for properdin include C3b, C3bB and C3bBb (Blatt, A. et al., Immunol. Rev., 274:172-90, 2016).

As used herein, “Factor H” refers to a protein component of the alternative complement pathway encoded by the complement Factor H gene (“CFH;” NM000186; GeneID:3075; UniProt ID P08603; Ripoche, J. et al., Biochem. J., 249:593-602, 1988). Factor H is translated as a 1,213 amino acid precursor polypeptide that is processed by removal of an 18 amino acid signal peptide, resulting in the mature Factor H protein (amino acids 19-1231). Factor H consists of 20 short consensus repeat (SCR) domains. Amino acids 1-18 comprise the signal peptide, residues 21-80 comprise SCR1 (EDCNELPPRRNTEILTGSWSDQTYPEGTQAIYKCRPGYRSLGNVIMVCRKGEWVALNPLR KCQK; SEQ ID NO: 1), residues 85-141 comprise SCR 2 (RPCGHPGDTPFGTFTLTGGNVFEYGVKAVYTCNEGYQLLGEINYRECDTDGWTNDIPICEV; SEQ ID NO: 2), residues 146-205 comprise SCR3 (VKCLPVTAPENGKIVSSAMEPDREYHFGQAVRFVCNSGYKIEGDEEMHCSDDGFWSKEKP KCVE; SEQ ID NO: 3), residues 201-262 comprise SCR 4 (ISCKSPDVINGSPISQKIIYKENERFQYKCNMGYEYSERGDAVCTESGWRPLPSCEE; SEQ ID NO: 4), and residues 267-320 comprise SCR 5 (KSCDNPYIPNGDYSPLRIKHRTGDEITYQCRNGFYPATRGNTAKCTSTGWIPAPRCTLK; SEQ ID NO: 5). Factor H regulates alternative complement pathway activation on self-cells by possessing both cofactor activity for the Factor I-mediated C3b cleavage, and decay accelerating activity against the alternative pathway C3 convertase, C3bBb.

As used herein, a “functional fragment” or a “biologically active fragment” refers to a fragment, or portion, of a protein having some or all of the activities of the full-length protein. For example, a functional or biologically active fragment of Factor H, refers to any fragment of a Factor H protein having some or all of the activities of Factor H, e.g., alternative complement pathway regulatory activity of the full-length Factor H protein. Examples include, but are not limited to, Factor H fragments, joined from N-terminus to C-terminus, containing the following SCRs: [1-4] and [1-5].

As used herein, the term “fragment” refers to less than 100% of the amino acid sequence or a full-length reference protein (e.g., 99%, 95%, 90%, 80%, 70%, 60%, 50%, 40%, 30%, 20%, 10%, of the full-length sequence, etc.), but including, e.g., 20, 25, 30, 35, 40, 45, 50, 100, 150, 200, 250, 300, 350, or more amino acids. A fragment can be of sufficient length such that a desirable function of the full-length protein is maintained. For example, the regulation of the alternative complement pathway in fluid phase by fragments of, for example, Factor H, is maintained. Such fragments are “biologically active fragments.”

As used herein, the terms “short complement regulator,” or “SCR,” also known as “short consensus repeat,” or “complement control protein” describe domains found in all regulators of complement activation (RCA) gene clusters that contribute to their ability to regulate complement activation in the blood or on the cell surface. SCRs typically are composed of about 60 amino acids, with four cysteine residues disulfide bonded in a 1-3, 2-4 arrangement and a hydrophobic core built around an almost invariant tryptophan residue. SCRs are found in proteins including, but not limited to, Factor H.

As used herein, the term “disease” refers to an interruption, cessation, or disorder of body functions, systems, or organs. Disease(s) or disorders of interest include those that would benefit from treatment with a fusion protein or method described herein. Non-limiting examples of diseases or disorders to be treated herein resulting from the dysregulation of the alternative complement pathway activation include, for example, paroxysmal nocturnal hemoglobinuria (PNH), atypical hemolytic uremic syndrome (aHUS), IgA nephrology, lupus nephritis, C3 glomerulopathy (C3G), dermatomyositis, systemic sclerosis, demyelinating polyneuropathy, pemphigus, membranous nephropathy, focal segmental glomerular sclerosis (FSGS), bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, dense deposit disease (DDD), age related macular degeneration (AMD), systemic lupus erythematosus (SLE), rheumatoid arthritis (RA), multiple sclerosis (MS), traumatic brain injury (TBI), ischemia reperfusion injury, preeclampsia, or thrombic thrombocytopenia purpura (TTP).

As used herein, the terms “treatment,” “treating,” or “treat” refer to therapeutic treatment, in which the object is to inhibit or lessen an undesired physiological change or disorder or to promote a beneficial phenotype in a subject. For example, “treatment,” “treating” or “treat” refer to clinical intervention in an attempt to alter the natural course of an individual's affliction, disease, or disorder. The terms include, for example, prophylaxis before or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration, or palliation of the disease state, and improved prognosis. In some embodiments, fusion proteins are used to control the cellular and clinical manifestations of PNH, aHUS, IgA nephrology, lupus nephritis, C3G, dermatomyositis, systemic sclerosis, demyelinating polyneuropathy, pemphigus, membranous nephropathy, FSGS, bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, DDD, AMD, SLE, RA, MS, TBI, ischemia reperfusion injury, preeclampsia, or TTP

As used herein, “administering” and “administration” refers refer to any method of providing a pharmaceutical preparation to a subject. Fusion proteins may be administered by any method known to those skilled in the art. Suitable methods for administering the fusion protein may be, for example, orally, by injection (e.g., intravenously, intraperitoneally, intramuscularly, intravitreally, and subcutaneously), drop infusion preparations, and the like. Fusion proteins prepared as described herein may be administered in various forms, depending on the disorder to be treated and the age, condition, and body weight of the subject, as is known in the art. A preparation can be administered prophylactically; that is, administered to decrease the likelihood of developing a disease or condition.

As used herein, the term “effective amount” refers to an amount that is sufficient to achieve the desired result or to have an effect on an undesired condition. For example, an “effective amount” refers to an amount that is sufficient to achieve the desired therapeutic result. The specific therapeutically effective dose for any particular subject depends upon a variety of factors including the disorder being treated and the severity of the disorder; the specific composition employed; the age, body weight, general health, sex, and diet of the subject; the time of administration; the route of administration; the rate of excretion of the specific compound employed; the duration of the treatment; drugs used in combination or coincidental with the specific compound employed, and like factors known in the art. Dosage can vary, and can be administered in one or more dose administrations daily, weekly, monthly, or yearly, for one or several days.

As used herein, the term “patient in need thereof” or “subject in need thereof,” refers to the identification of a subject based on need for treatment of a disease or disorder. A subject can be identified, for example, as having a need for treatment of a disease or disorder (e.g., PNH, aHUS, IgA nephrology, lupus nephritis, C3G, dermatomyositis, systemic sclerosis, demyelinating polyneuropathy, pemphigus, membranous nephropathy, FSGS, bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, DDD, AMD, SLE, RA, MS, TBI, ischemia reperfusion injury, preeclampsia, or TTP, based upon an earlier diagnosis by a person of skill in the art (e.g., a physician).

### DETAILED DESCRIPTION

Described herein are alternative complement pathway-specific C3 and C5 convertase inhibitors that regulate alternative complement pathway activity. Diseases mediated by complement dysregulation are often a result of complement overactivity both in the fluid phase and at the cell surface. Described herein are compositions and methods for treating diseases mediated by complement dysregulation. Examples of disorders mediated by alternative complement pathway dysregulation include, for example paroxysmal nocturnal hemoglobinuria (PNH), atypical hemolytic uremic syndrome (aHUS), IgA nephrology, lupus nephritis, C3 glomerulopathy (C3G), dermatomyositis, systemic sclerosis, demyelinating polyneuropathy, pemphigus, membranous nephropathy, focal segmental glomerular sclerosis (FSGS), bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, dense deposit disease (DDD), age related macular degeneration (AMD), systemic lupus erythematosus (SLE), rheumatoid arthritis (RA), multiple sclerosis (MS), traumatic brain injury (TBI), ischemia reperfusion injury, preeclampsia, or thrombic thrombocytopenia purpura (TTP)

The compositions and methods described herein feature fusion proteins that include complement factor H (FH) or a functional fragment of FH fused to an Fc receptor binding domain (e.g., a monoclonal antibody, or fragment thereof (e.g., an Fc domain)). Exemplary fusion proteins for use in the methods of treatment described herein include, but are not limited to, Compound 1, Compound 2, Compound 3, Compound 4, Compound 5, Compound 7, Compound 8, Compound 9, Compound 10, and Compound 11 (e.g., a fusion protein having the amino acid sequence of any one of SEQ ID NOs: 98-107; or a fusion protein encoded by the nucleic acid sequence of any one of SEQ ID NOS: 110-118), or variants of these compounds described herein.

The fusion protein regulates alternative complement pathway activity, by attenuating C3 and C5 convertase activity. Moreover, the Fc receptor binding domain increases the serum half-life of the fusion protein. The overall design targets the alternative complement pathway and leaves activation (protection) via classical and lectin pathways intact.

### Factor H Fusion Proteins

As described herein, fusion proteins that include factor H or a functional fragment of FH and an Fc receptor binding domain (e.g., an IgG or a functional fragment thereof, e.g., an Fc domain) can be used as therapeutic agents to treat diseases mediated by alternative complement pathway dysregulation. In humans, several regulatory proteins are encoded by a cluster of genes located on the long arm of chromosome 1. This region is called the regulator of complement activation (RCA) gene cluster. Although the proteins within the RCA family vary in size, they share significant primary amino acid structure similarities. The best studied members of the RCA family are factor H, factor H-like protein 1 (FHL-1), complement receptor 1 (CR1), decay accelerating factor (DAF), membrane cofactor protein (MCP), and C4b-binding protein (C4BP). The members are organized in tandem structural units termed short consensus repeats (SCRs), which are present in multiple copies in the protein. Each SCR consists of 60-70 highly conserved amino acids, including four cysteines.

In certain embodiments, the portion of the fusion protein suitable for inhibiting activity of the alternative complement pathway includes a fragment of factor H. In certain embodiments, the biologically active fragment of factor H includes the first four N-terminal SCR domains of factor H (e.g., SCRs 1, 2, 3, and 4, (SEQ ID NO: 91)). In certain embodiments, the biologically active fragment of factor H includes the first five N-terminal SCR domains of factor H (e.g., SCRs 1, 2, 3, 4, and 5 (SEQ ID NO: 92)). In some embodiments, the fragment of factor H portion of the fusion protein composition is a functional fragment of wild-type factor H.

Exemplary fusion proteins include, but are not limited to: (i) one in which factor H is fused to an Fc receptor binding domain, or fragment thereof (e.g., an Fc domain) or (ii) one in which a functional fragment of factor H is fused to an Fc receptor binding domain, or fragment thereof (e.g., an Fc domain), wherein the fragment of factor H includes the first four N-terminal SCR domains of factor H; or (iii) one in which a functional fragment of factor H is fused to an Fc receptor binding domain, or fragment thereof (e.g., an Fc domain), wherein the fragment of factor H includes the first five N-terminal SCR domains of factor H, or (iv) variants in which a functional fragment of factor H (e.g., a fragment of factor H that includes the first four or five N-terminal SCR domains of factor H) is fused to an Fc receptor binding domain, (e.g., an antibody or fragment thereof (e.g., an Fc domain)), or (v) one in which a functional fragment of factor H (e.g., a fragment of factor H that includes the first four or five N-terminal SCR domains of factor H) is fused to an Fc receptor binding domain, wherein an amino acid of the fragment of factor H includes an additional sequence, such as a leader or secretory sequence or a sequence that is employed for purification of the composition, or (vi) one in which a functional fragment of factor H (e.g., a fragment of factor H that includes the first four or five N-terminal SCR domains of factor H) is fused to an Fc receptor binding domain, wherein an amino acid of the fragment of factor H is fused with a larger polypeptide, e.g., human albumin, an antibody, or an Fc, for increased duration of effect. Such fusion proteins are deemed to be within the scope of those skilled in the art from the teachings herein.

The fragment of factor H may be prepared by a number of synthetic methods of peptide synthesis by fragment condensation of one or more amino acid residues, according to conventional peptide synthesis methods known in the art (Amblard et al., Mol. Biotechnol., 33:239-54, 2006).

Alternatively, a fragment of factor H may be produced by expression in a suitable prokaryotic or eukaryotic system. In some embodiments, a DNA construct may be inserted into a plasmid vector adapted for expression in a suitable host cell (such as E. coli, a mammalian cell (e.g., CHO, or a yeast cell (such as S. cerevisiae)), into a baculovirus vector for expression in an insect cell, or a viral vector for expression in a mammalian cell (e.g., CHO). Such vectors include the regulatory elements necessary and sufficient for expression of the DNA in the host cell. The fragment of factor H produced by gene expression in a recombinant prokaryotic or eukaryotic system may be purified according to methods known in the art (Structural Genomics Consortium, Nat. Methods, 5:135-46, 2008).

### Ig Proteins and Fc Receptor Binding Domains

Factor H fusion proteins, as described herein, include factor H or a functional fragment of factor H fused to an Fc receptor binding domain. In some embodiments, the Fc receptor binding domain is an antibody, or a functional fragment thereof. The antibody that can be incorporated into a fusion protein as the Fc receptor binding domain may be an IgA, IgD, IgE, IgG, or IgM antibody.

The fusion proteins described herein may utilize a wide variety of antibodies or antibody fragments containing Fc receptor binding domain. In some instances, the Fc receptor binding domain includes a complete monoclonal antibody (e.g., an IgG). In some embodiments, the Fc receptor binding domain includes only the Fc domain of an antibody. In some embodiments, the full-length antibody (e.g., an IgG molecule) can comprise, for example, a constant region, or a portion thereof, from any type of antibody isotype, including, for example, IgG (including IgG1, IgG2, IgG3, and IgG4), or a hybrid constant region, or a portion thereof (e.g., a chimera), such as a IgG2/G4 hybrid constant region (Burton, D. & Woof, J., Adv. Immun., 51:1-18, 1992; Canfield S. & Morrison, S., J. Exp. Med., 173:1483-91, 1991; Mueller J. et al., Mol. Immunol., 34:441-52, 1997). Exemplary Fc domains include an Fc region comprising the second and third constant domain of a human immunoglobulin (CH2 and CH3), or the hinge, CH2, and CH3. The Fc domain may be that from an IgG (e.g., human IgG1). For example, the Fc domain may be an IgG 2/4 Fc domain having the sequence VECPPCPAPPVAGPSVFLFPPKPKDTLM ISRTPEVTCVVVDVSQEDPEVQFNWYVDGVEVHNAKTKPREEQFNSTYRVVSVLTVLHQDWLNGKEYK CKVSNKGLPSSIEKTISKAKGQPREPQVYTLPPSQEEMTKNQVSLTCLVKGFYPSDIAVEWESNGQPENN YKTTPPVLDSDGSFFLYSRLTVDKSRWQEGNVFSCSVMHEALHNHYTQKSLSLSLGK (SEQ ID NO: 87) or ERKCCVECPPCPAPPVAGPSVFLFPPKPKDTLMISRTPEVTCVVVDVSQEDPEVQFNWYVDGVEVH NAKTKPREEQFNSTYRVVSVLTVLHQDWLNGKEYKCKVSNKGLPSSIEKTISKAKGQPREPQVYTLPPSQ EEMTKNQVSLTCLVKGFYPSDIAVEWESNGQPENNYKTTPPVLDSDGSFFLYSRLTVDKSRWQEGNVFS CSVMHEALHNHYTQKSLSLSLGK (SEQ ID NO: 88). Additional exemplary Fc domains include a proline-stabilized hinge, CH2, and CH3 of IgG4 having the sequence ESKYGPPCPPCPAPEFLGGPS VFLFPPKPKDTLMISRTPEVTCVVVDVSQEDPEVQFNWYVDGVEVHNAKTKPREEQFNSTYRVVSVLTV LHQDWLNGKEYKCKVSNKGLPSSIEKTISKAKGQPREPQVYTLPPSQEEMTKNQVSLTCLVKGFYPSDIA VEWESNGQPENNYKTTPPVLDSDGSFFLYSRLTVDKSRWQEGNVFSCSVMHEALHNHYTQKSLSLSLG K (SEQ ID NO: 89). The Fc domain may be that from an IgG (e.g., human IgG1, e.g., of the hinge, CH2, and CH3 regions of IgG1 having the sequence of AEPKSDKTHTCPPCPAPELLGGPSVFLFPPKPKDTLM ISRTPEVTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGFYPSDIAVEWESNGQPENN YKTTPPVLDSDGSFFLYSKLTVDKSRWQQGNVFSCSVMHEALHNHYTQKSLSLSPGK (SEQ ID NO: 90)). In some embodiments, the factor H fusion protein including an Fc receptor binding domain has an increased half-life relative to the fusion protein lacking the Fc receptor binding domain.

### Linkers for the Fusion Proteins

A linker is used to create a linkage or connection between, for example, polypeptides, or protein domains. For example, factor H or a functional fragment of factor H may be linked directly to an Fc receptor binding domain (e.g., an IgG, or a functional fragment thereof, e.g., an Fc domain) by one or more suitable linkers. A linker can be a simple covalent bond, e.g., a peptide bond, a synthetic polymer, e.g., a PEG polymer, or any kind of bond created from a chemical reaction, e.g., chemical conjugation. The peptide linker can be, for example, a linker of one or more amino acid residues inserted or included at the transition between the two domains (e.g., a fragment of the FH domain and an Fc receptor binding domain). The identity and sequence of amino acid residues in the linker may vary depending on the desired secondary structure. For example, glycine, serine, and alanine are useful for linkers given their flexibility. Any amino acid residue can be considered as a linker in combination with one or more other amino acid residues, which may be the same as or different from the first amino acid residue, to construct larger peptide linkers as necessary depending on the desired length and/or properties.

A variety of linkers can be used to fuse two or more protein domains together (e.g., a fragment of factor H and an Fc receptor binding domain). Linkers may be flexible, rigid, or cleavable. Linkers may be structured or unstructured. The residues for the linker may be selected from naturally occurring amino acids, non-naturally occurring amino acids, and modified amino acids. The linker may include at least 1 or more, 2 or more, 5 or more, 10 or more, 15 or more, or 20 or more amino acid residues. Peptide linkers can include, but are not limited to, glycine linkers, glycine-rich linkers, serine-glycine linkers, and the like. A glycine-rich linker includes at least about 50% glycine.

In some embodiments, the linker(s) used confer one or more other favorable properties or functionality to the polypeptide(s) described herein, and/or provide one or more sites for the formation of derivatives and/or for the attachment of functional groups. For example, linkers containing one or more charged amino acid residues can provide improved hydrophilic properties, whereas linkers that form or contain small epitopes or tags can be used for the purposes of detection, identification, and/or purification. A skilled artisan can determine the optimal linkers for use in a specific polypeptide.

When two or more linkers are used for a polypeptide, the linkers may be the same or different.

Linkers can contain motifs, e.g., multiple or repeating motifs. In one embodiment, the linker has the amino acid sequence GS, or repeats thereof (Huston et al., Methods Enzymol., 203:46-88, 1991). In another embodiment, the linker includes the amino acid sequence EK, or repeats thereof (Whitlow et al., Protein Eng., 6:989-95, 1993). In another embodiment, the linker includes the amino acid sequence GGS, or repeats thereof.

In another embodiment, the linker includes the amino acid sequence GGGGS (SEQ ID NO: 6) or repeats thereof. In certain embodiments, the linker contains more than one repeat of GGS or GGGGS (U.S. Pat. No. 6,541,219, the entire contents of which are herein incorporated by reference). In one embodiment, the peptide linker may be rich in small or polar amino acids, such as G and S, but can contain additional amino acids, such as T and A, to maintain flexibility, as well as polar amino acids, such as K and E, to improve solubility.

In one embodiment, the linker is a cleavable linker, such as an enzymatically cleavable linker. Inclusion of a cleavable linker can aid in detection of the fusion protein. An enzymatically cleavable linker can be cleavable, for example, by trypsin, Human Rhinovirus 3C Protease (3C), enterokinase (Ekt), Factor Xa (FXa), Tobacco Etch Virus protease (TEV), or thrombin (Thr). Cleavage sequences for each of these enzymes are well known in the art. For example, trypsin cleaves peptides on the C-terminal side of lysine and arginine amino acid residues. If a proline residue is on the carboxyl side of the cleavage site, the cleavage will not occur. If an acidic residue is on either side of the cleavage site, the rate of hydrolysis has been shown to be slower. The following linkers are examples of linkers that would be excised using trypsin: K(G₄A)₂G₃AG₄SK (SEQ ID NO:110), R(G₄A)₂G₃AG₄SR (SEQ ID NO:111), K(G₄A)₂G₃AG₄SR (SEQ ID NO:112), R(G₄A)₂G₃AG₄SK (SEQ ID NO:113), K(G₄A)₂G₄SK (SEQ ID NO:114), K(G₄A)₂G₄SR (SEQ ID NO:115), R(G₄A)₂G₄SK (SEQ ID NO:116, and R(G₄A)₂G₄SR (SEQ ID NO:116).

A particular example of a protease cleavage site that can be included in an enzymatically cleavable linker is a tobacco etch virus (TEV) protease cleavage site, e.g., ENLYTQS (SEQ ID NO: 93), where the protease cleaves between the glutamine and the serine. Another example of a protease cleavage site that can be included in an enzymatically cleavable linker is an enterokinase cleavage site, e.g., DDDDK (SEQ ID NO: 94), where cleavage occurs after the lysine residue. Another example of a protease cleavage site that can be included in an enzymatically cleavable linker is a thrombin cleavage site, e.g., LVPR (SEQ ID NO: 95). For Human Rhinovirus 3C Protease, the cleavage site is LEVLFQGP (SEQ ID NO: 96) where cleavage occurs between the glutamine and glycine residues. The preferred cleavage site for Factor Xa protease is IEDGR (SEQ ID NO: 97), where cleavage occurs between the glutamic acid and aspartic acid residues.

The inclusion of the cleavable linker is useful in that it has a sequence of amino acids that is unique from other peptides in the human proteome that are generated with the above mentioned enzymes. As such this excised linker may serve as a unique identifying peptide of the fusion protein when administered as a pharmaceutical preparation to humans. In this way the cleavable linker may be detected and quantitated by mass spectrometry and be used to monitor the pharmacokinetics of the fusion protein.

In another embodiment, the linker is a polymeric or oligomeric glycine linker, and can include a lysine at the N-terminus, the C-terminus, or both the N- and the C-termini.

Exemplary constructs include Fc-L1-FH and FH-L1-Fc, where L1 is a cleavable linker (e.g., enzymatically cleavable linker), as well as Fc-L1-E1-FH and FH-L1-E1-Fc, where L1 is a polymeric or oligomeric glycine linker optionally containing lysine at the N-terminus or C-terminus or both the N- and the C-termini, and E1 is an enzymatically cleavable linker. In each of these constructs, the “FH” component can be, for example, SCR1-4, SCR1-5, or factor H.

Exemplary linkers include, but are not limited to: (G₄A)₂G₃AG₄S (SEQ ID NO: 108), G₄AG₃AG₄S (SEQ ID NO: 109), (G₄A)₂G₄S (SEQ ID NO: 7), G₄SDAA (SEQ ID NO: 8), G₄S (SEQ ID NO: 9), (G₄S)₂ (SEQ ID NO: 10), (G₄S)₃ (SEQ ID NO: 11), (G₄S)₄ (SEQ ID NO: 12), (G₄S)₅ (SEQ ID NO: 13), (G₄S)₆ (SEQ ID NO: 14), EAAAK (SEQ ID NO: 85) (EAAAK)₃ (SEQ ID NO: 15), PAPAP (SEQ ID NO: 16), G₄SPAPAP (SEQ ID NO: 17), PAPAPG₄S (SEQ ID NO: 18), GSTSGKSSEGKG (SEQ ID NO: 19), (GGGDS)₂ (SEQ ID NO: 20), (GGGES)₂ (SEQ ID NO: 21), GGGDSGGGGS (SEQ ID NO: 22), GGGASGGGGS (SEQ ID NO: 23), GGGESGGGGS (SEQ ID NO: 24), ASTKGP (SEQ ID NO: 25), ASTKGPSVFPLAP (SEQ ID NO: 26), G₃P (SEQ ID NO: 27), G₇P (SEQ ID NO: 28), PAPNLLGGP (SEQ ID NO: 29), G₆ (SEQ ID NO: 30), G₁₂ (SEQ ID NO: 31), APELPGGP (SEQ ID NO: 32), SEPQPQPG (SEQ ID NO: 33), (G₃S₂)₃ (SEQ ID NO: 34), GGGGGGGGGGGGS (SEQ ID NO: 35), GGGGSGGGGGGGGGS (SEQ ID NO: 36), (GGSSS)₃ (SEQ ID NO: 37), (GS₄)₃ (SEQ ID NO: 38), G₄A(G₄S)₂ (SEQ ID NO: 39), G₄SG₄AG₄ (SEQ ID NO:40), G₄SG₄AG₄S (SEQ ID NO: 118), G₃AS(G₄S)₂ (SEQ ID NO: 41), G₄SG₃ASG₄S (SEQ ID NO: 42), G₄SAG₃SG₄S (SEQ ID NO: 43), (G₄S)₂AG₃S (SEQ ID NO: 44), G₄SAG₃SAG₃S (SEQ ID NO: 45), G₄D(G₄S)₂ (SEQ ID NO: 46), G₄SG₄DG₄S (SEQ ID NO: 47), (G₄D)₂G₄S (SEQ ID NO: 48), G₄E(G₄S)₂ (SEQ ID NO: 49), G₄SG₄EG₄S (SEQ ID NO: 50), and (G₄E)₂G₄S (SEQ ID NO: 51), (GGGGS)ₙ, wherein n can be any number, KESGSVSSEQLAQFRSLD (SEQ ID NO: 52), and EGKSSGSGSESKST (SEQ ID NO: 53), (Gly)₈ (SEQ ID NO: 54), GSAGSAAGSGEF (SEQ ID NO: 55), and (Gly)₆ (SEQ ID NO: 56). Exemplary rigid linkers include but are not limited to A(EAAAK)A (SEQ ID NO: 86), A(EAAAK)ₙA (SEQ ID NO: 57), wherein n can be any number, or (XP)ₙ wherein n can be any number, with X designating any amino acid. Exemplary in vivo cleavable linkers include, for example, LEAGCKNFFPRSFTSCGSLE (SEQ ID NO: 58), GSST (SEQ ID NO: 59), and CRRRRRREAEAC (SEQ ID NO: 60). In some embodiments, a linker can contain 2 to 12 amino acids including motifs of GS, e.g., GS, GSGS (SEQ ID NO: 61), GSGSGS (SEQ ID NO: 62), GSGSGSGS (SEQ ID NO: 63), GSGSGSGSGS (SEQ ID NO: 64), or GSGSGSGSGSGS (SEQ ID NO: 65). In certain other embodiments, a linker can contain 3 to 12 amino acids including motifs of GGS, e.g., GGS, GGSGGS (SEQ ID NO: 66), GGSGGSGGS (SEQ ID NO: 67), and GGSGGSGGSGGS (SEQ ID NO: 68). In yet other embodiments, a linker can contain 4 to 12 amino acids including motifs of GGSG, e.g., GGSG (SEQ ID NO: 69), GGSGGGSG (SEQ ID NO: 70), or GGSGGGSGGGSG (SEQ ID NO: 71). In other embodiments, a linker can contain motifs of GGGGS (SEQ ID NO: 6), e.g., GGGGSGGGGGGGGS (SEQ ID NO: 72). In other embodiments, a linker can also contain amino acids other than glycine and serine, e.g., GENLYFQSGG (SEQ ID NO: 73), SACYCELS (SEQ ID NO: 74), RSIAT (SEQ ID NO: 75), RPACKIPNDLKQKVMNH (SEQ ID NO: 76), GGSAGGSGSGSSGGSSGASGTGTAGGTGSGSGTGSG (SEQ ID NO: 77), AAANSSIDLISVPVDSR (SEQ ID NO: 78), GGSGGGSEGGGSEGGGSEGGGSEGGGSEGGGSGGGS (SEQ ID NO: 79), GGGGAGGGGAGGGGAGGGGS (SEQ ID NO: 80), DAAGGGGSGGGGSGGGGSGGGGSGGGGS (SEQ ID NO: 81), GGGGAGGGGAGGGGA (SEQ ID NO: 82), GGGGAGGGGAGGGAGGGGS (SEQ ID NO: 83), or GGSSRSSSSGGGGAGGGG (SEQ ID NO: 84).

In a certain embodiment, the C-terminus of factor H or a functional fragment of factor H (e.g., SCR1-4 or SCR1-5) may be linked directly to the N-terminus of an Fc receptor binding domain (e.g., an IgG, or a functional fragment thereof, e.g., an Fc domain). In a certain embodiment, the N-terminus of factor H or a functional fragment of factor H (e.g., SCR1-4 or SCR1-5) may be linked directly to the C-terminus of an Fc receptor binding domain (e.g., an IgG, or a functional fragment thereof, e.g., an Fc domain). In a certain embodiment, the C-terminus of factor H or a functional fragment of factor H (e.g., SCR1-4 or SCR1-5) may be linked directly to the N-terminus of an Fc receptor binding domain (e.g., an Fc domain). In a certain embodiment, the N-terminus of factor H or a functional fragment of factor H (e.g., SCR1-4 or SCR1-5) may be linked directly to the C-terminus of an Fc receptor binding domain (e.g., an Fc domain).

### Production of Fusion Proteins

Described herein are methods for producing a fusion protein using nucleic acid molecules encoding the fusion proteins. The nucleic acid molecule can be operably linked to a suitable control sequence to form an expression unit encoding the protein. The expression unit is used to transform a suitable host cell, and the transformed host cell is cultured under conditions that allow the production of the recombinant protein. Optionally, the recombinant protein is isolated from the medium or from the cells; recovery and purification of the protein may not be necessary in some instances where some impurities may be tolerated.

The fusion proteins of the present disclosure may include naturally-occurring or a non-naturally-occurring components; preferably at least one component is non-naturally occurring, e.g., with respect to its structure (e.g., sequence) and/or its association (e.g., how it is linked to other components). As used herein, the term “non-naturally occurring” refers to any molecule, e.g., fusion protein, produced with the aid of human manipulation, including, without limitation, molecules produced by genetic engineering using random mutagenesis or rational design and molecules produced by chemical synthesis. Non-limiting examples of non-naturally occurring molecules include, e.g., conservatively substituted variants, non-conservatively substituted variants, and active hybrids (e.g., chimeras) or fragments. Non-natural molecules further include natural molecules that have been modified, e.g., post-translationally, e.g., via addition of chemical moieties, tags, ligands. Preferably, non-natural molecules include the fusion proteins of the present disclosure.

The fusion protein can be expressed from a single polynucleotide that encodes the entire fusion protein or as multiple (e.g., two or more) polynucleotides that may be expressed by suitable expression systems or may be co-expressed. Polypeptides encoded by polynucleotides that are co-expressed may associate through, e.g., disulfide bonds or other means to form a functional fusion protein. For example, the light chain portion of monoclonal antibody may be encoded by a separate polynucleotide from the heavy chain portion of a monoclonal antibody. When co-expressed in a host cell, the heavy chain polypeptides associate with the light chain polypeptides to form the monoclonal antibody.

It is envisioned that any and all polynucleotide molecules that can encode the fusions disclosed in the present specification can be useful, including, without limitation naturally-occurring and non-naturally-occurring DNA molecules and naturally-occurring and non-naturally-occurring RNA molecules. Non-limiting examples of naturally-occurring and non-naturally-occurring DNA molecules include single-stranded DNA molecules, double-stranded DNA molecules, genomic DNA molecules, cDNA molecules, vector constructs, such as, e.g., plasmid constructs, phagemid constructs, bacteriophage constructs, retroviral constructs and artificial chromosome constructs. Non-limiting examples of naturally-occurring and non-naturally-occurring RNA molecules include single-stranded RNA, double stranded RNA and mRNA. The present disclosure also provides synthetic nucleic acids, e.g., non-natural nucleic acids, comprising nucleotide sequence encoding one or more of the aforementioned fusion proteins. Included herein are nucleic acids encoding the fusion proteins, including the complementary strand thereto, or the RNA equivalent thereof, or a complementary RNA equivalent thereof.

Typically, a nucleic acid encoding the desired fusion protein is generated using molecular cloning methods, and is generally placed within a vector, such as a plasmid constructs, phagemid constructs, bacteriophage constructs, retroviral constructs and artificial chromosome constructs. Non-limiting examples of naturally-occurring and non-naturally-occurring RNA molecules include single-stranded RNA, double stranded RNA and mRNA.

The vector is used to transform the nucleic acid into a host cell appropriate for the expression of the fusion polypeptide. Methods for creating such vectors and expression techniques are known in the art (Sambrook, J. et al., Molecular cloning: A laboratory manual. Cold Spring Harbor, N.Y: Cold Spring Harbor Laboratory). Many cell types can be used as appropriate host cells, although mammalian cells are preferable because they are able to confer appropriate post-translational modifications. Host cells can include, e.g., a Human Embryonic Kidney (HEK) (e.g., HEK 293) cell, Chinese Hamster Ovary (CHO) cell, L cell, NSO cell, C127 cell, 3T3 cell, BHK cell, COS-7 cell, or any other suitable host cell known in the art.

In addition, prokaryotic cells including, without limitation, strains of aerobic, microaerophilic, capnophilic, facultative, anaerobic, gram-negative and gram-positive bacterial cells such as those derived from, e.g., Escherichia coli, Bacillus subtilis, Bacillus licheniformis, Bacteroides fragilis, Clostridia perfringens, Clostridia difficile, Caulobacter crescentus, Lactococcus lactis, Methylobacterium extorquens, Neisseria meningirulls, Neisseria meningitidis, Pseudomonas fluorescens and Salmonella typhimurium; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; insect cells and cell lines derived from insects, such as, e.g., those derived from Spodoptera frugiperda, Trichoplusia ni, Drosophila melanogaster and Manduca Sexta; and mammalian cells and cell-lines derived from mammalian cells, such as, e.g., those derived from mouse, rat, hamster, porcine, bovine, equine, primate and human may be used. Cell lines may be obtained from the American Type Culture Collection (2004); European Collection of Cell Cultures (2204); and the German Collection of Microorganisms and Cell Cultures (2004).

Included herein are codon-optimized sequences of the aforementioned nucleic acid sequences and vectors. Codon optimization for expression in a host cell, e.g., bacteria such as E. coli or insect Hi5 cells, may be performed using Codon Optimization Tool (CODONOPT), available freely from Integrated DNA Technologies, Inc., Coralville, Iowa, USA.

In one embodiment, a nucleic acid or polynucleotide encoding the fusion protein is provided. In one embodiment, a vector including a nucleic acid or polynucleotide encoding the fusion protein is provided. In one embodiment, a host cell including one or more polynucleotides encoding the fusion protein is provided. In certain embodiments a host cell including one or more fusion expression vectors is provided. The fusion proteins can be produced by expression of a nucleotide sequence in any suitable expression system known in the art. Any expression system may be used, including yeast, bacterial, animal, plant, eukaryotic, and prokaryotic systems. In some embodiments, yeast systems that have been modified to reduce native yeast glycosylation, hyper-glycosylation or proteolytic activity may be used. Furthermore, any in vivo expression systems designed for high level expression of recombinant proteins within organisms known in the art can be used for producing the fusion proteins specified herein. In some embodiments, the factor H fusion protein, as described herein, is produced by culturing one or more host cells including one or more nucleic acid molecules capable of expressing the fusion protein under conditions suitable for expression of the fusion protein. In some embodiments, the factor H fusion protein is obtained from the cell culture or culture medium.

The fusion protein can also be produced using chemical methods to synthesize the desired amino acid sequence, in whole or in part. For example, polypeptides can be synthesized by solid phase techniques, cleaved from the resin, and purified by preparative high performance liquid chromatography (Creighton (1983) Proteins: Structures And Molecular Principles, WH Freeman and Co, New York, NY). The composition of the synthetic polypeptides can be confirmed by amino acid analysis or sequencing. Additionally, the amino acid sequence of a fusion protein or any part thereof, can be altered during direct synthesis and/or combined using chemical methods with a sequence from other subunits, or any part thereof, to produce a variant polypeptide. Additional residues may be included at the N- or C-terminus of the protein-coding sequence to facilitate purification (e.g., a histidine tag or a glutathione S-transferase (GST) tag).

### Isolation/Purification of Fusion Proteins

Secreted, biologically active fusion proteins described herein may be purified by techniques such as high performance liquid chromatography, ion exchange chromatography, gel electrophoresis, affinity chromatography, e.g., protein-A affinity chromatography, size exclusion chromatography, and the like. The conditions used to purify a particular protein depend, in part, on factors such as net charge, hydrophobicity, hydrophilicity etc., as would be apparent to a skilled artisan.

Exemplified Compounds are described in Tables 1-3 below.

TABLE 1
FH-Fc fusion proteins described in the Examples

| | | Second | Compound |
|---|---|---|---|
| First Moiety | Linker | Moiety | Name |
| mIgG1 | | FH 1-4 | Compound 1 |
| | | | (SEQ ID NO: 98 |
| IgG2-G4-Fc | (G₄A)₂G₄S | FH 1-5 | Compound 5 |
| | (SEQ ID NO: 7) | | (SEQ ID NO: 99) |
| hIgG1-Fc | (G₄A)₂G₄S | FH 1-5 | Compound 2 |
| | (SEQ ID NO: 7) | | (SEQ ID NO: 100) |
| hIgG1-Fc | | FH 1-5 | Compound 3 |
| | | | (SEQ ID NO: 101) |
| First Moiety | Linker | Second | Compound |
| | | Moiety | Name |
| FH 1-5 | (G₄A)₂G₄S | IgG1 Fc | Compound 4 |
| | (SEQ ID NO: 7) | | (SEQ ID NO: 102) |
| Peptibody-like hIgG2/G4 | (G₄A)₂G₃AG₄S | FH 1-4 | Compound 7 |
| | (SEQ ID NO: 108) | | (SEQ ID NO: 103) |
| Peptibody-like hIgG2/G4 | (G₄A)₂G₃AG₄S | K-FH 1-4 | Compound 8 |
| | (SEQ ID NO: 108) | | (SEQ ID NO: 104) |
| Peptibody-like hIgG2/G4 | (G₄A)₂G₃AG₄S | R-FH 1-4 | Compound 9 |
| | (SEQ ID NO: 108) | | (SEQ ID NO: 105) |
| Peptibody-like hIgG2/G4 | G₄AG₃AG₄S | K-FH 1-4 | Compound 10 |
| | (SEQ ID NO: 109) | | (SEQ ID NO: 106) |
| Peptibody-like hIgG2/G4 | G₄AG₃AG₄S | R-FH 1-4 | Compound 11 |
| | (SEQ ID NO: 109) | | (SEQ ID NO: 107) |

TABLE 2
Amino acid sequences for FH-Fc fusion
proteins described in the Examples

| | SEQ | |
|---|---|---|
| | ID | |
| Compound | NO. | Amino Acid Sequence |
| Linker | 7 | GGGGAGGGAGGGGS |
| used in | | |
| Compound 10 | | |
| and | | |
| Compound 11 | | |
| Compound 1 | 98 | DKKIVPRDCG CKPCICTVPE VSSVFIFPPK |
| | | PKDVLTITLT PKVTCVVVDI SKDDPEVQFS |
| | | WFVDDVEVHT AQTKPREEQI NSTFRSVSEL |
| | | PIMHQDWLNG KEFKCRVNSA AFPAPIEKTI |
| | | SKTKGRPKAP QVYTIPPPKE QMAKDKVSLT |
| | | CMITNFFPED ITVEWQWNGQ PAENYKNTQP |
| | | IMDTDGSYFV YSKLNVQKSN WEAGNTFTCS |
| | | VLHEGLHNHH TEKSLSHSPG KGGGGAGGGG |
| | | AGGGAGGGGS EDCKGPPPRE NSEILSGSWS |
| | | EQLYPEGTQA TYKCRPGYRT LGTIVKVCKN |
| | | GKWVASNPSR ICRKKPCGHP GDTPFGSFRL |
| | | AVGSQFEFGA KVVYTCDDGY QLLGEIDYRE |
| | | CGADGWINDI PLCEVVKCLP VTELENGRIV |
| | | SGAAETDQEY YFGQVVRFEC NSGFKIEGHK |
| | | EIHCSENGLW SNEKPRCVEI LCTPPRVENG |
| | | DGINVKPVYK ENERYHYKCK HGYVPKERGD |
| | | AVCTGSGWSS QPFCEEKR |
| Compound 5 | 99 | VECPPCPAPPVAGPSVFLFPPKPKDTLMIS |
| | | RTPEVTCVVVDVSQEDPEVQFNWYVDGVEV |
| | | HNAKTKPREEQFNSTYRVVSVLTVLHQDWL |
| | | NGKEYKCKVSNKGLPSSIEKTISKAKGQPR |
| | | EPQVYTLPPSQEEMTKNQVSLTCLVKGFYP |
| | | SDIAVEWESNGQPENNYKTTPPVLDSDGSF |
| | | FLYSRLTVDKSRWQEGNVFSCSVMHEALHN |
| | | HYTQKSLSLSLGKGGGGAGGGGAGGGGSED |
| | | CNELPPRRNTEILTGSWSDQTYPEGTQAIY |
| | | KCRPGYRSLGNVIMVCRKGEWVALNPLRKC |
| | | QKRPCGHPGDTPFGTFTLTGGNVFEYGVKA |
| | | VYTCNEGYQLLGEINYRECDTDGWTNDIPI |
| | | CEVVKCLPVTAPENGKIVSSAMEPDREYHF |
| | | GQAVRFVCNSGYKIEGDEEMHCSDDGFWSK |
| | | EKPKCVEISCKSPDVINGSPISQKIIYKEN |
| | | ERFQYKCNMGYEYSERGDAVCTESGWRPLP |
| | | SCEEKSCDNPYIPNGDYSPLRIKHRTGDEI |
| | | TYQCRNGFYPATRGNTAKCTSTGWIPAPRC |
| | | TLK |
| Compound 2 | 100 | EPKSADKTHTCPPCPAPELLGGPSVFLFPP |
| | | KPKDTLMISRTPEVTCVVVDVSHEDPEVKF |
| | | NWYVDGVEVHNAKTKPREEQYNSTYRVVSV |
| | | LTVLHQDWLNGKEYKCKVSNKALPAPIEKT |
| | | ISKAKGQPREPQVYTLPPSRDELTKNQVSL |
| | | TCLVKGFYPSDIAVEWESNGQPENNYKTTP |
| | | PVLDSDGSFFLYSKLTVDKSRWQQGNVFSC |
| | | SVMHEALHNHYTQKSLSLSPGKGGGGAGGG |
| | | GAGGGGSEDCNELPPRRNTEILTGSWSDQT |
| | | YPEGTQAIYKCRPGYRSLGNVIMVCRKGEW |
| | | VALNPLRKCQKRPCGHPGDTPFGTFTLTGG |
| | | NVFEYGVKAVYTCNEGYQLLGEINYRECDT |
| | | DGWTNDIPICEVVKCLPVTAPENGKIVSSA |
| | | MEPDREYHFGQAVRFVCNSGYKIEGDEEMH |
| | | CSDDGFWSKEKPKCVEISCKSPDVINGSPI |
| | | SQKIIYKENERFQYKCNMGYEYSERGDAVC |
| | | TESGWRPLPSCEEKSCDNPYIPNGDYSPLR |
| | | IKHRTGDEITYQCRNGFYPATRGNTAKCTS |
| | | TGWIPAPRCTLK |
| Compound 3 | 101 | EPKSADKTHTCPPCPAPELLGGPSVFLFPP |
| | | KPKDTLMISRTPEVTCVVVDVSHEDPEVKF |
| | | NWYVDGVEVHNAKTKPREEQYNSTYRVVSV |
| | | LTVLHQDWLNGKEYKCKVSNKALPAPIEKT |
| | | ISKAKGQPREPQVYTLPPSRDELTKNQVSL |
| | | TCLVKGFYPSDIAVEWESNGQPENNYKTTP |
| | | PVLDSDGSFFLYSKLTVDKSRWQQGNVFSC |
| | | SVMHEALHNHYTQKSLSLSPGKEDCNELPP |
| | | RRNTEILTGSWSDQTYPEGTQAIYKCRPGY |
| | | RSLGNVIMVCRKGEWVALNPLRKCQKRPCG |
| | | HPGDTPFGTFTLTGGNVFEYGVKAVYTCNE |
| | | GYQLLGEINYRECDTDGWTNDIPICEVVKC |
| | | LPVTAPENGKIVSSAMEPDREYHFGQAVRF |
| | | VCNSGYKIEGDEEMHCSDDGFWSKEKPKCV |
| | | EISCKSPDVINGSPISQKIIYKENERFQYK |
| | | CNMGYEYSERGDAVCTESGWRPLPSCEEKS |
| | | CDNPYIPNGDYSPLRIKHRTGDEITYQCRN |
| | | GFYPATRGNTAKCTSTGWIPAPRCTLK |
| Compound 4 | 102 | EDCNELPPRRNTEILTGSWSDQTYPEGTQA |
| | | IYKCRPGYRSLGNVIMVCRKGEWVALNPLR |
| | | KCQKRPCGHPGDTPFGTFTLTGGNVFEYGV |
| | | KAVYTCNEGYQLLGEINYRECDTDGWTNDI |
| | | PICEVVKCLPVTAPENGKIVSSAMEPDREY |
| | | HFGQAVRFVCNSGYKIEGDEEMHCSDDGFW |
| | | SKEKPKCVEISCKSPDVINGSPISQKIIYK |
| | | ENERFQYKCNMGYEYSERGDAVCTESGWRP |
| | | LPSCEEKSCDNPYIPNGDYSPLRIKHRTGD |
| | | EITYQCRNGFYPATRGNTAKCTSTGWIPAP |
| | | RCTLKGGGGAGGGGAGGGGSDKTHTCPPCP |
| | | APELLGGPSVFLFPPKPKDTLMISRTPEVT |
| | | CVVVDVSHEDPEVKFNWYVDGVEVHNAKTK |
| | | PREEQYNSTYRVVSVLTVLHQDWLNGKEYK |
| | | CKVSNKALPAPIEKTISKAKGQPREPQVYT |
| | | LPPSRDELTKNQVSLTCLVKGFYPSDIAVE |
| | | WESNGQPENNYKTTPPVLDSDGSFFLYSKL |
| | | TVDKSRWQQGNVFSCSVMHEALHNHYTQKS |
| | | LSLSPGK |
| Compound 7 | 103 | CVECPPCPAPPVAGPSVFLFPPKPKDTLMI |
| | | SRTPEVTCVVVDVSQEDPEVQFNWYVDGVE |
| | | VHNAKTKPREEQFNSTYRVVSVLTVLHQDW |
| | | LNGKEYKCKVSNKGLPSSIEKTISKAKGQP |
| | | REPQVYTLPPSQEEMTKNQVSLTCLVKGFY |
| | | PSDIAVEWESNGQPENNYKTTPPVLDSDGS |
| | | FFLYSRLTVDKSRWQEGNVFSCSVMHEALH |
| | | NHYTQKSLSLSLGKGGGGAGGGGAGGGAGG |
| | | GGSEDCNELPPRRNTEILTGSWSDQTYPEG |
| | | TQAIYKCRPGYRSLGNVIMVCRKGEWVALN |
| | | PLRKCQKRPCGHPGDTPFGTFTLTGGNVFE |
| | | YGVKAVYTCNEGYQLLGEINYRECDTDGWT |
| | | NDIPICEVVKCLPVTAPENGKIVSSAMEPD |
| | | REYHFGQAVRFVCNSGYKIEGDEEMHCSDD |
| | | GFWSKEKPKCVEISCKSPDVINGSPISQKI |
| | | IYKENERFQYKCNMGYEYSERGDAVCTESG |
| | | WRPLPSCEEKS |
| Compound 8 | 104 | CVECPPCPAPPVAGPSVFLFPPKPKDTLMI |
| | | SRTPEVTCVVVDVSQEDPEVQFNWYVDGVE |
| | | VHNAKTKPREEQFNSTYRVVSVLTVLHQDW |
| | | LNGKEYKCKVSNKGLPSSIEKTISKAKGQP |
| | | REPQVYTLPPSQEEMTKNQVSLTCLVKGFY |
| | | PSDIAVEWESNGQPENNYKTTPPVLDSDGS |
| | | FFLYSRLTVDKSRWQEGNVFSCSVMHEALH |
| | | NHYTQKSLSLSLGKGGGGAGGGGAGGGAGG |
| | | GGSKEDCNELPPRRNTEILTGSWSDQTYPE |
| | | GTQAIYKCRPGYRSLGNVIMVCRKGEWVAL |
| | | NPLRKCQKRPCGHPGDTPFGTFTLTGGNVF |
| | | EYGVKAVYTCNEGYQLLGEINYRECDTDGW |
| | | TNDIPICEVVKCLPVTAPENGKIVSSAMEP |
| | | DREYHFGQAVRFVCNSGYKIEGDEEMHCSD |
| | | DGFWSKEKPKCVEISCKSPDVINGSPISQK |
| | | IIYKENERFQYKCNMGYEYSERGDAVCTES |
| | | GWRPLPSCEEKS |
| Compound 9 | 105 | CVECPPCPAPPVAGPSVFLFPPKPKDTLMI |
| | | SRTPEVTCVVVDVSQEDPEVQFNWYVDGVE |
| | | VHNAKTKPREEQFNSTYRVVSVLTVLHQDW |
| | | LNGKEYKCKVSNKGLPSSIEKTISKAKGQP |
| | | REPQVYTLPPSQEEMTKNQVSLTCLVKGFY |
| | | PSDIAVEWESNGQPENNYKTTPPVLDSDGS |
| | | FFLYSRLTVDKSRWQEGNVFSCSVMHEALH |
| | | NHYTQKSLSLSLGKGGGGAGGGGAGGGAGG |
| | | GGSREDCNELPPRRNTEILTGSWSDQTYPE |
| | | GTQAIYKCRPGYRSLGNVIMVCRKGEWVAL |
| | | NPLRKCQKRPCGHPGDTPFGTFTLTGGNVF |
| | | EYGVKAVYTCNEGYQLLGEINYRECDTDGW |
| | | TNDIPICEVVKCLPVTAPENGKIVSSAMEP |
| | | DREYHFGQAVRFVCNSGYKIEGDEEMHCSD |
| | | DGFWSKEKPKCVEISCKSPDVINGSPISQK |
| | | IIYKENERFQYKCNMGYEYSERGDAVCTES |
| | | GWRPLPSCEEKS |
| Compound 10 | 106 | CVECPPCPAPPVAGPSVFLFPPKPKDTLMI |
| | | SRTPEVTCVVVDVSQEDPEVQFNWYVDGVE |
| | | VHNAKTKPREEQFNSTYRVVSVLTVLHQDW |
| | | LNGKEYKCKVSNKGLPSSIEKTISKAKGQP |
| | | REPQVYTLPPSQEEMTKNQVSLTCLVKGFY |
| | | PSDIAVEWESNGQPENNYKTTPPVLDSDGS |
| | | FFLYSRLTVDKSRWQEGNVFSCSVMHEALH |
| | | NHYTQKSLSLSLGKGGGGAGGGAGGGGSKE |
| | | DCNELPPRRNTEILTGSWSDQTYPEGTQAI |
| | | YKCRPGYRSLGNVIMVCRKGEWVALNPLRK |
| | | CQKRPCGHPGDTPFGTFTLTGGNVFEYGVK |
| | | AVYTCNEGYQLLGEINYRECDTDGWTNDIP |
| | | ICEVVKCLPVTAPENGKIVSSAMEPDREYH |
| | | FGQAVRFVCNSGYKIEGDEEMHCSDDGFWS |
| | | KEKPKCVEISCKSPDVINGSPISQKIIYKE |
| | | NERFQYKCNMGYEYSERGDAVCTESGWRPL |
| | | PSCEEKS |
| Compound 11 | 107 | CVECPPCPAPPVAGPSVFLFPPKPKDTLMI |
| | | SRTPEVTCVVVDVSQEDPEVQFNWYVDGVE |
| | | VHNAKTKPREEQFNSTYRVVSVLTVLHQDW |
| | | LNGKEYKCKVSNKGLPSSIEKTISKAKGQP |
| | | REPQVYTLPPSQEEMTKNQVSLTCLVKGFY |
| | | PSDIAVEWESNGQPENNYKTTPPVLDSDGS |
| | | FFLYSRLTVDKSRWQEGNVFSCSVMHEALH |
| | | NHYTQKSLSLSLGKGGGGAGGGAGGGGSRE |
| | | DCNELPPRRNTEILTGSWSDQTYPEGTQAI |
| | | YKCRPGYRSLGNVIMVCRKGEWVALNPLRK |
| | | CQKRPCGHPGDTPFGTFTLTGGNVFEYGVK |
| | | AVYTCNEGYQLLGEINYRECDTDGWTNDIP |
| | | ICEVVKCLPVTAPENGKIVSSAMEPDREYH |
| | | FGQAVRFVCNSGYKIEGDEEMHCSDDGFWS |
| | | KEKPKCVEISCKSPDVINGSPISQKIIYKE |
| | | NERFQYKCNMGYEYSERGDAVCTESGWRPL |
| | | PSCEEKS |

TABLE 3
DNA sequences for FH-Fc fusion
proteins described in the Examples

| | SEQ | |
|---|---|---|
| | ID | |
| Compound | NO. | DNA Sequence |
| Linker | 119 | GGCGGAGGCGGAGCTGGTGGTGGTG |
| used in | | CTGGCGGCGGAGGA |
| Compound 10 | | |
| and | | |
| Compound 11 | | |
| Compound 1 | 118 | GATAAGAAAATCGTGCCCAGAGACT |
| | | GCGGCTGCAAGCCCTGTATCTGTAC |
| | | AGTGCCTGAGGTGTCCAGCGTGTTC |
| | | ATCTTCCCACCTAAGCCTAAGGACG |
| | | TGCTGACCATCACACTGACCCCTAA |
| | | AGTGACCTGCGTGGTGGTGGACATC |
| | | AGCAAGGATGACCCTGAGGTGCAGT |
| | | TCAGTTGGTTCGTGGACGACGTGGA |
| | | AGTGCACACAGCCCAGACCAAGCCT |
| | | AGAGAGGAACAGATCAACAGCACCT |
| | | TCAGAAGCGTGTCCGAGCTGCCCAT |
| | | CATGCACCAGGACTGGCTGAACGGC |
| | | AAAGAATTCAAGTGCAGAGTGAACA |
| | | GCGCCGCCTTTCCTGCTCCTATCGA |
| | | GAAAACCATCTCCAAGACCAAGGGC |
| | | AGACCCAAGGCTCCCCAGGTGTACA |
| | | CAATCCCTCCACCTAAAGAACAGAT |
| | | GGCCAAGGACAAGGTGTCCCTGACC |
| | | TGCATGATCACCAATTTCTTCCCAG |
| | | AGGACATCACCGTGGAATGGCAGTG |
| | | GAATGGACAGCCCGCCGAGAACTAC |
| | | AAGAACACCCAGCCTATCATGGACA |
| | | CCGACGGCAGCTACTTCGTGTACAG |
| | | CAAGCTGAACGTGCAGAAGTCCAAC |
| | | TGGGAGGCCGGCAACACCTTTACCT |
| | | GTTCTGTGCTGCACGAGGGCCTGCA |
| | | CAACCACCACACAGAGAAGTCTCTG |
| | | TCTCACAGCCCTGGAAAAGGCGGAG |
| | | GCGGAGCTGGTGGTGGCGGAGCAGG |
| | | CGGCGGTGCTGGCGGCGGAGGATCT |
| | | GAAGATTGCAAAGGACCTCCTCCAA |
| | | GAGAGAACAGCGAGATCCTGTCTGG |
| | | CTCTTGGAGCGAGCAGCTGTATCCT |
| | | GAGGGAACCCAGGCCACCTACAAGT |
| | | GCAGGCCTGGCTATAGAACCCTGGG |
| | | CACCATCGTGAAAGTGTGCAAGAAT |
| | | GGCAAATGGGTCGCCAGCAATCCCA |
| | | GCCGGATCTGCAGAAAGAAACCTTG |
| | | CGGACACCCCGGCGATACCCCTTTC |
| | | GGATCTTTTAGACTGGCCGTGGGCA |
| | | GCCAGTTTGAGTTCGGAGCCAAGGT |
| | | GGTGTATACCTGCGACGATGGCTAT |
| | | CAGCTGCTGGGCGAGATCGACTATA |
| | | GAGAGTGTGGCGCCGACGGCTGGAT |
| | | CAACGATATCCCTCTGTGCGAGGTG |
| | | GTCAAGTGCCTGCCTGTGACAGAGC |
| | | TGGAAAACGGCAGAATTGTGTCCGG |
| | | CGCTGCCGAGACAGACCAAGAGTAC |
| | | TACTTTGGCCAGGTCGTCAGATTCG |
| | | AGTGCAACAGCGGCTTCAAGATCGA |
| | | GGGCCACAAAGAGATCCACTGCAGC |
| | | GAGAACGGCCTGTGGTCCAACGAGA |
| | | AGCCCAGATGCGTGGAAATCCTGTG |
| | | CACCCCTCCTAGAGTGGAAAATGGC |
| | | GACGGCATCAACGTGAAGCCCGTGT |
| | | ACAAAGAGAACGAGCGCTACCACTA |
| | | TAAGTGCAAGCACGGCTACGTGCCC |
| | | AAAGAACGGGGAGATGCCGTGTGTA |
| | | CAGGCAGCGGATGGTCTAGCCAGCC |
| | | TTTCTGCGAAGAGAAGAGATG |
| Compound 5 | 110 | GTGGAATGCCCTCCATGTCCTGCTC |
| | | CTCCAGTGGCCGGACCTTCCGTGTT |
| | | TCTGTTCCCTCCAAAGCCTAAGGAC |
| | | ACCCTGATGATCAGCAGAACCCCTG |
| | | AAGTGACCTGCGTGGTGGTGGACGT |
| | | TTCCCAAGAGGATCCCGAGGTGCAG |
| | | TTCAATTGGTACGTGGACGGCGTGG |
| | | AAGTGCACAACGCCAAGACCAAGCC |
| | | TAGAGAGGAACAGTTCAACAGCACC |
| | | TACAGAGTGGTGTCCGTGCTGACCG |
| | | TGCTGCACCAGGATTGGCTGAACGG |
| | | CAAAGAGTACAAGTGCAAGGTGTCC |
| | | AACAAGGGCCTGCCTAGCAGCATCG |
| | | AGAAAACCATCAGCAAGGCCAAGGG |
| | | CCAGCCAAGAGAACCCCAGGTTTAC |
| | | ACCCTGCCTCCAAGCCAAGAGGAAA |
| | | TGACCAAGAACCAGGTGTCCCTGAC |
| | | CTGCCTGGTCAAGGGCTTCTACCCT |
| | | TCCGATATCGCTGTGGAATGGGAGA |
| | | GCAACGGCCAGCCTGAGAACAACTA |
| | | CAAGACCACACCTCCTGTGCTGGAC |
| | | AGCGACGGCAGCTTTTTTCTGTACT |
| | | CCCGCCTGACCGTGGACAAGAGCAG |
| | | ATGGCAAGAGGGCAACGTGTTCAGC |
| | | TGCTCTGTGATGCACGAGGCCCTGC |
| | | ACAACCACTACACCCAGAAGTCTCT |
| | | GAGCCTGTCTCTCGGAAAAGGCGGA |
| | | GGCGGAGCTGGTGGTGGCGGAGCAG |
| | | GCGGCGGAGGATCTGAAGATTGCAA |
| | | TGAGCTGCCTCCTCGGCGGAACACA |
| | | GAGATCTTGACAGGCTCTTGGAGCG |
| | | ACCAGACATACCCTGAGGGCACCCA |
| | | GGCCATCTACAAGTGTAGACCTGGC |
| | | TACCGCAGCCTGGGCAATGTGATCA |
| | | TGGTCTGCAGAAAAGGCGAGTGGGT |
| | | CGCCCTGAATCCTCTGAGAAAGTGC |
| | | CAGAAGAGGCCTTGCGGACACCCCG |
| | | GCGATACACCTTTTGGCACATTCAC |
| | | CCTGACCGGCGGCAATGTGTTTGAG |
| | | TATGGCGTGAAGGCCGTGTACACCT |
| | | GTAACGAGGGATATCAGCTGCTGGG |
| | | CGAGATCAACTACAGAGAGTGTGAT |
| | | ACCGACGGCTGGACCAACGACATCC |
| | | CTATCTGCGAGGTGGTCAAGTGCCT |
| | | GCCTGTGACAGCCCCTGAGAATGGC |
| | | AAGATCGTGTCCAGCGCCATGGAAC |
| | | CCGACAGAGAGTATCACTTTGGCCA |
| | | GGCCGTCAGATTCGTGTGCAACAGC |
| | | GGCTATAAGATCGAGGGCGACGAGG |
| | | AAATGCACTGCAGCGACGACGGCTT |
| | | CTGGTCCAAAGAAAAGCCCAAATGC |
| | | GTGGAAATCAGCTGCAAGAGCCCCG |
| | | ACGTGATCAACGGCAGCCCTATCAG |
| | | CCAGAAGATCATCTACAAAGAGAAC |
| | | GAGCGGTTCCAGTATAAGTGCAACA |
| | | TGGGCTACGAGTACAGCGAGCGGGG |
| | | AGATGCCGTGTGTACAGAATCTGGA |
| | | TGGCGGCCTCTGCCTAGCTGCGAGG |
| | | AAAAGAGCTGCGACAACCCTTACAT |
| | | CCCCAACGGCGACTACAGCCCTCTG |
| | | CGGATTAAGCACAGAACCGGCGACG |
| | | AGATCACCTACCAGTGCAGAAACGG |
| | | CTTTTACCCCGCCACCAGAGGCAAT |
| | | ACCGCCAAG |
| Compound 2 | 111 | GAACCGAAGTCAGCTGACAAGACCC |
| | | ACACTTGCCCTCCATGCCCTGCCCC |
| | | TGAACTGCTTGGGGGCCTTCCGTGT |
| | | TCCTGTTCCCCCCGAAACCTAAAGA |
| | | TACCCTCATGATCTCGCGAACCCCG |
| | | GAAGTGACTTGCGTGGTCGTGGATG |
| | | TGTCCCACGAGGATCCTGAAGTGAA |
| | | GTTCAATTGGTACGTGGATGGAGTG |
| | | GAAGTCCATAACGCTAAGACGAAGC |
| | | CGAGAGAGGAACAGTACAACTCGAC |
| | | CTACCGCGTGGTGTCCGTGCTCACC |
| | | GTGCTGCACCAAGACTGGCTGAACG |
| | | GAAAGGAATACAAGTGTAAAGTGTC |
| | | CAACAAGGCCTTGCCAGCCCCTATC |
| | | GAAAAGACCATATCAAAAGCAAAGG |
| | | GACAGCCCAGAGAGCCCCAGGTGTA |
| | | CACCCTGCCACCTTCCCGGGATGAG |
| | | CTGACCAAGAACCAAGTCTCCCTGA |
| | | CCTGTCTGGTCAAGGGATTCTACCC |
| | | CTCCGATATCGCGGTCGAATGGGAG |
| | | AGCAACGGACAACCCGAAAACAACT |
| | | ACAAGACTACCCCTCCCGTCCTCGA |
| | | CTCCGATGGCTCGTTCTTCCTGTAT |
| | | TCGAAGTTGACTGTGGACAAGTCCA |
| | | GATGGCAGCAGGGCAACGTGTTCAG |
| | | CTGCAGCGTGATGCACGAGGCGCTG |
| | | CACAATCATTACACCCAAAAGTCCC |
| | | TGTCCTTGAGCCCTGGAAAGGGGGG |
| | | AGGAGGTGCAGGAGGAGGAGGCGCA |
| | | GGAGGAGGAGGTTCGGAGGACTGCA |
| | | ACGAGCTTCCACCGCGGAGAAATAC |
| | | TGAAATTCTGACAGGCTCATGGTCT |
| | | GATCAGACTTACCCGGAAGGCACCC |
| | | AGGCCATCTACAAATGTCGGCCCGG |
| | | CTACAGGTCCCTCGGAAACGTGATC |
| | | ATGGTCTGCAGGAAGGGGGAATGGG |
| | | TCGCCCTGAACCCGCTGAGAAAGTG |
| | | CCAGAAGCGGCCATGTGGACACCCG |
| | | GGAGACACTCCCTTCGGCACCTTTA |
| | | CCCTGACCGGTGGAAACGTGTTCGA |
| | | ATACGGCGTGAAGGCCGTGTACACT |
| | | TGCAACGAAGGATATCAGCTTCTCG |
| | | GCGAGATCAACTATCGGGAATGCGA |
| | | CACCGATGGCTGGACCAACGACATC |
| | | CCTATCTGCGAAGTCGTCAAGTGTC |
| | | TCCCTGTGACTGCCCCGGAAAACGG |
| | | AAAGATCGTGTCCTCCGCCATGGAA |
| | | CCTGACCGGGAATACCACTTTGGCC |
| | | AAGCCGTGCGGTTCGTGTGCAACAG |
| | | CGGCTACAAAATTGAAGGAGATGAA |
| | | GAAATGCATTGTAGCGATGACGGCT |
| | | TCTGGTCCAAGGAGAAGCCTAAGTG |
| | | CGTGGAAATTAGCTGCAAGTCCCCC |
| | | GACGTGATCAACGGTTCCCCCATCT |
| | | CCCAAAAGATTATCTACAAGGAGAA |
| | | CGAGCGCTTCCAGTACAAGTGCAAC |
| | | ATGGGATACGAGTACAGCGAGAGAG |
| | | GGGACGCGGTCTGCACCGAGTCCGG |
| | | GTGGAGGCCTCTGCCGTCATGCGAA |
| | | GAAAAGAGCTGCGACAACCCCTACA |
| | | TTCCGAACGGAGACTACAGCCCGCT |
| | | CAGGATCAAGCACCGCACCGGGGAT |
| | | GAAATCACTTACCAATGCCGCAACG |
| | | GATTCTATCCAGCGACTCGCGGGAA |
| | | TACCGCCAAATGCACCTCGACTGGT |
| | | TGGATTCCGGCCCCAAGGTGCACCC |
| | | TGAAG |
| Compound 3 | 112 | GAACCGAAGTCAGCTGACAAGACCC |
| | | ACACTTGCCCTCCATGCCCTGCCCC |
| | | TGAACTGCTTGGGGGGCCTTCCGTG |
| | | TTCCTGTTCCCCCCGAAACCTAAAG |
| | | ATACCCTCATGATCTCGCGAACCCC |
| | | GGAAGTGACTTGCGTGGTCGTGGAT |
| | | GTGTCCCACGAGGATCCTGAAGTGA |
| | | AGTTCAATTGGTACGTGGATGGAGT |
| | | GGAAGTCCATAACGCTAAGACGAAG |
| | | CCGAGAGAGGAACAGTACAACTCGA |
| | | CCTACCGCGTGGTGTCCGTGCTCAC |
| | | CGTGCTGCACCAAGACTGGCTGAAC |
| | | GGAAAGGAATACAAGTGTAAAGTGT |
| | | CCAACAAGGCCTTGCCAGCCCCTAT |
| | | CGAAAAGACCATATCAAAAGCAAAG |
| | | GGACAGCCCAGAGAGCCCCAGGTGT |
| | | ACACCCTGCCACCTTCCCGGGATGA |
| | | GCTGACCAAGAACCAAGTCTCCCTG |
| | | ACCTGTCTGGTCAAGGGATTCTACC |
| | | CCTCCGATATCGCGGTCGAATGGGA |
| | | GAGCAACGGACAACCCGAAAACAAC |
| | | TACAAGACTACCCCTCCCGTCCTCG |
| | | ACTCCGATGGCTCGTTCTTCCTGTA |
| | | TTCGAAGTTGACTGTGGACAAGTCC |
| | | AGATGGCAGCAGGGCAACGTGTTCA |
| | | GCTGCAGCGTGATGCACGAGGCGCT |
| | | GCACAATCATTACACCCAAAAGTCC |
| | | CTGTCCTTGAGCCCTGGAAAGGAGG |
| | | ACTGCAACGAGCTTCCACCGCGGAG |
| | | AAATACTGAAATTCTGACAGGCTCA |
| | | TGGTCTGATCAGACTTACCCGGAAG |
| | | GCACCCAGGCCATCTACAAATGTCG |
| | | GCCCGGCTACAGGTCCCTCGGAAAC |
| | | GTGATCATGGTCTGCAGGAAGGGGG |
| | | AATGGGTCGCCCTGAACCCGCTGAG |
| | | AAAGTGCCAGAAGCGGCCATGTGGA |
| | | CACCCGGGAGACACTCCCTTCGGCA |
| | | CCTTTACCCTGACCGGTGGAAACGT |
| | | GTTCGAATACGGCGTGAAGGCCGTG |
| | | TACACTTGCAACGAAGGATATCAGC |
| | | TTCTCGGCGAGATCAACTATCGGGA |
| | | ATGCGACACCGATGGCTGGACCAAC |
| | | GACATCCCTATCTGCGAAGTCGTCA |
| | | AGTGTCTCCCTGTGACTGCCCCGGA |
| | | AAACGGAAAGATCGTGTCCTCCGCC |
| | | ATGGAACCTGACCGGGAATACCACT |
| | | TTGGCCAAGCCGTGCGGTTCGTGTG |
| | | CAACAGCGGCTACAAAATTGAAGGA |
| | | GATGAAGAAATGCATTGTAGCGATG |
| | | ACGGCTTCTGGTCCAAGGAGAAGCC |
| | | TAAGTGCGTGGAAATTAGCTGCAAG |
| | | TCCCCCGACGTGATCAACGGTTCCC |
| | | CCATCTCCCAAAAGATTATCTACAA |
| | | GGAGAACGAGCGCTTCCAGTACAAG |
| | | TGCAACATGGGATACGAGTACAGCG |
| | | AGAGAGGGGACGCGGTCTGCACCGA |
| | | GTCCGGGTGGAGGCCTCTGCCGTCA |
| | | TGCGAAGAAAAGAGCTGCGACAACC |
| | | CCTACATTCCGAACGGAGACTACAG |
| | | CCCGCTCAGGATCAAGCACCGCACC |
| | | GGGGATGAAATCACTTACCAATGCC |
| | | GCAACGGATTCTATCCAGCGACTCG |
| | | CGGGAATACCGCCAAATGCACCTCG |
| | | ACTGGTTGGATTCCGGCCCCAAGGT |
| | | GCACCCTGAAG |
| Compound 7 | 113 | GAATGTCCTCCTTGTCCTGCTCCTC |
| | | CAGTGGCCGGACCTTCCGTGTTTCT |
| | | GTTCCCTCCAAAGCCTAAGGACACC |
| | | CTGATGATCAGCAGAACCCCTGAAG |
| | | TGACCTGCGTGGTGGTGGACGTTTC |
| | | CCAAGAGGATCCCGAGGTGCAGTTC |
| | | AATTGGTACGTGGACGGCGTGGAAG |
| | | TGCACAACGCCAAGACCAAGCCTAG |
| | | AGAGGAACAGTTCAACAGCACCTAC |
| | | AGAGTGGTGTCCGTGCTGACCGTGC |
| | | TGCACCAGGATTGGCTGAACGGCAA |
| | | AGAGTACAAGTGCAAGGTGTCCAAC |
| | | AAGGGCCTGCCTAGCAGCATCGAGA |
| | | AAACCATCAGCAAGGCCAAGGGCCA |
| | | GCCAAGAGAACCCCAGGTTTACACC |
| | | CTGCCTCCAAGCCAAGAGGAAATGA |
| | | CCAAGAACCAGGTGTCCCTGACCTG |
| | | CCTGGTCAAGGGCTTCTACCCTTCC |
| | | GATATCGCCGTGGAATGGGAGAGCA |
| | | ATGGCCAGCCTGAGAACAACTACAA |
| | | GACCACACCTCCTGTGCTGGACAGC |
| | | GACGGCAGCTTTTTTCTGTACTCCC |
| | | GCCTGACCGTGGACAAGAGCAGATG |
| | | GCAAGAGGGCAACGTGTTCAGCTGC |
| | | TCTGTGATGCACGAGGCCCTGCACA |
| | | ACCACTACACCCAGAAGTCTCTGAG |
| | | CCTGTCTCTCGGAAAAGGCGGAGGC |
| | | GGAGCTGGTGGTGGCGGAGCAGGCG |
| | | GCGGTGCTGGCGGCGGAGGATCTGA |
| | | AGATTGCAATGAGCTGCCTCCTCGG |
| | | CGGAACACAGAGATCTTGACAGGCT |
| | | CTTGGAGCGACCAGACATACCCTGA |
| | | GGGCACCCAGGCCATCTACAAGTGT |
| | | AGACCTGGCTACCGCAGCCTGGGCA |
| | | ATGTGATCATGGTCTGCAGAAAAGG |
| | | CGAGTGGGTCGCCCTGAATCCTCTG |
| | | AGAAAGTGCCAGAAGAGGCCTTGCG |
| | | GACACCCCGGCGATACACCTTTTGG |
| | | CACATTCACCCTGACCGGCGGCAAT |
| | | GTGTTTGAGTATGGCGTGAAGGCCG |
| | | TGTACACCTGTAACGAGGGATATCA |
| | | GCTGCTGGGCGAGATCAACTACAGA |
| | | GAGTGTGATACCGACGGCTGGACCA |
| | | ACGACATCCCTATCTGCGAGGTGGT |
| | | CAAGTGCCTGCCTGTGACAGCCCCT |
| | | GAGAATGGCAAGATCGTGTCCAGCG |
| | | CCATGGAACCCGACAGAGAGTATCA |
| | | CTTTGGCCAGGCCGTCAGATTCGTG |
| | | TGCAACAGCGGCTATAAGATCGAGG |
| | | GCGACGAGGAAATGCACTGCAGCGA |
| | | CGACGGCTTCTGGTCCAAAGAAAAG |
| | | CCCAAATGCGTGGAAATCAGCTGCA |
| | | AGAGCCCCGACGTGATCAACGGCAG |
| | | CCCTATCAGCCAGAAGATCATCTAC |
| | | AAAGAGAACGAGCGGTTCCAGTATA |
| | | AGTGCAACATGGGCTACGAGTACAG |
| | | CGAGCGGGGAGATGCCGTGTGTACA |
| | | GAATCTGGATGGCGGCCTCTGCCTA |
| | | GCTGCGAGGAAAAGTCT |
| Compound 8 | 114 | GAATGTCCTCCTTGTCCTGCTCCTC |
| | | CAGTGGCCGGACCTTCCGTGTTTCT |
| | | GTTCCCTCCAAAGCCTAAGGACACC |
| | | CTGATGATCAGCAGAACCCCTGAAG |
| | | TGACCTGCGTGGTGGTGGACGTTTC |
| | | CCAAGAGGATCCCGAGGTGCAGTTC |
| | | AATTGGTACGTGGACGGCGTGGAAG |
| | | TGCACAACGCCAAGACCAAGCCTAG |
| | | AGAGGAACAGTTCAACAGCACCTAC |
| | | AGAGTGGTGTCCGTGCTGACCGTGC |
| | | TGCACCAGGATTGGCTGAACGGCAA |
| | | AGAGTACAAGTGCAAGGTGTCCAAC |
| | | AAGGGCCTGCCTAGCAGCATCGAGA |
| | | AAACCATCAGCAAGGCCAAGGGCCA |
| | | GCCAAGAGAACCCCAGGTTTACACC |
| | | CTGCCTCCAAGCCAAGAGGAAATGA |
| | | CCAAGAACCAGGTGTCCCTGACCTG |
| | | CCTGGTCAAGGGCTTCTACCCTTCC |
| | | GATATCGCCGTGGAATGGGAGAGCA |
| | | ATGGCCAGCCTGAGAACAACTACAA |
| | | GACCACACCTCCTGTGCTGGACAGC |
| | | GACGGCAGCTTTTTTCTGTACTCCC |
| | | GCCTGACCGTGGACAAGAGCAGATG |
| | | GCAAGAGGGCAACGTGTTCAGCTGC |
| | | TCTGTGATGCACGAGGCCCTGCACA |
| | | ACCACTACACCCAGAAGTCTCTGAG |
| | | CCTGTCTCTCGGAAAAGGCGGAGGC |
| | | GGAGCTGGTGGTGGCGGAGCAGGCG |
| | | GCGGTGCTGGCGGCGGAGGATCTAA |
| | | AGAAGATTGCAACGAGCTGCCTCCT |
| | | CGGCGGAATACCGAGATTCTGACAG |
| | | GCTCTTGGAGCGACCAGACATACCC |
| | | TGAGGGCACCCAGGCCATCTACAAG |
| | | TGTAGACCTGGCTACCGCAGCCTGG |
| | | GCAATGTGATCATGGTCTGCAGAAA |
| | | AGGCGAGTGGGTCGCCCTGAATCCT |
| | | CTGAGAAAGTGCCAGAAGAGGCCTT |
| | | GCGGACACCCCGGCGATACACCTTT |
| | | TGGCACATTCACCCTGACCGGCGGC |
| | | AATGTGTTTGAGTATGGCGTGAAGG |
| | | CCGTGTACACCTGTAACGAGGGATA |
| | | TCAGCTGCTGGGCGAGATCAACTAC |
| | | AGAGAGTGTGATACCGACGGCTGGA |
| | | CCAACGACATCCCTATCTGCGAGGT |
| | | GGTCAAGTGCCTGCCTGTGACAGCC |
| | | CCTGAGAATGGCAAGATCGTGTCCA |
| | | GCGCCATGGAACCCGACAGAGAGTA |
| | | TCACTTTGGCCAGGCCGTCAGATTC |
| | | GTGTGCAACAGCGGCTATAAGATCG |
| | | AGGGCGACGAGGAAATGCACTGCAG |
| | | CGACGACGGCTTCTGGTCCAAAGAA |
| | | AAGCCCAAATGCGTGGAAATCAGCT |
| | | GCAAGAGCCCCGACGTGATCAACGG |
| | | CAGCCCTATCAGCCAGAAGATCATC |
| | | TACAAAGAGAACGAGCGGTTCCAGT |
| | | ATAAGTGCAACATGGGCTACGAGTA |
| | | CAGCGAGCGGGGAGATGCCGTGTGT |
| | | ACAGAATCTGGATGGCGGCCTCTGC |
| | | CTAGCTGCGAGGAAAAGTCT |
| Compound 9 | 115 | GAATGTCCTCCTTGTCCTGCTCCTC |
| | | CAGTGGCCGGACCTTCCGTGTTTCT |
| | | GTTCCCTCCAAAGCCTAAGGACACC |
| | | CTGATGATCAGCAGAACCCCTGAAG |
| | | TGACCTGCGTGGTGGTGGACGTTTC |
| | | CCAAGAGGATCCCGAGGTGCAGTTC |
| | | AATTGGTACGTGGACGGCGTGGAAG |
| | | TGCACAACGCCAAGACCAAGCCTAG |
| | | AGAGGAACAGTTCAACAGCACCTAC |
| | | AGAGTGGTGTCCGTGCTGACCGTGC |
| | | TGCACCAGGATTGGCTGAACGGCAA |
| | | AGAGTACAAGTGCAAGGTGTCCAAC |
| | | AAGGGCCTGCCTAGCAGCATCGAGA |
| | | AAACCATCAGCAAGGCCAAGGGCCA |
| | | GCCAAGAGAACCCCAGGTTTACACC |
| | | CTGCCTCCAAGCCAAGAGGAAATGA |
| | | CCAAGAACCAGGTGTCCCTGACCTG |
| | | CCTGGTCAAGGGCTTCTACCCTTCC |
| | | GATATCGCCGTGGAATGGGAGAGCA |
| | | ATGGCCAGCCTGAGAACAACTACAA |
| | | GACCACACCTCCTGTGCTGGACAGC |
| | | GACGGCAGCTTTTTTCTGTACTCCC |
| | | GCCTGACCGTGGACAAGAGCAGATG |
| | | GCAAGAGGGCAACGTGTTCAGCTGC |
| | | TCTGTGATGCACGAGGCCCTGCACA |
| | | ACCACTACACCCAGAAGTCTCTGAG |
| | | CCTGTCTCTCGGAAAAGGCGGAGGC |
| | | GGAGCTGGTGGTGGCGGAGCAGGCG |
| | | GCGGTGCTGGCGGCGGAGGATCTCG |
| | | GGAAGATTGCAACGAGCTGCCTCCT |
| | | CGGCGGAATACCGAGATTCTGACAG |
| | | GCTCTTGGAGCGACCAGACATACCC |
| | | TGAGGGCACCCAGGCCATCTACAAG |
| | | TGTAGACCTGGCTACCGCAGCCTGG |
| | | GCAATGTGATCATGGTCTGCAGAAA |
| | | AGGCGAGTGGGTCGCCCTGAATCCT |
| | | CTGAGAAAGTGCCAGAAGAGGCCTT |
| | | GCGGACACCCCGGCGATACACCTTT |
| | | TGGCACATTCACCCTGACCGGCGGC |
| | | AATGTGTTTGAGTATGGCGTGAAGG |
| | | CCGTGTACACCTGTAACGAGGGATA |
| | | TCAGCTGCTGGGCGAGATCAACTAC |
| | | AGAGAGTGTGATACCGACGGCTGGA |
| | | CCAACGACATCCCTATCTGCGAGGT |
| | | GGTCAAGTGCCTGCCTGTGACAGCC |
| | | CCTGAGAATGGCAAGATCGTGTCCA |
| | | GCGCCATGGAACCCGACAGAGAGTA |
| | | TCACTTTGGCCAGGCCGTCAGATTC |
| | | GTGTGCAACAGCGGCTATAAGATCG |
| | | AGGGCGACGAGGAAATGCACTGCAG |
| | | CGACGACGGCTTCTGGTCCAAAGAA |
| | | AAGCCCAAATGCGTGGAAATCAGCT |
| | | GCAAGAGCCCCGACGTGATCAACGG |
| | | CAGCCCTATCAGCCAGAAGATCATC |
| | | TACAAAGAGAACGAGCGGTTCCAGT |
| | | ATAAGTGCAACATGGGCTACGAGTA |
| | | CAGCGAGCGGGGAGATGCCGTGTGT |
| | | ACAGAATCTGGATGGCGGCCTCTGC |
| | | CTAGCTGCGAGGAAAAGTCT |
| Compound 10 | 116 | GAATGTCCTCCTTGTCCTGCTCCTC |
| | | CAGTGGCCGGACCTTCCGTGTTTCT |
| | | GTTCCCTCCAAAGCCTAAGGACACC |
| | | CTGATGATCAGCAGAACCCCTGAAG |
| | | TGACCTGCGTGGTGGTGGACGTTTC |
| | | CCAAGAGGATCCCGAGGTGCAGTTC |
| | | AATTGGTACGTGGACGGCGTGGAAG |
| | | TGCACAACGCCAAGACCAAGCCTAG |
| | | AGAGGAACAGTTCAACAGCACCTAC |
| | | AGAGTGGTGTCCGTGCTGACCGTGC |
| | | TGCACCAGGATTGGCTGAACGGCAA |
| | | AGAGTACAAGTGCAAGGTGTCCAAC |
| | | AAGGGCCTGCCTAGCAGCATCGAGA |
| | | AAACCATCAGCAAGGCCAAGGGCCA |
| | | GCCAAGAGAACCCCAGGTTTACACC |
| | | CTGCCTCCAAGCCAAGAGGAAATGA |
| | | CCAAGAACCAGGTGTCCCTGACCTG |
| | | CCTGGTCAAGGGCTTCTACCCTTCC |
| | | GATATCGCCGTGGAATGGGAGAGCA |
| | | ATGGCCAGCCTGAGAACAACTACAA |
| | | GACCACACCTCCTGTGCTGGACAGC |
| | | GACGGCAGCTTTTTTCTGTACTCCC |
| | | GCCTGACCGTGGACAAGAGCAGATG |
| | | GCAAGAGGGCAACGTGTTCAGCTGC |
| | | TCTGTGATGCACGAGGCCCTGCACA |
| | | ACCACTACACCCAGAAGTCTCTGAG |
| | | CCTGTCTCTCGGAAAAGGCGGAGGC |
| | | GGAGCTGGTGGTGGTGCTGGCGGCG |
| | | GAGGATCTAAAGAAGATTGCAACGA |
| | | GCTGCCTCCTCGGCGGAATACCGAG |
| | | ATTCTGACAGGCTCTTGGAGCGACC |
| | | AGACATACCCTGAGGGCACCCAGGC |
| | | CATCTACAAGTGTAGACCTGGCTAC |
| | | CGCAGCCTGGGCAATGTGATCATGG |
| | | TCTGCAGAAAAGGCGAGTGGGTCGC |
| | | CCTGAATCCTCTGAGAAAGTGCCAG |
| | | AAGAGGCCTTGCGGACACCCCGGCG |
| | | ATACACCTTTTGGCACATTCACCCT |
| | | GACCGGCGGCAATGTGTTTGAGTAT |
| | | GGCGTGAAGGCCGTGTACACCTGTA |
| | | ACGAGGGATATCAGCTGCTGGGCGA |
| | | GATCAACTACAGAGAGTGTGATACC |
| | | GACGGCTGGACCAACGACATCCCTA |
| | | TCTGCGAGGTGGTCAAGTGCCTGCC |
| | | TGTGACAGCCCCTGAGAATGGCAAG |
| | | ATCGTGTCCAGCGCCATGGAACCCG |
| | | ACAGAGAGTATCACTTTGGCCAGGC |
| | | CGTCAGATTCGTGTGCAACAGCGGC |
| | | TATAAGATCGAGGGCGACGAGGAAA |
| | | TGCACTGCAGCGACGACGGCTTCTG |
| | | GTCCAAAGAAAAGCCCAAATGCGTG |
| | | GAAATCAGCTGCAAGAGCCCCGACG |
| | | TGATCAACGGCAGCCCTATCAGCCA |
| | | GAAGATCATCTACAAAGAGAACGAG |
| | | CGGTTCCAGTATAAGTGCAACATGG |
| | | GCTACGAGTACAGCGAGCGGGGAGA |
| | | TGCCGTGTGTACAGAATCTGGATGG |
| | | CGGCCTCTGCCTAGCTGCGAGGAAA |
| | | AGTCT |
| Compound 11 | 117 | GAATGTCCTCCTTGTCCTGCTCCTC |
| | | CAGTGGCCGGACCTTCCGTGTTTCT |
| | | GTTCCCTCCAAAGCCTAAGGACACC |
| | | CTGATGATCAGCAGAACCCCTGAAG |
| | | TGACCTGCGTGGTGGTGGACGTTTC |
| | | CCAAGAGGATCCCGAGGTGCAGTTC |
| | | AATTGGTACGTGGACGGCGTGGAAG |
| | | TGCACAACGCCAAGACCAAGCCTAG |
| | | AGAGGAACAGTTCAACAGCACCTAC |
| | | AGAGTGGTGTCCGTGCTGACCGTGC |
| | | TGCACCAGGATTGGCTGAACGGCAA |
| | | AGAGTACAAGTGCAAGGTGTCCAAC |
| | | AAGGGCCTGCCTAGCAGCATCGAGA |
| | | AAACCATCAGCAAGGCCAAGGGCCA |
| | | GCCAAGAGAACCCCAGGTTTACACC |
| | | CTGCCTCCAAGCCAAGAGGAAATGA |
| | | CCAAGAACCAGGTGTCCCTGACCTG |
| | | CCTGGTCAAGGGCTTCTACCCTTCC |
| | | GATATCGCCGTGGAATGGGAGAGCA |
| | | ATGGCCAGCCTGAGAACAACTACAA |
| | | GACCACACCTCCTGTGCTGGACAGC |
| | | GACGGCAGCTTTTTTCTGTACTCCC |
| | | GCCTGACCGTGGACAAGAGCAGATG |
| | | GCAAGAGGGCAACGTGTTCAGCTGC |
| | | TCTGTGATGCACGAGGCCCTGCACA |
| | | ACCACTACACCCAGAAGTCTCTGAG |
| | | CCTGTCTCTCGGAAAAGGCGGAGGC |
| | | GGAGCTGGTGGTGGTGCTGGCGGCG |
| | | GAGGATCTCGGGAAGATTGCAACGA |
| | | GCTGCCTCCTCGGCGGAATACCGAG |
| | | ATTCTGACAGGCTCTTGGAGCGACC |
| | | AGACATACCCTGAGGGCACCCAGGC |
| | | CATCTACAAGTGTAGACCTGGCTAC |
| | | CGCAGCCTGGGCAATGTGATCATGG |
| | | TCTGCAGAAAAGGCGAGTGGGTCGC |
| | | CCTGAATCCTCTGAGAAAGTGCCAG |
| | | AAGAGGCCTTGCGGACACCCCGGCG |
| | | ATACACCTTTTGGCACATTCACCCT |
| | | GACCGGCGGCAATGTGTTTGAGTAT |
| | | GGCGTGAAGGCCGTGTACACCTGTA |
| | | ACGAGGGATATCAGCTGCTGGGCGA |
| | | GATCAACTACAGAGAGTGTGATACC |
| | | GACGGCTGGACCAACGACATCCCTA |
| | | TCTGCGAGGTGGTCAAGTGCCTGCC |
| | | TGTGACAGCCCCTGAGAATGGCAAG |
| | | ATCGTGTCCAGCGCCATGGAACCCG |
| | | ACAGAGAGTATCACTTTGGCCAGGC |
| | | CGTCAGATTCGTGTGCAACAGCGGC |
| | | TATAAGATCGAGGGCGACGAGGAAA |
| | | TGCACTGCAGCGACGACGGCTTCTG |
| | | GTCCAAAGAAAAGCCCAAATGCGTG |
| | | GAAATCAGCTGCAAGAGCCCCGACG |
| | | TGATCAACGGCAGCCCTATCAGCCA |
| | | GAAGATCATCTACAAAGAGAACGAG |
| | | CGGTTCCAGTATAAGTGCAACATGG |
| | | GCTACGAGTACAGCGAGCGGGGAGA |
| | | TGCCGTGTGTACAGAATCTGGATGG |
| | | CGGCCTCTGCCTAGCTGCGAGGAAA |
| | | AGTCT |

### Assays for Fusion Protein Activity

### Hemolytic Assay

An exemplary complement pathway hemolysis assay measures complement-mediated lysis of rabbit erythrocytes secondary to activation of the alternative pathway on a cell surface. Rabbit erythrocytes generally activate complement-mediated lysis in mouse serum. As serum C3 is activated, C3 convertases, C3 activation fragments, and C5 convertases are deposited on rabbit RBCs. Serum alternative complement pathway activity in the presence of a fusion protein including factor H or a functional fragment of factor H and an Fc receptor binding domain (e.g., an IgG, or a functional fragment thereof, e.g., an Fc domain), for example, can be evaluated in a concentration-dependent manner. Incubation of rabbit erythrocytes in normal mouse serum is expected to cause cell lysis, while addition of nanomolar quantities of a fusion protein including factor H or a functional fragment of factor H and an Fc receptor binding domain, for example, is expected to decrease the degree of lysis.

### Complement Activity Assay

Alternative complement pathway activity can be evaluated in the fluid phase using an alternative complement pathway assay kit, for example, Complement system Alternative Pathway WIESLAB®, Lund, Sweden. This method combines principles of the hemolytic assay for complement activation with the use of labeled antibodies specific for a neoantigen produced as a result of complement activation. The amount of neoantigen generated is proportional to the functional activity of the alternative pathway. In the Complement system Alternative Pathway kit, wells of the plate are coated with specific activators of the alternative pathway. Serum is diluted in diluent containing specific blockers to ensure that only the alternative pathway is activated. During the incubation of the diluted patient serum in the wells, complement is activated by the specific coating. The wells are then washed and C5b-9 is detected with a specific alkaline phosphatase labeled antibody to the neoantigen as a result of complement activation. The amount of complement activation correlates with the color intensity and is measured in terms of absorbance (optical density (OD)). The addition of nanomolar quantities of a factor H fusion protein, for example, decreases the degree of activity.

Pharmaceutical Compositions. Dosage, and Administration

The fusion proteins described herein can be incorporated into pharmaceutical compositions suitable for administration to a subject. Pharmaceutical compositions including factor H fusion proteins described herein can be formulated for administration at individual doses ranging, e.g., from 0.01 mg/kg to 500 mg/kg. The pharmaceutical composition may contain, e.g., from 0.1 μg/0.5 mL to 1 g/5 mL of the fusion protein.

Compositions including factor H fusion proteins can also be formulated for either a single or multiple dosage regimens. Doses can be formulated for administration, e.g., hourly, bihourly, daily, bidaily, twice a week, three times a week, four times a week, five times a week, six times a week, weekly, biweekly, monthly, bimonthly, or yearly. Alternatively, doses can be formulated for administration, e.g., twice, three times, four times, five times, six times, seven times, eight times, nine times, ten times, eleven times, or twelve times per day.

The pharmaceutical compositions including factor H fusion proteins can be formulated according to standard methods. Pharmaceutical formulation is a well-established art (Gennaro (2000) Remington: The Science and Practice of Pharmacy, 20th Edition, Lippincott, Williams & Wilkins (ISBN: 0683306472); Ansel et al., (1999) Pharmaceutical Dosage Forms and Drug Delivery Systems. 7th Edition, Lippincott Williams & Wilkins Publishers (ISBN: 0683305727); and Kibbe (2000) Handbook of Pharmaceutical Excipients, American Pharmaceutical Association, 3rd Edition (ISBN: 091733096X).

The pharmaceutical composition can include the fusion protein and a pharmaceutically acceptable carrier. As used herein. “pharmaceutically acceptable carrier” includes any and all solvents, dispersion media, coatings, antibacterial and antifungal agents, isotonic and absorption delaying agents, and the like that are physiologically compatible. The term “pharmaceutically acceptable carrier” excludes tissue culture medium including bovine or horse serum. Pharmaceutically acceptable carriers or adjuvants, by themselves, do not induce the production of antibodies harmful to the individual receiving the composition nor do they elicit protection. Therefore, pharmaceutically acceptable carriers are inherently non-toxic and nontherapeutic, and are known to the person skilled in the art. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances include minor amounts of auxiliary substances such as wetting or emulsifying agents, preservatives, or buffers, which enhance the shelf life or effectiveness of the antibody.

The compositions described herein may be prepared in a variety of forms. These include, for example, liquid, semi-solid, and solid dosage forms, such as liquid solutions (e.g., injectable and infusible solutions), dispersions or suspensions, tablets, pills, powders, liposomes and suppositories. Such formulations can be prepared by methods known in the art (Eppstein et al., Proc. Natl. Acad. Sci. USA, 82:3688-92, 1985; Hwang et al., Proc. Natl. Acad. Sci. USA, 77:4030-4, 1980; and U.S. Pat. Nos. 4,485,045 and 4,544,545). Liposomes with enhanced circulation time are disclosed in, e.g., U.S. Pat. No. 5,013,556.

Pharmaceutical compositions including factor H fusion proteins can also be formulated with a carrier that protects the composition (e.g., a factor H fusion protein) against rapid release, such as a controlled-release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art (J. R. Robinson (1978) Sustained and Controlled Release Drug Delivery Systems, Marcel Dekker, Inc., New York).

The final form depends on the intended mode of administration and therapeutic application. Typical compositions are in the form of injectable or infusible solutions, such as compositions similar to those used for passive immunization of humans with other antibodies. The composition(s) can be delivered by, for example, parenteral injection (e.g., intravenous, subcutaneous, intraperitoneal, intramuscular).

The pharmaceutical compositions can be provided in a sterile form and stable under the conditions of manufacture and storage. The composition can be formulated as a solution, microemulsion, dispersion, liposome, or other ordered structure suitable to high drug concentration. Sterile injectable solutions can be prepared by incorporating the fusion protein in the required amount in an appropriate solvent with one or a combination of ingredients enumerated above, as required, followed by filter sterilization. Generally, dispersions are prepared by incorporating the fusion protein into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as, for example, lecithin, by the maintenance of the required particle size in the case of dispersion and by the use of surfactants. Prolonged absorption of injectable compositions can be brought about by including in the composition a reagent that delays absorption, for example, monostearate salts, and gelatin. The preferred form depends, in part, on the intended mode of administration and therapeutic application. For example, compositions intended for systemic or local delivery can be in the form of injectable or infusible solutions. The composition can be formulated, for example, as a buffered solution at a suitable concentration and suitable for storage at 2-8° C. (e.g., 4° C.). A composition can also be formulated for storage at a temperature below 0° C. (e.g., −20° C. or −80° C.). A composition can further be formulated for storage for up to 2 years (e.g., 1 month, 2 months, 3 months, 4 months, 5 months, 6 months, 7 months, 8 months, 9 months, 10 months, 11 months, 1 year, 1½ years, or 2 years) at 2-8° C. (e.g., 4° C.). Thus, the compositions described herein can be stable in storage for at least 1 year at 2-8° C. (e.g., 4° C.).

The fusion proteins described herein can be administered by a variety of methods known in the art, although for many therapeutic applications, the preferred route/mode of administration is intravenous injection or infusion. The fusion proteins can also be administered by intramuscular or subcutaneous injection. As will be appreciated by the skilled artisan, the route and/or mode of administration varies depending upon the desired results.

Biodegradable, biocompatible polymers can be used, such as ethylene vinyl acetate, polyanhydrides, polyglycolic acid, collagen, polyorthoesters, and polylactic acid. Prolonged absorption of injectable compositions can be attained by including in the composition an agent that delays absorption, for example, monostearate salts and gelatin. Many methods for the preparation of such formulations are known to those skilled in the art (see above). Additional methods applicable to the controlled or extended release of fusion proteins disclosed herein are described, for example, in WO 2016/081884, the entire contents of which are incorporated herein by reference.

The pharmaceutical composition(s) may have a pH of about 5.6-10.0, about 6.0-8.8, or about 6.5-8.0. For example, the pH may be about 6.2, 6.5, 6.75, 7.0, or 7.5. The pharmaceutical compositions can be formulated, for example, for oral, sublingual, intranasal, intraocular, rectal, transdermal, mucosal, topical, intravitreal, or parenteral administration. Parenteral administration may include intradermal, subcutaneous (s.c. s.q., sub-Q, Hypo), intramuscular (i.m.), intravenous (i.v.), intraperitoneal (i.p.), intra-arterial, intramedulary, intracardiac, intravitreal (eye), intra-articular (joint), intrasynovial (joint fluid area), intracranial, intraspinal, and intrathecal (spinal fluids) injection or infusion. Any device suitable for parenteral injection or infusion of drug formulations may be used for such administration. For example, the pharmaceutical composition may be contained in a sterile pre-filled syringe.

Additional active compounds can also be incorporated into the composition. In certain embodiments, a fusion protein is co-formulated with and/or co-administered with one or more additional therapeutic agents. When compositions are to be used in combination with a second active agent, the compositions can be co-formulated with the second agent, or the compositions can be formulated separately from the second agent formulation. For example, the respective pharmaceutical compositions can be mixed, e.g., just prior to administration, and administered together or can be administered separately, e.g., at the same or different times. In some embodiments, a fusion protein can be co-formulated and/or co-administered with one or more additional antibodies that bind other targets (e.g., antibodies that bind regulators of the alternative complement pathway). Such combination therapies may utilize lower dosages of the administered therapeutic agents, thus avoiding possible toxicities or complications associated with the various monotherapies. Additionally, the compositions described herein can be co-formulated or co-administered with other therapeutic agents to ameliorate side effects of administering the compositions described herein (e.g., therapeutic agents that minimize risk of infection in an immunocompromised environment, for example, anti-bacterial agents, anti-fungal agents and anti-viral agents).

Preparations of compositions containing factor H fusion proteins can be provided to a subject in combination with pharmaceutically acceptable sterile aqueous or non-aqueous solvents, suspensions, or emulsions. Examples of non-aqueous solvents are propylene glycol, polyethylene glycol, vegetable oil, fish oil and injectable organic esters. Aqueous carriers include water, water-alcohol solutions, emulsions, or suspensions, including saline and buffered medical parenteral vehicles including sodium chloride solution, Ringer's dextrose solution, dextrose plus sodium chloride solution, Ringer's solution containing lactose or fixed oils.

Intravenous vehicles can include fluid and nutrient replenishers, electrolyte replenishers, such as those based upon Ringer's dextrose, and the like. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, can be present in such vehicles. A thorough discussion of pharmaceutically acceptable carriers is available in Remington's Pharmaceutical Sciences (Mack Pub. Co., N.J. 1991).

The pharmaceutical compositions can include a “therapeutically effective amount” or a “prophylactically effective amount” of a fusion protein. A “therapeutically effective amount” refers to an amount effective, at dosages, and for periods of time necessary, to achieve the desired therapeutic result. A therapeutically effective amount of the antibody can vary according to factors such as the disease state, age, sex, and weight of the individual, and the ability of the fusion protein to elicit a desired response in the individual. A “prophylactically effective amount” refers to an amount effective, at dosages, and for periods of time necessary, to achieve the desired prophylactic result. In some embodiments, a prophylactic dose is used in subjects prior to or at an earlier stage of disease where the prophylactically effective amount will be less than the therapeutically effective amount.

Dosage regimens can be adjusted, for example, to provide the optimum desired response (e.g., a therapeutic or prophylactic response). For example, a single bolus may be administered, several divided doses may be administered over time, or the dose may be proportionally reduced or increased as indicated by the exigencies of the therapeutic situation. It is advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. It is to be noted that dosage values can vary with the type and severity of the condition to be alleviated. It is to be further understood that for any particular subject, specific dosage regimens should be adjusted over time according to the individual need and the professional judgment of the administering clinician.

The efficacy of treatment with a fusion protein as described can be assessed based on an improvement in one or more symptoms or indicators of the disease state or disorder being treated. An improvement of at least 10% (increase or decrease, depending upon the indicator being measured) in one or more clinical indicators is considered “effective treatment,” although greater improvements are preferred, such as 20%, 30%, 40%, 50%, 75%, 90%, or even 100%, or, depending upon the indicator being measured, more than 100% (e.g., two-fold, three-fold, ten-fold, etc., up to and including attainment of a disease-free state.

### Methods of Treatment Using the Fusion Proteins

The complement factor H fusion proteins described herein (e.g., Compound 1, Compound 2, Compound 3, Compound 4, Compound 5, Compound 7, Compound 8, Compound 9, Compound 10, and Compound 11, and variants thereof) can be used to treat diseases mediated by alternative complement pathway dysregulation by inhibiting the alternative complement pathway activation in a mammal (e.g., a human). The fusion protein(s) described herein can be used to treat a variety of alternative complement pathway-associated disorders. Such disorders include, without limitation, PNH, aHUS, IgA nephrology, lupus nephritis, C3G, dermatomyositis, systemic sclerosis, demyelinating polyneuropathy, pemphigus, membranous nephropathy, FSGS, bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, DDD, AMD, SLE, RA, MS, TBI, ischemia reperfusion injury, preeclampsia, or TTP

Desirably, the disorder to be treated is SLE, lupus nephritis, membranous nephropathy, IgA nephropathy, FSGS, pemphigus, bullous pemphigoid, epidermolysis bullosa acquisita, systemic sclerosis, ANCA vasculitis, hypocomplementemic urticarial vasculitis, immune complex small vessel vasculitis, PNH, AHUS, dermatomyositis, or autoimmune necrotizing myopathies.

A therapeutically effective amount of a complement factor H fusion protein, as disclosed herein, is administered to a mammalian subject, e.g., a human subject, in need of such treatment. The amount administered is sufficient to inhibit complement activation and/or restore normal alternative complement pathway regulation. The determination of a therapeutically effective dose is within the capability of practitioners in this art, however, as an example, in embodiments of the method described herein utilizing systemic administration of a fusion protein for the treatment diseases mediated by alternative complement pathway dysregulation, an effective human dose will be in the range of 0.01 mg/kg to 150 mg/kg (e.g., from 0.05 mg/kg to 500 mg/kg, from 0.1 mg/kg to 20 mg/kg, from 5 mg/kg to 500 mg/kg, from 0.1 mg/kg to 100 mg/kg, from 10 mg/kg to 100 mg/kg, from 0.1 mg/kg to 50 mg/kg, from 0.5 mg/kg to 25 mg/kg, from 1.0 mg/kg to 10 mg/kg, from 1.5 mg/kg to 5 mg/kg, or from 2.0 mg/kg to 3.0 mg/kg) or from 1 μg/kg to 1,000 μg/kg (e.g., from 5 μg/kg to 1,000 μg/kg, from 1 μg/kg to 750 μg/kg, from 5 μg/kg to 750 μg/kg, from 10 μg/kg to 750 μg/kg, from 1 μg/kg to 500 μg/kg, from 5 μg/kg to 500 μg/kg, from 10 μg/kg to 500 μg/kg, from 1 μg/kg to 100 μg/kg, from 5 μg/kg to 100 μg/kg, from 10 μg/kg to 100 μg/kg, from 1 μg/kg to 50 μg/kg, from 5 μg/kg to 50 μg/kg, or from 10 μg/kg to 50 μg/kg). The route of administration may affect the recommended dose. Repeated systemic doses are contemplated to maintain an effective level, e.g., to attenuate or inhibit complement activation in a subject's system, depending on the mode of administration adopted.

The methods proteins described herein are particularly useful for treating renal lesions characterized histologically by predominant C3 accumulation the glomerular basement membrane in the absence of significant deposition of immunoglobulin (Nester and Smith, Curr. Opin. Nephrol. Hypertens., 22:231-7, 2013) from aberrant regulation of the alternative pathway of complement, also known as C3 glomerulopathy (C3G). A subset of C3G is DDD, a rare kidney disease leading to persisting proteinuria, hematuria, and nephritic syndrome. Factor H deficiency and dysfunction in C3G, e.g., DDD, has been reported in several cases. For example, mutations in Factor H have been found in human subjects with DDD. Symptoms of DDD include, e.g., one or both of hematuria and proteinuria; acute nephritic syndrome; drusen development and/or visual impairment; acquired partial lipodystrophy and complications thereof; and the presence of serum C3 nephritic factor (C3NeF), an autoantibody directed against C3bBb, the C3 convertase of the alternative complement pathway (Appel et al., J. Am. Soc. Nephrol., 16:1392-403, 2005). Targeting Factor H to complement activation sites has therapeutic effects on an individual having DDD. In some embodiments, administering an effective dose to the individual a composition including a fusion molecule described herein is effective in treating DDD. The route of administration may affect the recommended dose. Repeated systemic doses are contemplated to maintain an effective level, e.g., to attenuate or inhibit complement activation in a subject's system, depending on the mode of administration adopted.

The compositions and methods described herein are particularly useful for treatment of renal inflammation caused by systemic lupus erythematosus (SLE), such as lupus nephritis. Lupus glomerulonephritis, includes diverse and complex morphological lesions, depending on the proportion of glomeruli affected by active or chronic lesions, the degree of interstitial inflammation or fibrosis, as well as vascular lesions (Weening et al., J. Am. Soc. Nephrol., 15:241-50, 2004). Lupus nephritis is a serious complication that occurs in a subpopulation of subjects with SLE. SLE is the prototypic autoimmune disease resulting in multi-organ involvement. This anti-self response is characterized by autoantibodies directed against a variety of nuclear and cytoplasmic cellular components. These autoantibodies bind to their respective antigens, forming immune complexes that circulate and eventually deposit in tissues. This immune complex deposition causes chronic inflammation and tissue damage. Complement pathways (including the alternative complement pathway) are implicated in the pathology of SLE, and thus fusion proteins provided herein are thus useful for treating lupus nephritis.

The methods described herein are particularly useful for treating macular degeneration, such as AMD. AMD refers to age-related deterioration or breakdown of the eye's macula, resulting in the loss of integrity of the cells and/or extracellular matrix of the normal macula and/or the loss of function of the cells of the macula. It is clinically characterized by progressive loss of central vision that occurs as a result of damage to the photoreceptor cells in an area of the retina called the macula. AMD encompasses all stages of AMD, including Category 2 (early stage), Category 3 (intermediate), and Category 4 (advanced) AMD. Also encompassed are the two clinical states for which AMD has been broadly classified: a wet form and a dry form, with the dry form making up to 80-90% of total cases. The proteins of the alternative complement pathway are central to the development of AMD (Zipfel et al., Adv. Exp. Med. Biol., 703:9-24, 2010). Analysis of ocular deposits in AMD subjects has shown a large number of inflammatory proteins including amyloid proteins, coagulation factors, and proteins of the complement pathway. A genetic variation in the complement Factor H substantially raises the risk of AMD, suggesting that uncontrolled complement activation underlies the pathogenesis of AMD (Edwards et al., Science, 308:421-4, 2005; Haines et al., Science, 308:419-21, 2005; Klein et al., Science, 308:385-9, 2005; Hageman et al., Proc. Natl. Acad. Sci. USA, 102:7227-32, 2005). In some embodiments, methods of treating AMD, include, but are not limited to, formation of ocular drusen, inflammation in the eye or eye tissue, loss of photoreceptor cells, loss of vision (including for example visual acuity and visual field), neovascularization (such as choroidal neovascularization or CNV), and retinal detachment. Other related aspects, such as photoreceptor degeneration, RPE degeneration, retinal degeneration, chorioretinal degeneration, cone degeneration, retinal dysfunction, retinal damage in response to light exposure (such as constant light exposure), damage of the Bruch's membrane, loss of RPE function, loss of integrity of the histoarchitecture of the cells and/or extracellular matrix of the normal macular, loss of function of the cells in the macula, photoreceptor dystrophy, mucopolysaccharidoses, rod-cone dystrophies, cone-rod dystrophies, anterior and posterior uveitis, and diabetic neuropathy, are also included.

The compositions and methods described herein are particularly useful for treatment of PNH. PNH is a consequence of clonal expansion of one or more hematopoietic stem cells with mutant PIG-A. The extent to which the PIG-A mutant clone expands varies widely among subjects. Another feature of PNH is its phenotypic mosaicism based on the PIG-A genotype that determines the degree of GPI-AP deficiency. For example, PNH III cells are completely deficient in GPI-APs, PNH II cells are partially (˜90%) deficient, and PNH I cells, which are progeny of residual normal stem cells, express GPI-AP at normal density. Classic PNH is characterized by a large population of GPI-AP deficient polymorphonuclear leukocytes (PMNs), cellular marrow with thyroid hyperplasia and normal or near-normal morphology and frequent or persistent florid macroscopic hemoglobinuria. PNH in the setting of another bone marrow failure is characterized by a relatively small percentage (<30%) of GPI-AP deficient PMNs, evidence of a concomitant bone marrow failure syndrome and intermittent or absent mild to moderate macroscopic hemoglobinuria. Subclinical or latent PNH is characterized by a small (<1%) population of GPI-AP deficient PMNs, evidence of a concomitant bone marrow failure syndrome and no clinical or biochemical evidence of intravascular hemolysis. Complement pathways (including the alternative complement pathway) are implicated in the pathology of PNH, and thus fusion proteins provided herein (e.g., Compound 1, Compound 2, Compound 3, Compound 4, Compound 5, Compound 7, Compound 8, Compound 9, Compound 10, and Compound 11, and variants thereof) are thus useful for treating PNH.

The compositions and methods described herein are particularly useful for treatment of aHUS, a rare disease characterized by low levels of circulating red blood cells due to their destruction (hemolytic anemia), low platelet count (thrombocytopenia) due to their consumption and inability of the kidneys to process waste products from the blood and excrete them into the urine (acute kidney failure), a condition known as uremia. Complement pathways (including the alternative complement pathway) are implicated in the pathology of aHUS, and thus fusion proteins provided herein (e.g., Compound 1, Compound 2, Compound 3, Compound 4, Compound 5, Compound 7, Compound 8, Compound 9, Compound 10, and Compound 11, and variants thereof) are thus useful for treating aHUS.

The compositions and methods described herein are particularly useful for treatment of dermatomyositis, a group of acquired muscle diseases called inflammatory myopathies that are characterized by chronic muscle inflammation accompanied by muscle weakness. The cardinal symptom is a skin rash that precedes or accompanies progressive muscle weakness. Dermatomyositis may occur at any age, but is most common in adults in their late 40s to early 60s, or children between 5 and 15 years of age. Complement pathways (including the alternative complement pathway) are implicated in the pathology of dermatomyositis, and thus fusion proteins provided herein (e.g., Compound 1, Compound 2, Compound 3, Compound 4, Compound 5, Compound 7, Compound 8, Compound 9, Compound 10, and Compound 11, and variants thereof) are thus useful for treating dermatomyositis.

The compositions and methods described herein are particularly useful for treatment of systemic scleroderma. Also called diffuse scleroderma or systemic sclerosis, it is a chronic disease characterized by diffuse fibrosis and vascular abnormalities in the skin, joints, and internal organs (especially the esophagus, lower GI tract, lungs, heart, and kidneys). Common symptoms include Raynaud phenomenon, polyarthralgia, dysphagia, heartburn, and swelling and eventually skin tightening and contractures of the fingers. Complement pathways (including the alternative complement pathway) are implicated in the pathology of systemic scleroderma, and thus fusion proteins provided herein (e.g., Compound 1, Compound 2, Compound 3, Compound 4, Compound 5, Compound 7, Compound 8, Compound 9, Compound 10, and Compound 11, and variants thereof) are thus useful for treating systemic scleroderma.

The compositions and methods described herein are particularly useful for treatment of demyelinating polyneuropathy, a neurological disorder characterized by progressive weakness and impaired sensory function in the legs and arms. The disorder, which is sometimes called chronic relapsing polyneuropathy, is caused by damage to the myelin sheath of the peripheral nerves. Complement pathways (including the alternative complement pathway) are implicated in the pathology of demyelinating polyneuropathy, and thus fusion proteins provided herein (e.g., Compound 1, Compound 2, Compound 3, Compound 4, Compound 5, Compound 7, Compound 8, Compound 9, Compound 10, and Compound 11, and variants thereof) are thus useful for treating demyelinating polyneuropathy.

The compositions and methods described herein are particularly useful for treatment of pemphigus, a group of rare autoimmune skin disorders that cause blisters and sores on the skin or mucous membranes, such as in the mouth or on the genitals. Complement pathways (including the alternative complement pathway) are implicated in the pathology of pemphigus, and thus fusion proteins provided herein (e.g., Compound 1, Compound 2, Compound 3, Compound 4, Compound 5, Compound 7, Compound 8, Compound 9, Compound 10, and Compound 11, and variants thereof) are thus useful for treating pemphigus.

The compositions and methods described herein are particularly useful for treatment of thrombotic thrombocytopenia purpura (TTP). TTP features numerous microscopic clots, or thomboses, in small blood vessels throughout the body. Red blood cells are subjected to shear stress that damages their membranes, leading to intravascular hemolysis. The resulting reduced blood flow and endothelial injury results in organ damage, including brain, heart, and kidneys. TTP is clinically characterized by thrombocytopenia, microangiopathic hemolytic anemia, neurological changes, renal failure, and fever. TTP is caused by autoimmune or hereditary dysfunctions that activate the coagulation cascade or the complement system (George, N. Engl. J. Med., 354:1927-35, 2006). TTP may arise from genetic or acquired inhibition of the enzyme ADAMTS13, a metalloprotease responsible for cleaving large multimers of von Willebrand factor (VWF) into smaller units, ADAMTS13 inhibition or deficiency ultimately results in increased coagulation (Tsai, J. Am. Soc. Nephrol., 14:1072-81, 2003). Patients suffering from TTP typically present in the emergency room with one or more of the following; purpura, renal failure, low platelets, anemia, and/or thrombosis, including stroke. Thrombocytopenia can be diagnosed by a medical professional as one or more of: (i) a platelet count that is less than 150,000/mm³ (e.g., less than 60,000/mm³); (ii) a reduction in platelet survival time, reflecting enhanced platelet disruption in the circulation; and (iii) giant platelets observed in a peripheral smear, which is consistent with secondary activation of thrombocytopoiesis. Because TTP is a disorder that is driven by hyperactivation of the alternative complement pathway, treatment with fusion proteins described herein to inhibit the alternative complement pathway activation may aid in stabilizing and/or correcting the disease.

The compositions and methods described herein are particularly useful for treatment of membranous nephropathy (MN) is a glomerular disease and the most common cause of idiopathic nephrotic syndrome in nondiabetic white adults. If untreated, about one-third of MN patients progress to end stage renal disease over 10 years. The incidence of ESRD due to MN in the United States is about 1.9/million per year. Most cases of PMN (70%) have circulating pathogenic IgG4 autoantibodies to the podocyte membrane antigen PLA2R. Complement components including C3, C4d, and C5b-9 are also commonly present, but not C1q, indicating that the lectin and potentially the alternative pathways of complement activation are involved Over time, IgG4 and C5b-9 deposition leads to podocyte injury, urine protein excretion and nephrotic syndrome (William G. Couser Primary Membranous Nephropathy Clin J Am Soc Nephrol 12: 983-997, 2017). Mice lacking factor B, an essential component of the alternative pathway of complement activation, did not exhibit C3 and C5b-9 deposition and did not develop albuminuna in a mouse model of MN (Wentian Luo, Florina Olaru, Jeffrey H. Miner. Laurence H. Beck, Jr, Johan van der Vlag. Joshua M. Thurman, and Dorin-Bogdan Borza Alternative Pathway Is Essential for Glomerular Complement Activation and Proteinuria in a Mouse Model of Membranous Nephropathy Front Immunol. 2018; 9: 1433). Therefore, complement inhibitors that reduce the amount of C3 and C5 convertases deposited in glomerular lesions may be effective treatments for this disease.

The compositions and methods described herein are particularly useful for treatment of focal segmental glomerulosclerosis is characterized by obliteration of glomerular capillary tufts with increased matrix deposition and scarring (D'Agati V D, Fogo A B, Bruijn J A, Jennette J C Pathologic classification of focal segmental glomerulosclerosis: a working proposal. Am J Kidney Dis. 2004 February; 43(2):368-82.). The incidence of FSGS has increased over the past decades and it is one of the leading causes of nephrotic syndrome in adults (Korbet S M Treatment of primary FSGS in adults. J Am Soc Nephrol. 2012 November; 23(11):1769-76). Spontaneous remission is rare (<5%) and presence of persistent nephrotic syndrome indicates a poor prognosis with 50% of patients progressing to end-stage renal disease (ESRD) 6-8 years after initial diagnosis (Korbet S M Clinical picture and outcome of primary focal segmental glomerulosclerosis Nephrol Dial Transplant. 1999; 14 Suppl 3:68-73). Primary FSGS is responsible for 3.3% of all the cases of end-stage renal disease (ESRD) resulting from primary kidney disease in the United States. The complement system has been shown to be activated in patients with primary FSGS and elevated levels of plasma Ba, indicative of activation of the alternative pathway, correlates with disease severity. Patients with low serum C3 had a significantly higher percentage of interstitial injury. Furthermore, renal survival was found to be significantly higher in patients with normal serum C3 as compared to those with low serum C3. Low serum C3 is indicative of complement activation. Therefore, activation of the complement system may play a crucial role in the pathogenesis and outcome of FSGS (Jian Liu, Jingyuan Xie, Xiaoyan Zhang, Jun Tong, Xu Hao, Hong Ren, Weiming, Wang, & Nan Chen. Serum C3 and Renal Outcome in Patients with Primary Focal Segmental Glomerulosclerosis. Scientific Reports, 2017, 7: 4095). In humans, tubulointerstitial deposition of the complement membrane attack complex (C5b-9) is correlated with interstitial myofibroblast accumulation and proteinuria. In the experimental focal segmental glomerulosclerosis, the intratubular formation of C5b-9 was found to promote peritubular myofibroblast accumulation. Myofibroblasts may act as sentinel inflammatory cells and deposit extracellular matrix. These cells may also constrict kidney tubules leading to atubular glomeruli. By this mechanism, complement activation may contribute to tubulointerstitial injury and fibrosis in FSGS. (Rangan G K, Pippin J W, Couser W G. C5b-9 regulates peritubular myofibroblast accumulation in experimental focal segmental glomerulosclerosis. Kidney Int. 2004; 66:1838-1848). Factor B and factor D-deficient mice have lower proteinuria than WT controls in the adriamycin-induced FSGS model, suggesting that activation of AP has a pathogenic role (Lenderink A M, Liegel K, Ljubanović D, Coleman K E, Gilkeson G S, Holers V M, Thurman J M. The alternative pathway of complement is activated in the glomeruli and tubulointerstitium of mice with adriamycin nephropathy. Am J Physiol Renal Physiol. 2007 August; 293(2):F555-64) (Turnberg D, Lewis M, Moss J, Xu Y. Botto M, Cook H T. Complement activation contributes to both glomerular and tubulointerstitial damage in adriamycin nephropathy in mice. J Immunol. 2006 Sep. 15; 177(6):4094-102. Furthermore, complement factor H deficient mice display higher C3b glomerular deposition and more severe kidney damage than wild-type controls. (Morigi M. Locatelli M. Rota C. Buelli S, Corna D. Rizzo P, Abbate M. Conti D, Perico L, Longaretti L, Benigni A, Zoja C, Remuzzi G A previously unrecognized role of C3a in proteinuric progressive nephropathy. Sci Rep. 2016 Jun. 27; 60:28445). Therefore, an inhibitor of the alternative pathway of complement activation may have clinical utility in FSGS.

### EXAMPLES

The following examples are put forth so as to provide those of ordinary skill in the art with a disclosure and description of how the methods and compounds claimed herein are performed, made. They are intended to be purely exemplary and are not intended to limit the scope of the disclosure.

### Example 1. Construction of the Factor H Fusion Proteins

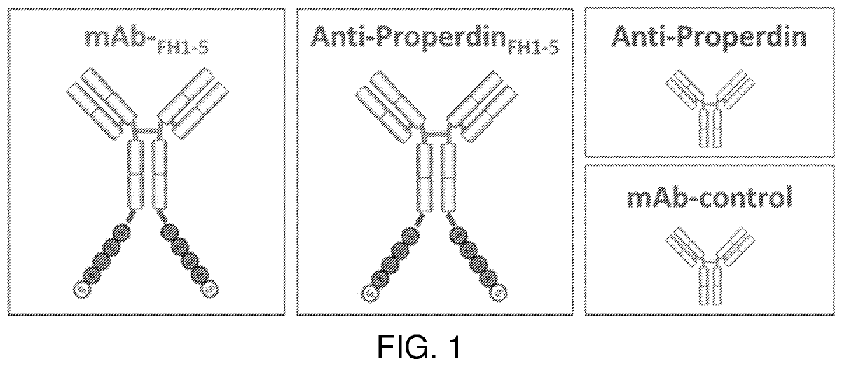

The fusion proteins, as shown in FIG. 1, include the first five SCRs of Factor H fused to the C-terminus of the heavy chain of an anti-properdin monoclonal IgG antibody or a non-targeting IgG monoclonal antibody. The proteins were transiently expressed in EXPI293 cells, followed by a single-step affinity purification process using protein-A resin. The anti-properdin antibody was utilized to target the fusion protein to the cell surface and engage with complement pathway components. The anti-properdin binding moiety regulates alternative complement activity at the cell surface. The anti-properdin binding moiety also regulates alternative complement AP activation mediated by properdin dependent nephritic factors. The N-terminus of SCRs 1-5 of factor H is attached to the C-terminus of the Fc. The fragment of Factor H regulates alternative complement activity in the fluid phase.

### Example 2. Mouse Models for Testing Factor H Fusion Proteins

To test the in vivo efficacy of the factor H fusion proteins described herein, Factor H knockout mice (Pickering et al., Nat. Genet., 31:424-8, 2002; Ruseva et al., J. Am. Soc. Nephrol., 24:43-52, 2013; Lesher et al., J. Am. Soc. Nephrol., 24:53-65, 2013; Ruseva et al., J. Am. Soc. Nephrol., 27:405-16, 2016) can be used to study the effect on the C3G/DDD-like phenotype of these mice. As a control, animals can be treated with IgG anti-properdin antibody without Factor H or the non-targeting monoclonal IgG antibody without factor H. For comparison, animals can be treated with a non-targeting monoclonal IgG antibody without Factor H; non-targeting monoclonal IgG antibodies with Factor H SCRs 1-5; an anti-properdin antibody without Factor H; or anti-properdin antibodies with Factor H SCRs 1-5. Non-targeting monoclonal IgG with factor H SCRs 1-5 and anti-properdin antibodies with Factor H SCRs 1-5 led to improvement (e.g., reduce C3 kidney deposits relative to control) of the disease.

Likewise, model of lupus nephritis (e.g., NZB/W F1 animal model, MRL/lpr animal model) are expected to yield similar results.

### Example 3. Effect of Factor H Fusion Proteins on Alternative Complement Pathway Activity

To examine the effect of the two factor H fusion proteins to restore complement regulatory activity in circulation and ameliorate glomerular complement deposition, factor H deficient mice, as described in Example 2, above, were injected intraperitoneally with a single dose of fusion proteins (non-targeting IgG-factor H SCRs 1-5 (mAb-FH₁₋₅), anti-properdin-factor H SCRs 1-5 (anti-P-FH₁₋₅)), or control antibodies (anti-properdin antibody without factor H or the non-targeting monoclonal IgG antibody; the non-targeting mAb control is the same throughout the examples and its Fc portion can bind Fc receptors). Blood samples were collected at 24 and 96 hours post administration to measure plasma factor B (FB), C3, C5 and properdin using ELISA and western blotting. Renal histology was examined to assess changes in glomerular C3 deposition by immunofluorescence.

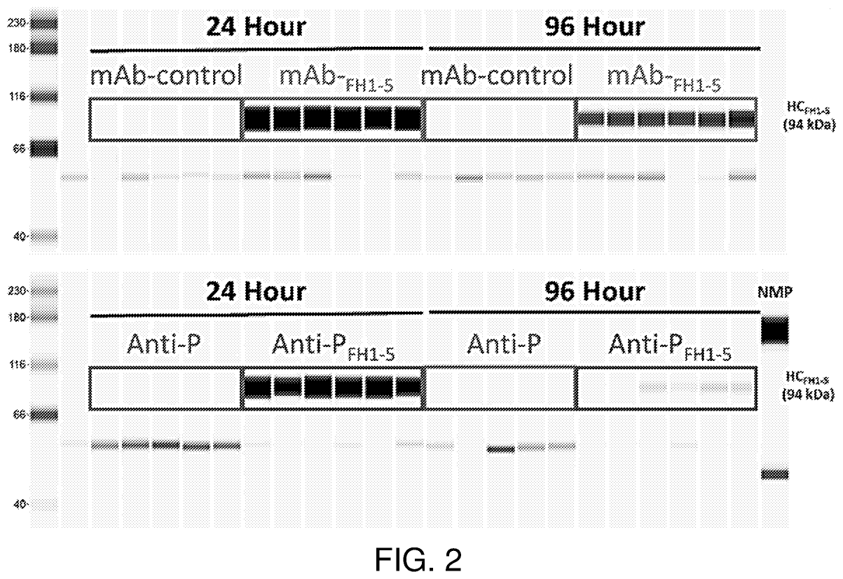

The IgG heavy chain component of both non-targeting mAb-FH₁₋₅ and anti-P-FH₁₋₅ was readily detectable in plasma at 24 hours; however, in contrast to anti-P-FH₁₋₅, non-targeting mAb-FH₁₋₅ was still detectable at 96 hours post administration, indicating a longer plasma half-life (FIG. 2). As the PK assay detects Fc-FH₁₋₅, control antibodies were not detected.

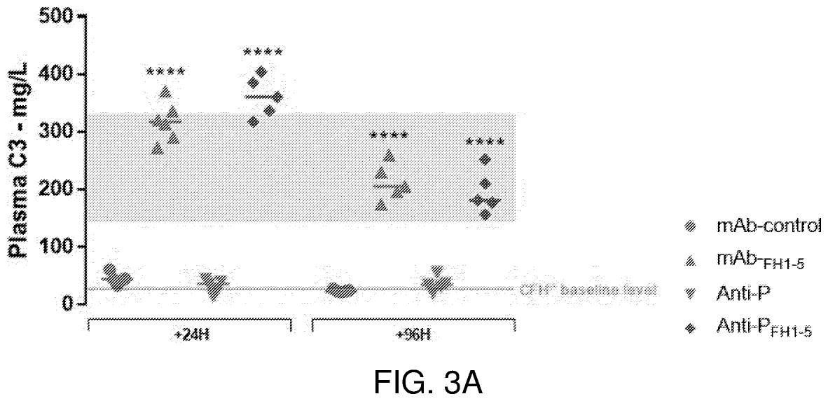

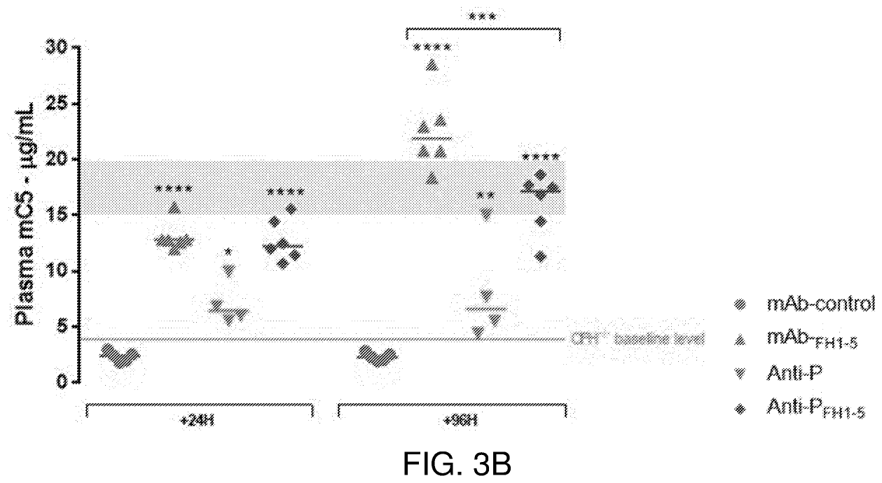

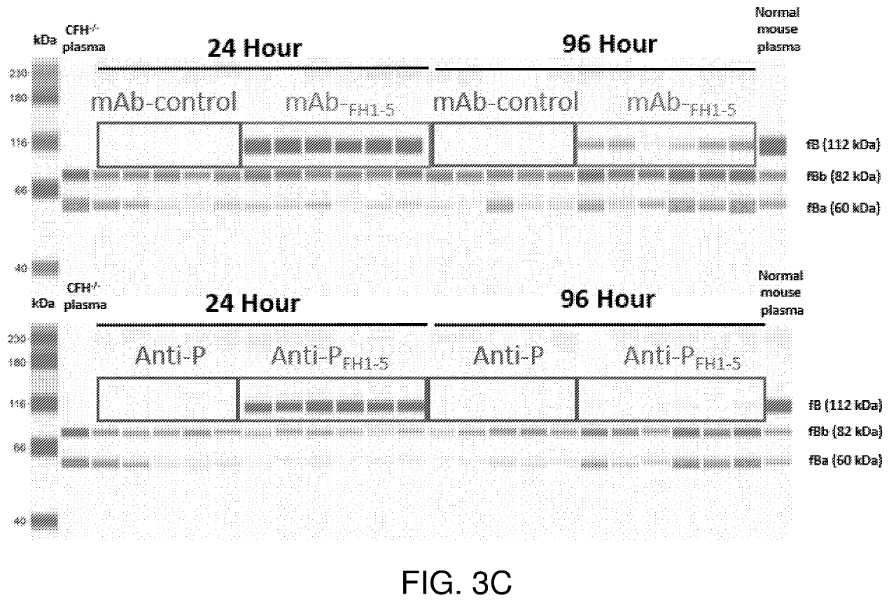

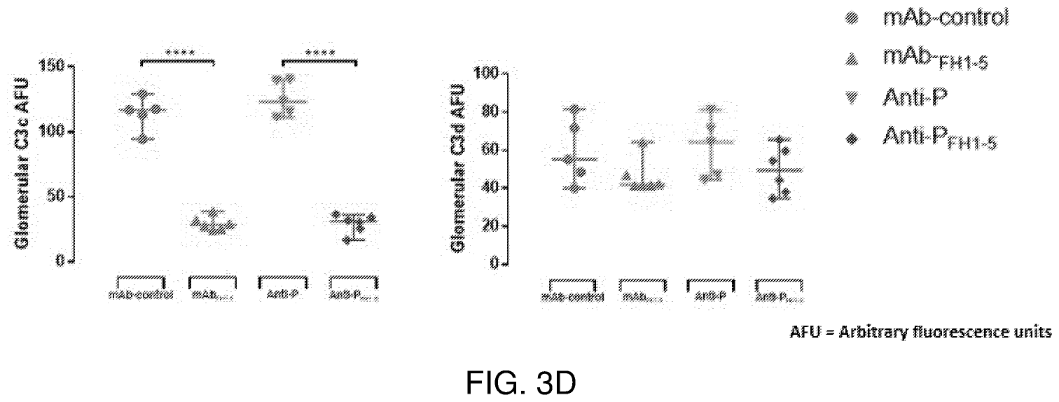

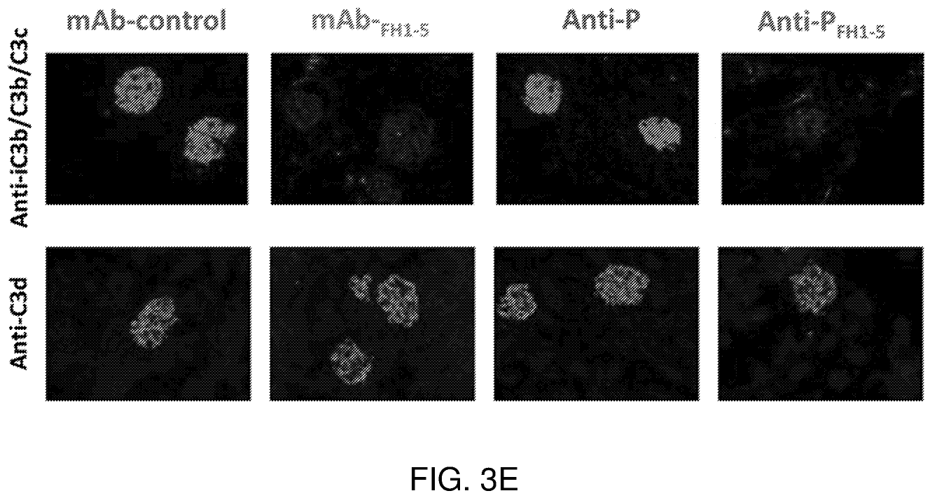

Following a single dose of either non-targeting mAb-FH₁₋₅ or anti-P-FH₁₋₅, plasma C3, FB, and C5 levels markedly increased and glomerular C3 deposition was reduced at 24 and 96 hours (FIGS. 3A-3C). The kinetics of these changes differed: factor B levels were lower at 96 hours compared to 24 hours, whereas plasma C3 and C5 levels remained within the normal range at both time points. Glomerular iC3b/C3b/C3c was reduced 96 hours after a single dose of either non-targeting mAb-FH₁₋₅ or anti-P-FH₁₋₅, while glomerular C3d was unaffected (FIGS. 3D and 3E).

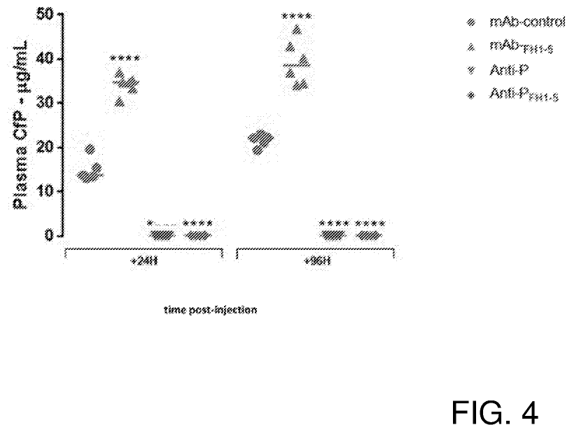

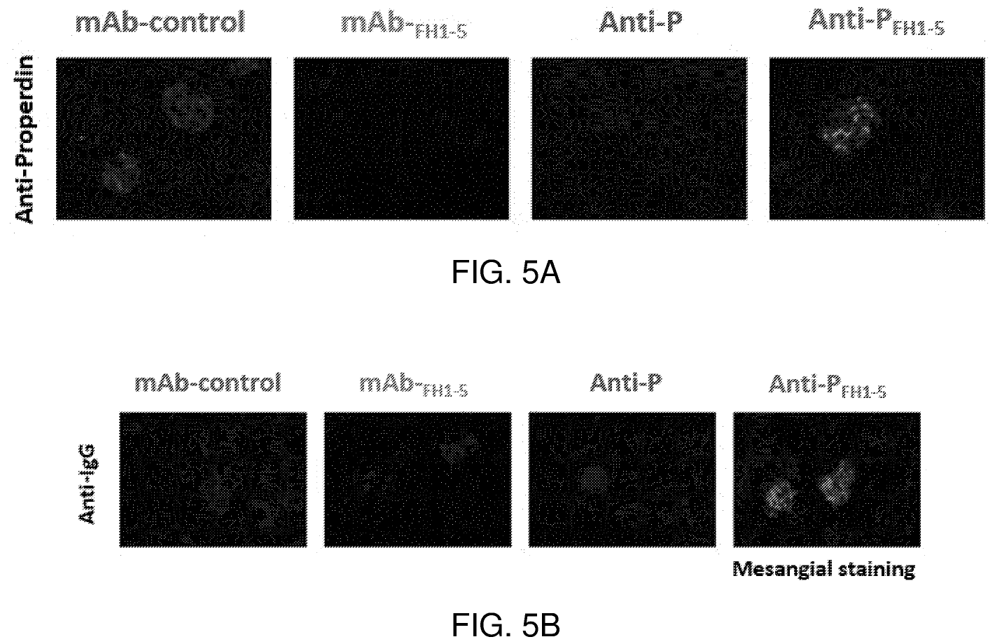

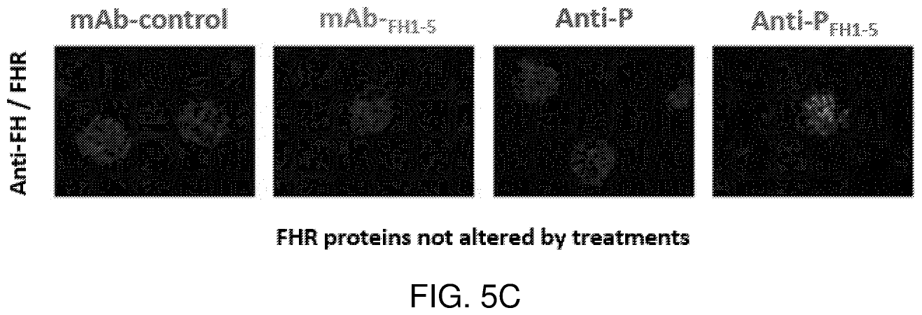

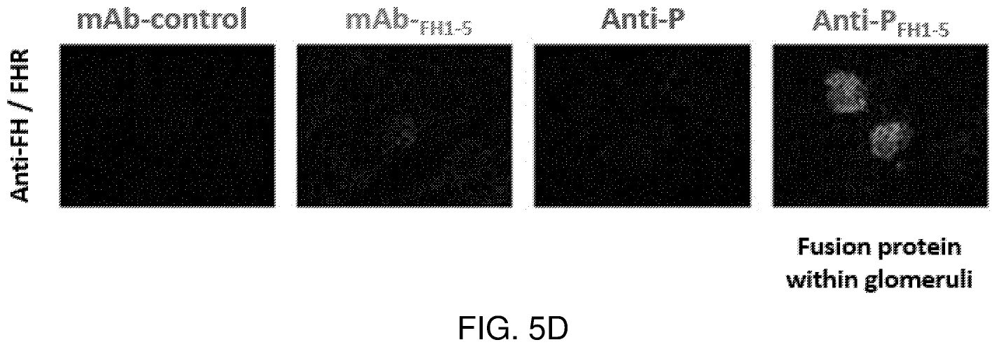

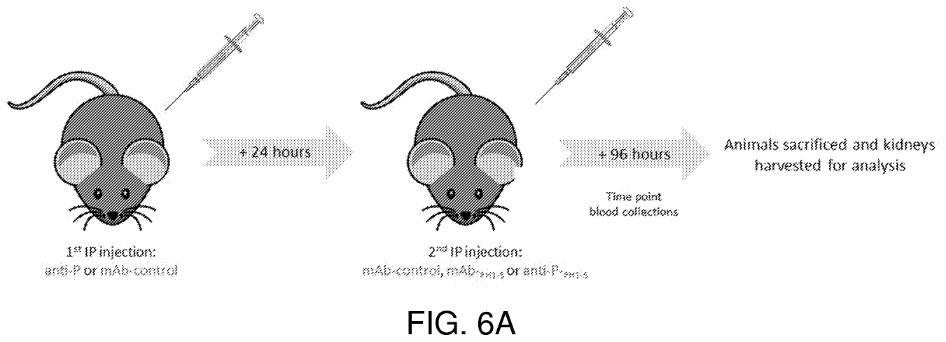

FH-deficient mice also have reduced plasma P levels and abnormal capillary wall P staining. Administration of anti-P-FH₁₋₅ resulted in depletion of plasma P, while non-targeting mAb-FH₁₋₅ increased circulating P levels to normal (FIG. 4). A single dose of non-targeting mAb-FH₁₋₅ or the anti-P control IgG abolished the abnormal glomerular properdin staining seen in FH-deficient mice, indicating that the turnover of glomerular properdin can be rapid (FIG. 5A).

A single dose of anti-P-FH₁₋₅ was associated with the appearance of mesangial reactivity to IgG, FH and properdin (FIGS. 5A-5C). This was consistent with the accumulation of this fusion protein in the mesangium, a finding confirmed in a FH/FH-related protein-deficient strain (FIG. 5D).

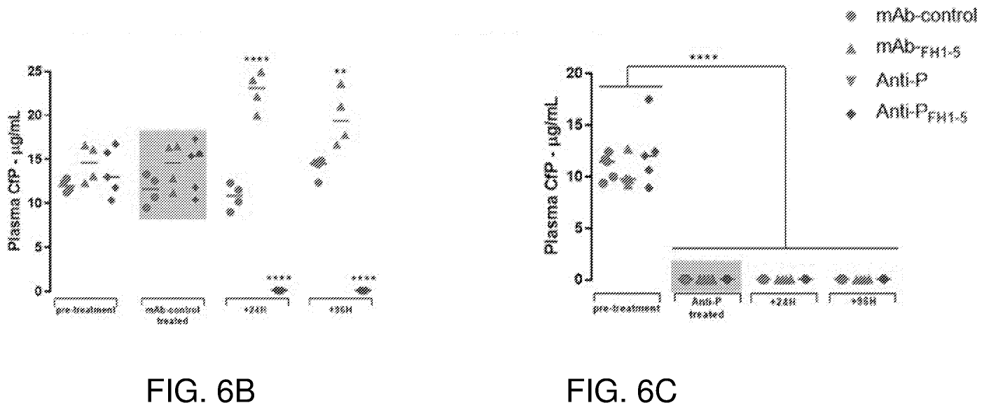

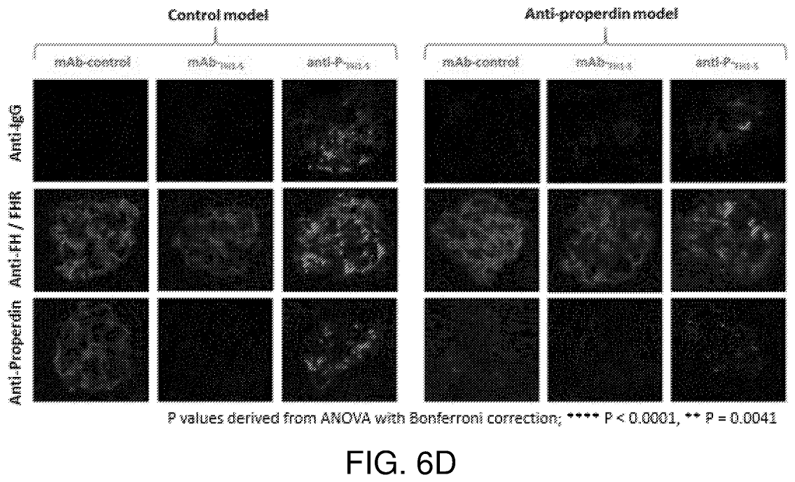

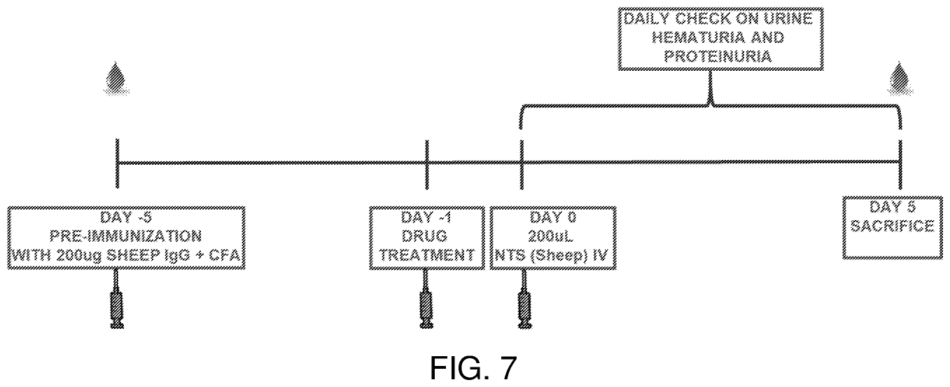

To further explore the mechanisms underlying the accumulation the fusion protein, FH^{-/-} mice were first depleted with an anti-P or mAb-control antibody and then injected with a non-targeting mAb control, non-targeting mAb-FH₁₋₅, or anti-P-FH₁₋₅. Blood was collected at 24 and 96 hours following the second dose. The animals were subsequently sacrificed, and kidneys were harvested for analysis (FIG. 6A). Though the deposition of anti-P-FH₁₋₅ was reduced, it was still observed in the properdin depleted CHF^{-/-} model, indicating that deposition of anti-P-FH₁₋₅ in the glomeruli is not fully reliant on properdin (FIGS. 6B, 6C, and 6D).

Overall, both non-targeting mAb-FH₁₋₅ and anti-P-FH₁₋₅ were effective at restoring plasma complement regulation and ameliorating the abnormal glomerular C3 deposition in FH-deficient mice.

Unexpectedly, the combination of targeting P in addition to FH-mediated regulation in the anti-P-FH₁₋₅ protein was associated with glomeruli deposition of the fusion protein without demonstrable improved or increased therapeutic efficacy over the non-targeting mAb-FH₁₋₅ protein. Non-targeting mAb-FH₁₋₅ fusion protein represents a potential therapeutic approach for C3G associated with FH deficiency. The data also indicate that glomerular P is rapidly cleared following restoration of alternative pathway regulation in FH deficiency.

### Example 4. Evaluation of the Effect of Fc-FH Fusion and mAb-FH Proteins in an In Vivo Model of Accelerated Nephrotoxin-Induced Nephritis (NTN)

The effect of Fc-FH fusion in attenuating NTN is evaluated using a knockout mouse model (FH/FHR DEL mouse). In CFH-deficient mice, spontaneous disease development takes 8 to 12 months. When nephrotoxin is injected in CFH-deficient mice, glomerular inflammation and proteinuria is induced, which resembles human type II membranoproliferative glomerulonephritis (MPGN). It was previously shown that protection against NTN in CFH-deficient mice can be achieved by treatment with anti-C5 (BB5.1) antibody therapy or C5-deficiency.

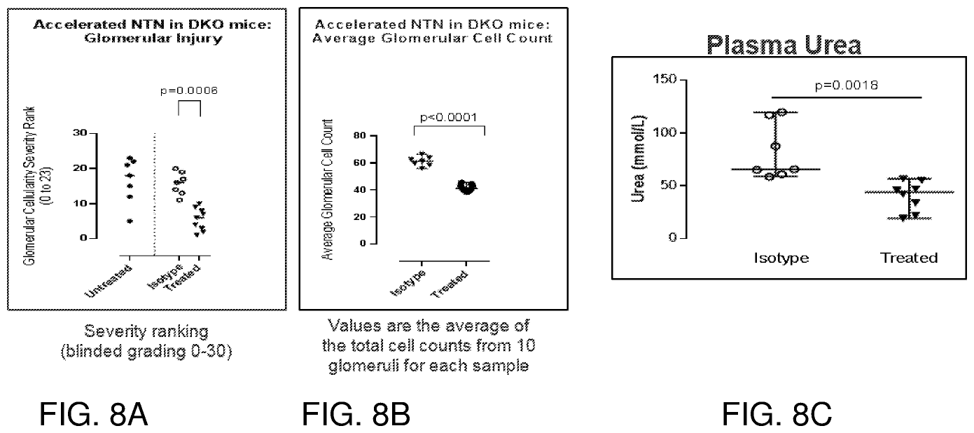

FIG. 7 provides a schematic outline of an example regimen for assaying for the effect of the molecules of the disclosure against accelerated NTN in an FH/FHR-deficient mouse model. Briefly, five days prior to the study, animals are to be divided into three cohorts, (a) non-treatment control group (N=10), (b) isotype-matched control antibody (in vivo mAb mouse IgG1 isotype control; MOPC1)-treated group (N=10), and (c) Fc-FH fusion-treated group (N=10) and pre-immunized with 200 μg sheep IgG in complete Freund's adjuvant (CFA). Four days after pre-immunization, the mice are to be treated with the control antibody (40 μg/Kg) or Fc-FH fusion (56 μg/Kg). Next day, the study is to be initiated by treating the animals with sheep nephrotoxic serum (NTS). Animals are to be sacrificed 5 days-post NTS injection.

The endpoints of the study include an assessment of kidney function by measuring plasma urea (e.g., by using an ultraviolet method) and an assessment of the severity of the glomerular injury and average infiltrated glomerular cell counts (inflammation).

Nephrotoxin injection in CFH-deficient mice markedly increases the plasma urea indicating the impaired kidney function. Treatment with an Fc-FH fusion protein significantly reduces plasma urea compared to mice from the control antibody-treated group. The improved kidney function with Fc-FH fusion protein treatment is accompanied by the intact kidney structure assessed with kidney sections stained with periodic acid-Shiff (PAS) reagent. Significant attenuation of glomerular injury and leukocyte infiltration are observed and quantified in Fc-FH fusion protein-treated animals compared to control-antibody treatment. Improvement in kidney pathology in Fc-FH fusion-treated animals is correlated with C3c and C9 (MAC) deposition which is notably reduced in the drug treatment group. Circulating levels of complement proteins C3 and C5 are restored with drug treatment and remain depleted in control groups.

In sum, the Fc-FH fusion protein confers in vivo therapeutic effects against nephrotoxin-induced membranoproliferative glomerulonephritis as it can attenuate kidney inflammation and injury; improve the renal function; clear C3c deposition in the kidney; attenuate C9 (MAC) deposition in the kidney; and normalize plasma C5 levels for a sustained duration.

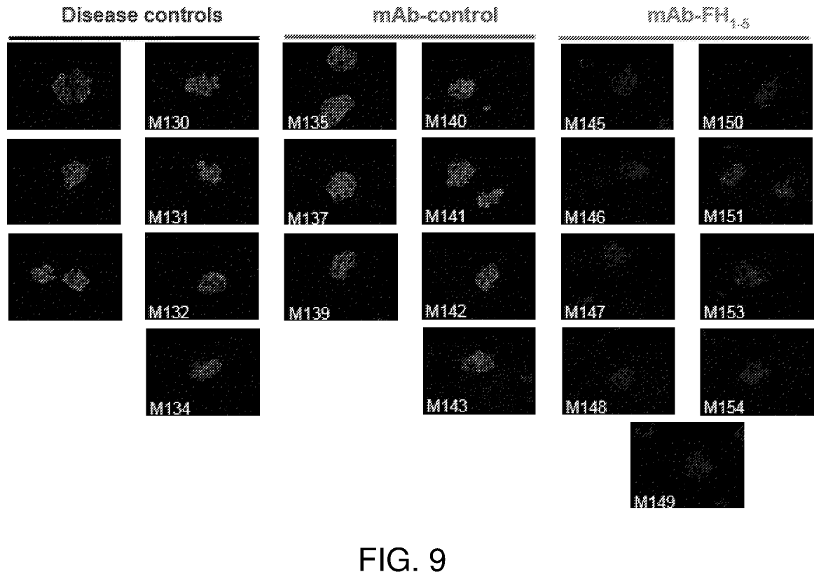

In a representative study, the effect of a fusion protein containing a non-targeting monoclonal antibody fused to a polypeptide including single chain repeats 1-5 (SCR1-5) of FH was evaluated (non-targeting mAb-FH₁₋₅). Results are shown in FIG. 8A, FIG. 8B, and FIG. 8C. It was found that non-targeting mAb-FH₁₋₅ treatment significantly attenuated glomerular injury and improved kidney function.

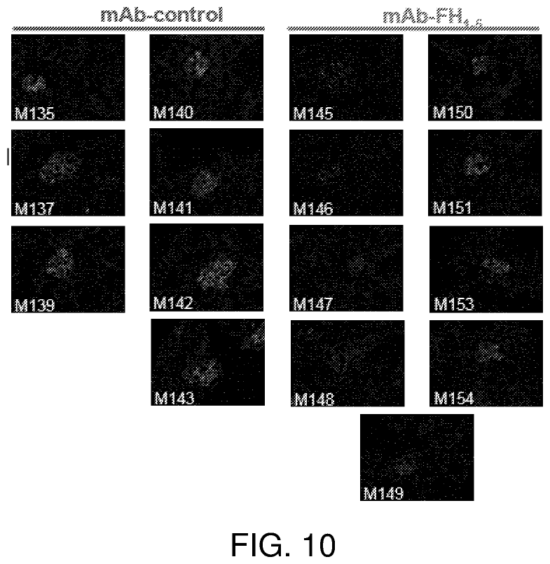

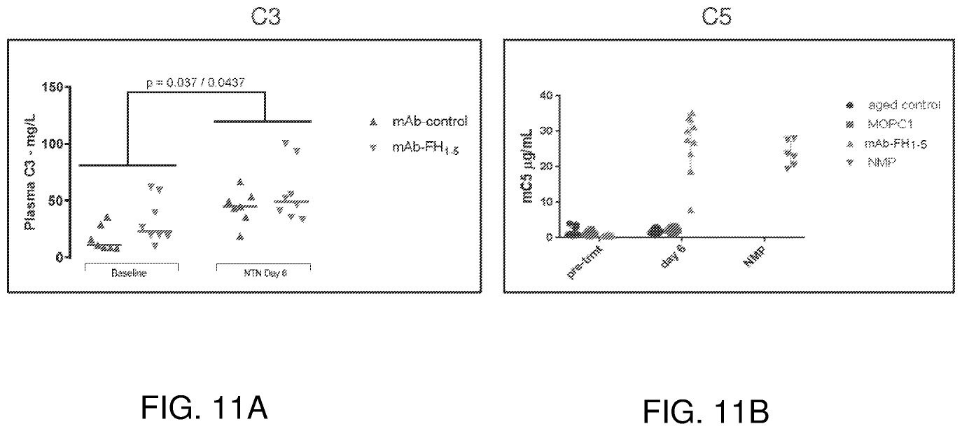

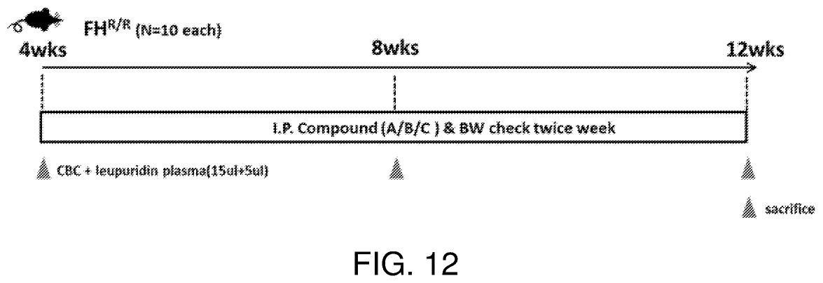

Further, non-targeting mAb-FH₁₋₅ treatment improved clearance of C3 in vivo (FIG. 9) and attenuated C9 (MAC) deposit in vivo (FIG. 10). Concomitantly, plasma C3 and plasma C5 levels were also significantly attenuated in mice treated with non-targeting mAb-FH₁₋₅ (FIG. 11A and FIG. 11B).

These results show that the non-targeting mAb-FH₁₋₅ fusion protein confers in vivo therapeutic effects against nephrotoxin-induced membranoproliferative glomerulonephritis as it can attenuate kidney inflammation and injury; improves the renal function; clears C3c deposition in the kidney; attenuates C9 (MAC) deposition in the kidney; and normalizes plasma C5 levels for a sustained duration.

### Example 5. Evaluation of the Effect of Fc-FH Fusion Proteins in an In Vivo Model of Atypical Hemolytic Uremic Syndrome (aHUS)

The effect of an Fc-FH fusion protein in attenuating aHUS is evaluated using a FH W1206R mutant mouse (FH^{R/R} mouse), which is recognized as a mouse model for human aHUS. The W1206R point mutation at SCR20 of factor H is directly derived from the well-documented human aHUS variant frequently found among aHUS patients. As this variant occurs at the C-terminal domain of factor H that functions to bind to the host surface, this variant does not affect the production or the regulatory function of factor H that is mediated by SCR1-SCR4 domains. Homozygous FH^{R/R} mice spontaneously develop aHUS symptoms, including low platelet counts, anemia, and thrombotic microangiopathy (TMA) which causes renal failure. Premature death has also been reported in FH^{R/R} mice. In addition to TMA in the kidney, FH^{R/R} also develop a systemic thrombotic disease with large thrombi formation in the liver or the spleen. The critical role of complement dysregulation in the model was previously shown in that complement deficiency (C3, factor D or properdin) or properdin blockade rescued the animals from the disease. Here, we compare the therapeutic the effect of Fc-FH fusion in comparison to properdin blockade.

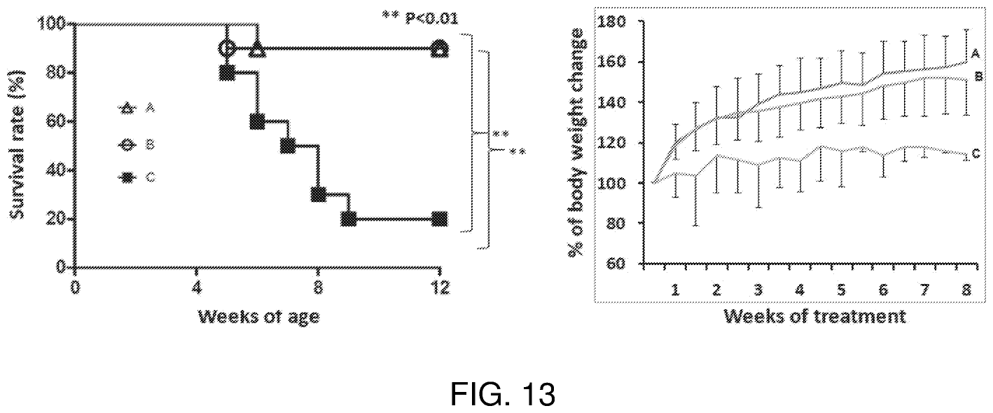

FIG. 12 provides a schematic outline of a regimen for assaying for the effect of the molecules of the disclosure against aHUS in FH^{R/R} mouse model. Briefly, animals are to be divided into three cohorts, (a) anti-properdin Mab treated control group (40 mg/Kg, N=10), (b) Fc-FH fusion-treated group (56 mg/Kg, N=10) (c) control antibody (in vivo MAb mouse IgG1 isotype control; MOPC1)-treated group (40 mg/Kg N=10). Throughout the period animals are to be treated with each molecule twice a week. Bloodwork (BW) of animals is checked twice a week. Complete blood count (CBC) is to be performed with lepuridin-treated plasma (15 μl and 5 μl) at regular intervals of 4 weeks (i.e., week 4, 8 and 12). Animals are to be sacrificed at 12 weeks.

The endpoints of the study are measurement of survival, body weight, blood test (platelet count, reticulocyte count, hemoglobin levels), including phenotypic assessment (e.g., measurement of necrotic tail; neurological abnormalities; liver hemorrhage; lung hemorrhage; splenomegaly; moribund; sudden death). Renal physiology is assessed using pathological assessment (periodic acid-Schiff (PAS) staining). Renal function is assessed by measuring a variety of parameters, such as proteinuria score, hematuria score, and blood urea nitrogen (BUN) levels. Further, histochemical assessment is carried out by sectioning kidneys post-sacrifice and staining for C3, fibrin, and further assessing large thrombi in kidney and liver tissue.

Control-antibody treated mice are characterized by increased mortality, with animals perishing well before the study endpoint. Kaplan-Meir survival curves show that mice treated with the Fc-FH fusion have significantly improved survival outcome compared to the animals in the control groups. Consistent with a previous report, mice treated with anti-properdin mAb also has improved survival outcome. With respect to the effect of drug therapy on body weight, it is seen that animals in the drug-treatment and the anti-properdin mAb treatment cohorts gain weight over an eight-week study period; whilst animals in the control-antibody treatment cohort do not gain weight during this time frame.

Improvement in thrombocytopenia and anemia in FH^{R/R} mice is evident in the Fc-FH fusion protein- or anti-properdin mAb-treated animals as platelet and hemoglobin counts are elevated at week 8 respectively, which are sustained until week 12 in both treatment groups. No change over the course of the study is seen in the control-antibody treated control group. Improvement in anemia is also reflected in the level of immature reticulocytes in the circulation which was reduced to the normal level with Fc-FH fusion or anti-properdin treatment.

Additionally, extra-renal phenotypes of FH^{R/R} mice are attenuated in the experimental group. Particularly, control-antibody treated FH^{R/R} mice developed splenomegaly and were moribund, showed propensity to develop necrotic tail, as well as neurological abnormalities and liver hemorrhage. These extra-renal phenotypes are significantly reduced by treating mice with the Fc-FH fusion protein.

In the assessment of renal pathology using periodic acid-Schiff (PAS) staining, kidneys of mice treated with the Fc-FH fusion protein or anti-properdin mAb maintained near normal glomerular structure without mesangial expansion and endothelial swelling with narrowing capillary lumen that are prominent in control mAb-treated animals. The PAS score correlates favorably with biomarker assessment of renal function, as evidenced by the fact that, compared to control-antibody treated control mice, proteinuria score, hematuria score, and blood urea nitrogen (BUN) levels are all attenuated in mice treated with the Fc-FH fusion protein or anti-properdin mAb.

In studies examining C3 and fibrin levels in kidney sections using immunostaining, treatment with Fc-FH fusion attenuates both C3 and fibrin deposits in the kidney of FH^{R/R} mice. While anti-properdin Mab treatment reduces fibrin levels but C3 levels appears to be increased which is consistent with the previous observation.

Liver and kidney tissue sections of FH^{R/R} mice show large thrombi, which can be identified via a standard haematoxylin and eosin stain (HE) staining method. In kidneys and livers of FH^{R/R} mice treated with Fc-FH fusion protein or anti-properdin Mab, incidence of large thrombi is virtually absent.

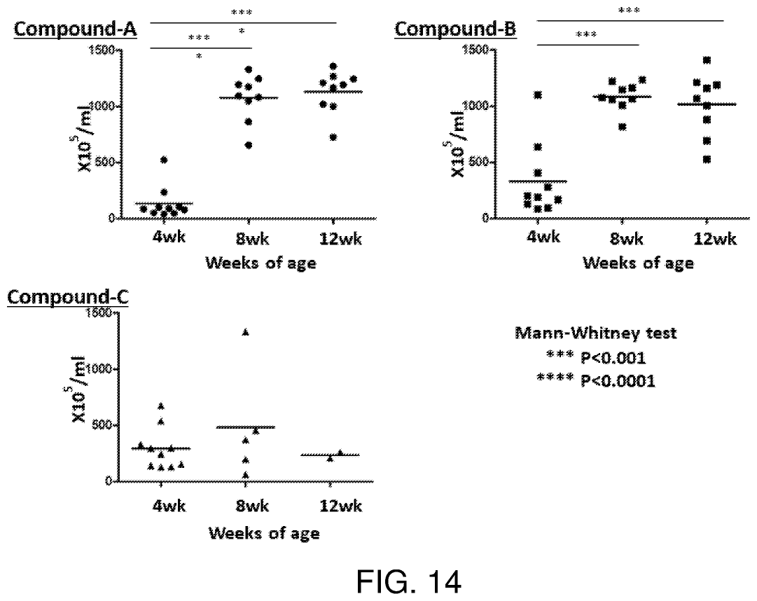

In a representative study, the in vivo effect of a fusion protein containing a non-targeting monoclonal antibody fused to a polypeptide including SCR1-5 of FH (non-targeting mAb-FH₁₋₅) on the survival and physiology of mice was evaluated. Here, treatment A includes therapy with the anti-properdin antibody alone; treatment B includes therapy with the non-targeting mAb-FH₁₋₅ molecule; and treatment C includes therapy with a non-targeting control antibody. Results are shown in FIG. 13. It was found that treatment with the non-targeting mAb-FH₁₋₅ fusion protein significantly increased survival and also increased weight of mice over an 8-week period compared to treatment with the control antibody.

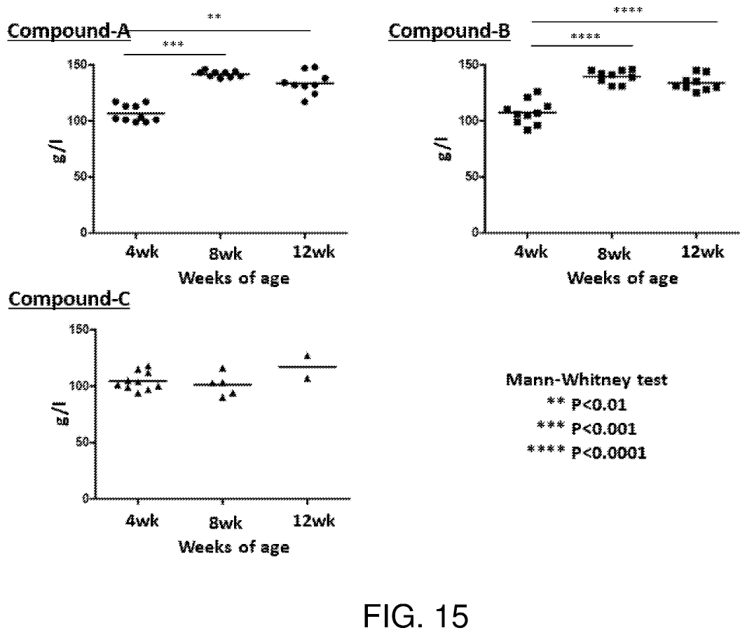

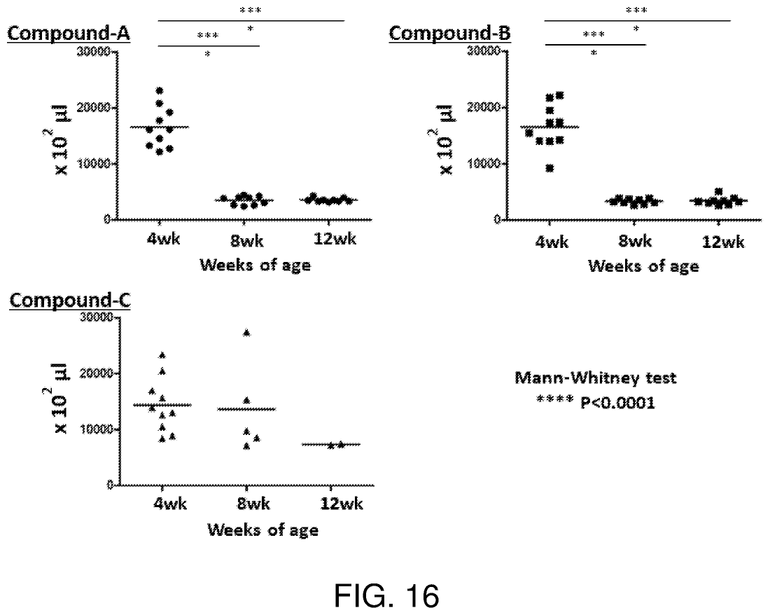

Thrombocytopenia and anemia were significantly reduced in mice treated with the non-targeting mAb-FH₁₋₅ fusion protein or anti-properdin mAb, as platelet and hemoglobin counts are elevated at week 8 respectively, which are sustained until week 12 in both treatment groups (FIG. 14 and FIG. 15, respectively). No change over the course of the study is seen in the control-antibody treated control group.

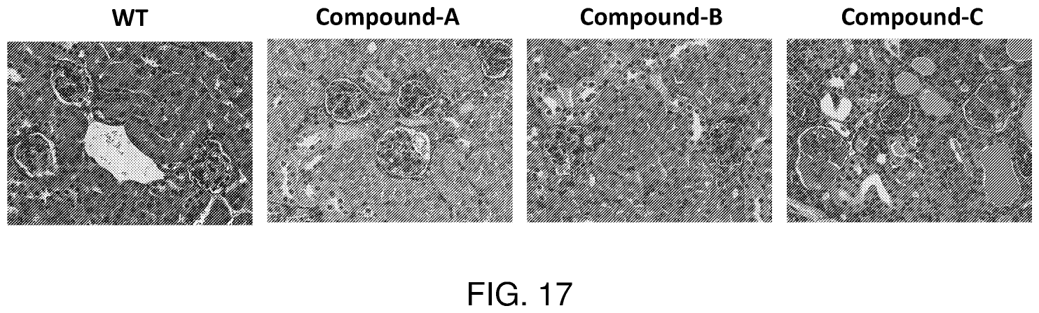

Reticulocyte levels are reduced in mice treated with the non-targeting mAb-FH₁₋₅ fusion protein or anti-properdin mAb (FIG. 16).

With respect to development of extra-renal phenotypes of FH^{R/R} mice, it was found that treatment with compounds reduced extra-renal phenotypes in vivo (Table 4). Particularly, control-antibody (compound C) treated FH^{R/R} mice developed splenomegaly and were moribund, showed propensity to develop necrotic tail, as well as neurological abnormalities and liver hemorrhage. These extra-renal phenotypes are significantly reduced by treating mice with the non-targeting mAb-FH₁₋₅ fusion (compound B) or anti-properdin Mab (compound A).

TABLE 4
Extra-renal phenotypes of FH^{R/R} mice
treated with various compounds.

| | Incidence (%) | | | | | |
|---|---|---|---|---|---|---|
| Phenotypes | Compound-A | | Compound-B | | Compound-C | |
| Necrotic tail | 0/10 | (0) | 0/10 | (0) | 4/10 | (40) |
| Neurological | 0/10 | (0) | 0/10 | (0) | 2/10 | (20) |
| abnormalities | | | | | | |
| Liver hemorrhage | 0/10 | (0) | 0/9 | (0) | 2/10 | (20) |
| Lung hemorrhage | 0/10 | (0) | 0/9 | (0) | 0/10 | (0) |
| Splenomegaly | 1/10 | (10) | 1/9 | (10) | 10/10 | (100) |
| Moribund | 1/10 | (10) | 0/10 | (0) | 8/10 | (80) |
| Sudden death | 0/10 | (0) | 1/10 | (10) | 0/10 | (0) |

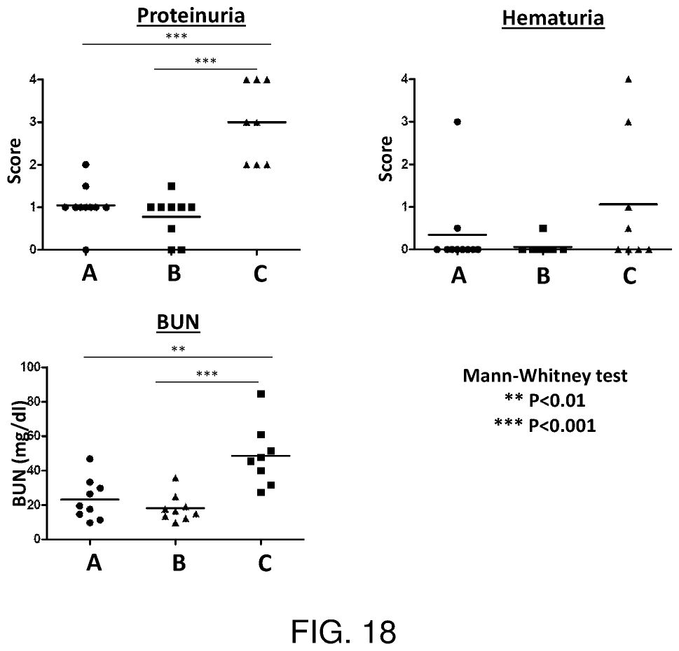

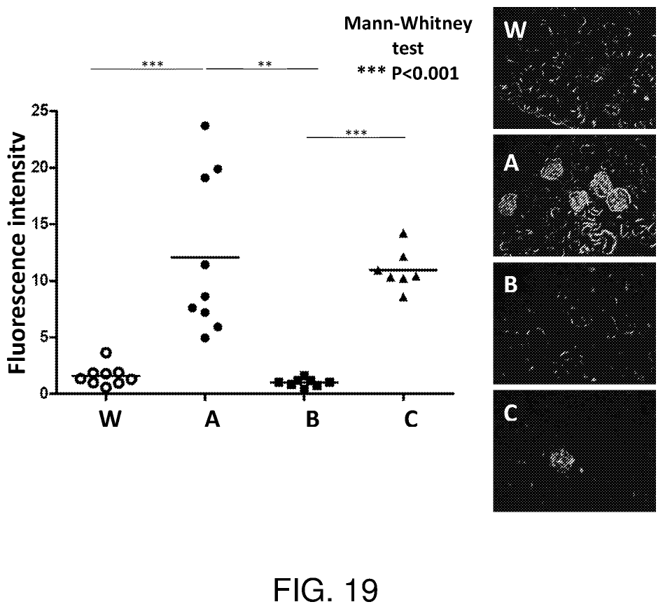

In the assessment of renal pathology using periodic acid-Schiff (PAS) staining, kidneys of mice treated with the non-targeting mAb-FH₁₋₅ fusion (compound B) or anti-properdin mAb (compound A) maintained near normal glomerular structure without mesangial expansion and endothelial swelling with narrowing capillary lumen that are prominent in non-targeting control mAb-(compound C) treated animals (FIG. 17). Further, treatment with non-targeting mAb-FH₁₋₅ (compound B) but not control antibody (compound C) reduces hematuria, proteinuria, and also blood urea nitrogen (BUN) in vivo (FIG. 18).

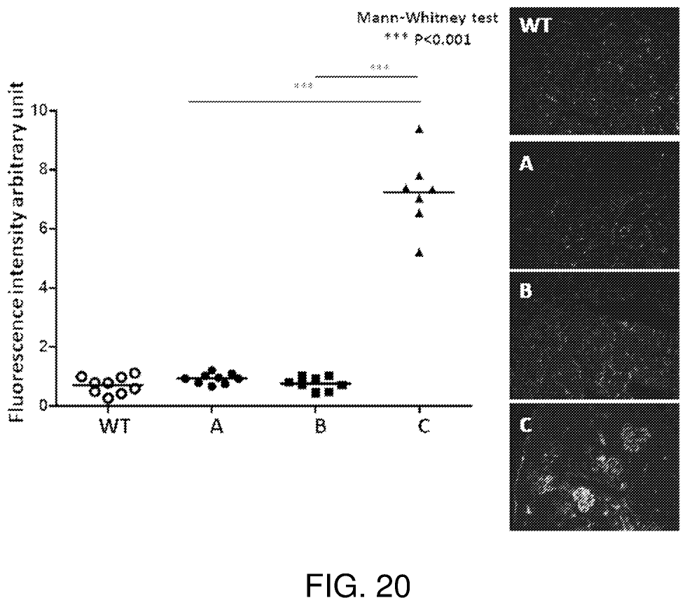

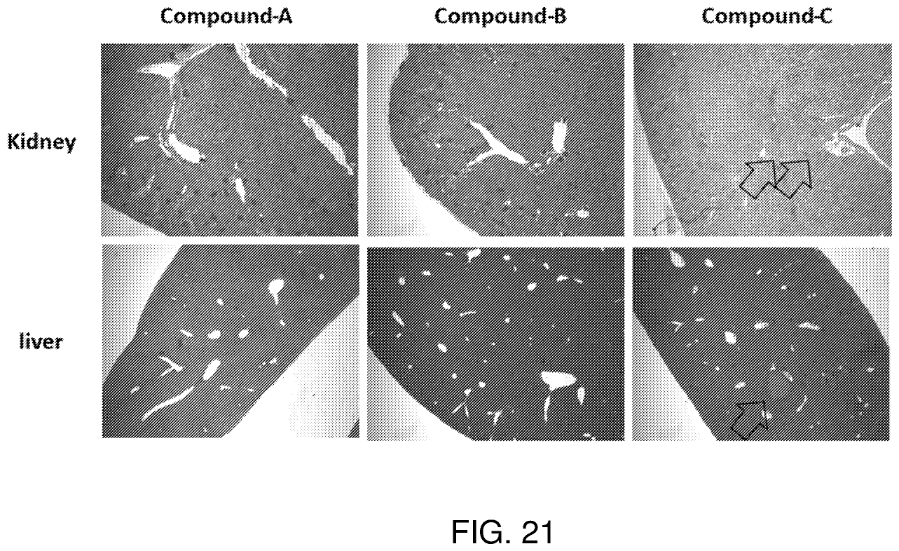

In studies examining C3 and fibrin levels in kidney sections using immunostaining, treatment with non-targeting mAb-FH₁₋₅ fusion attenuates both C3 and fibrin deposits in the kidney of FH^{R/R} mice, with levels similar to those of wild-type mice (FIG. 19 and FIG. 20). While anti-properdin Mab treatment reduces fibrin levels but C3 levels appear to be increased which is consistent with the previous observation (FIG. 19 and FIG. 20).

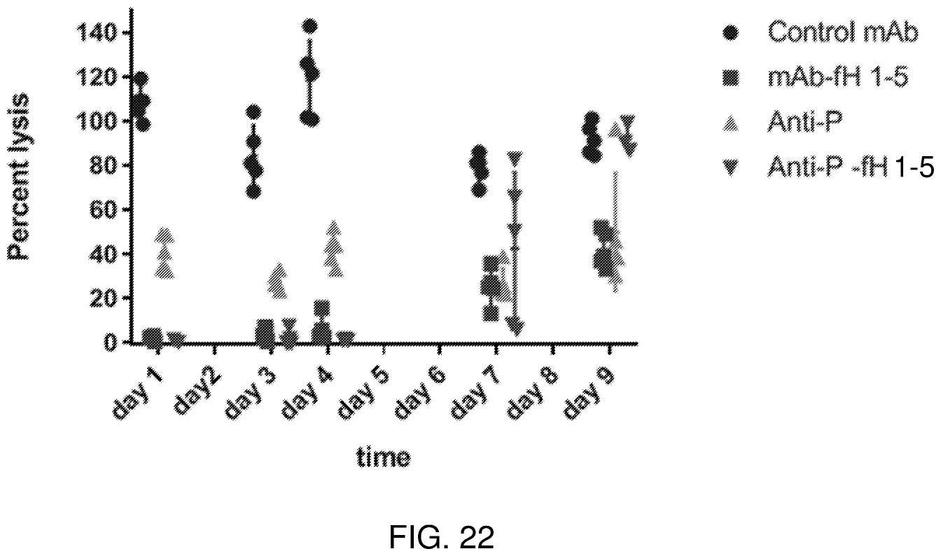

In evaluation of the effect of the non-targeting mAb-FH₁₋₅ fusion protein on large thrombi development in the kidneys and livers of FH^{R/R} mice, it was found that the incidence of large thrombi is virtually absent in mice treated with non-targeting mAb-FH₁₋₅ fusion protein, while 50% of control mAb-treated animal-derived kidneys and livers are affected (FIG. 21). Table 5 summarizes the findings.

TABLE 5
Effect of the treatment with various compounds in the incidence
of large thrombi in the kidneys or livers of FH^{R/R} mice.

| | Compound-A | | Compound-B | | Compound-C | |
|---|---|---|---|---|---|---|
| | incidence(%) | | | | | |
| Liver Thrombi | 0/10 | (0) | 0/9 | (0) | 5/10 | (50) |
| Kidney Thrombi | 0/10 | (0) | 0/9 | (0) | 5/10 | (50) |

### Example 6. Studies on Efficacy of the Molecules

### (1) Ex Vivo Assessment of Efficacy

The effectiveness of the FH fusion molecules was evaluated in vivo by dosing the fusion molecules (or antibodies with no FH fused) in wild type mice (C57BL/6). Control Mab or Anti-P (40 mg/Kg) or equivalent amount of the fusion molecules (56 mg/Kg) were dosed though the intraperitoneal route. Serum from treated animals was added diluted to 20% with alternative pathway specific assay buffer (GVBS buffer containing 10 mM EGTA, 10 mM MgCl₂).

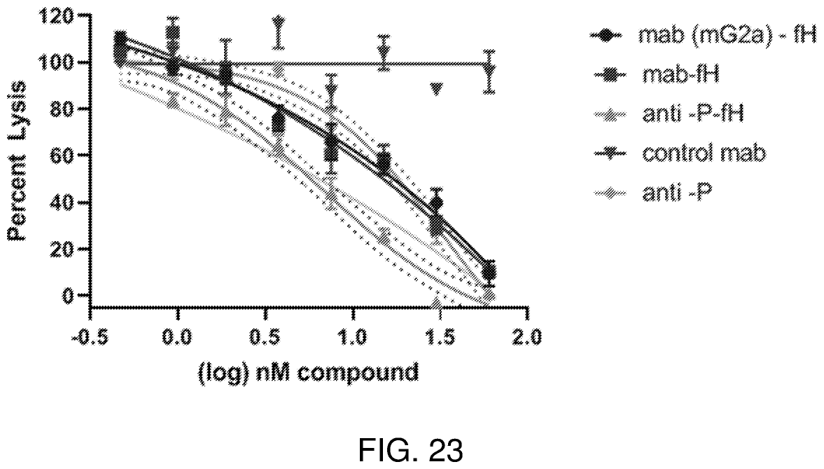

Washed rabbit erythrocytes were added at a final concentration of 1.5×10⁶ cells/mL and incubated with the diluted serum at 37° C. for 60 minutes. Plates were spun following incubation with erythrocytes and heme release quantified spectrophotometrically. FIG. 22 provides the relative hemolytic activity against the rabbit red blood cells over time in animals treated with each molecule. Near complete inhibition of alternative pathway hemolysis by fusion molecules lasted at least four days after dosing.

### (2) In Vitro Assessment of Efficacy of FH Fusion Molecules

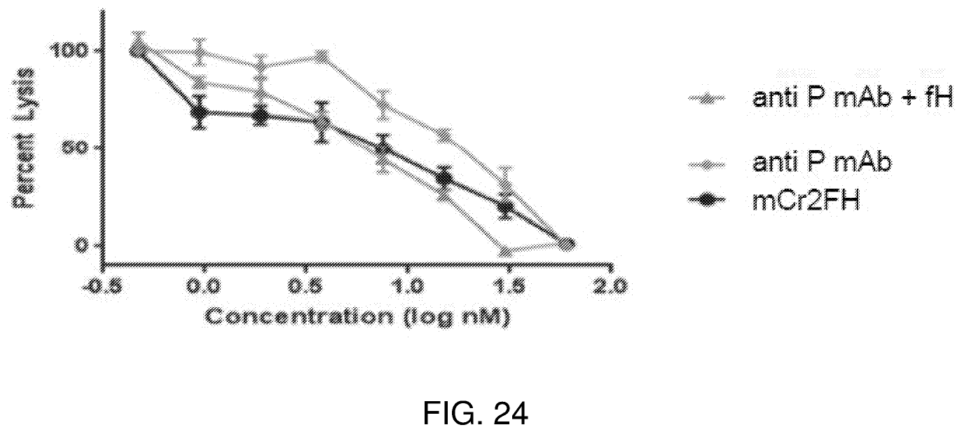

Antibodies were assessed functionally in vitro, using an alternative pathway (AP) specific hemolysis assay. Antibodies and normal mouse serum were diluted in alternative pathway specific lysis buffer (GVB⁰ supplemented with MgCl₂ and EGTA). Rabbit erythrocytes were washed and added to the antibody containing wells at a final concentration of 1.5×10⁶ cells/mL and incubated at 37° C. for 30 minutes. Plates were spun following incubation with erythrocytes and heme release quantified spectrophotometrically. FIG. 23 illustrates that all FH fusion antibodies inhibit alternative pathway hemolysis. Note that the control Mab without FH fusion, has no effect. Two Fc subclasses (mouse IgG1, and IgG2a) were assessed with FH fusion. Anti-Properdin antibody alone is an effective AP inhibitor.

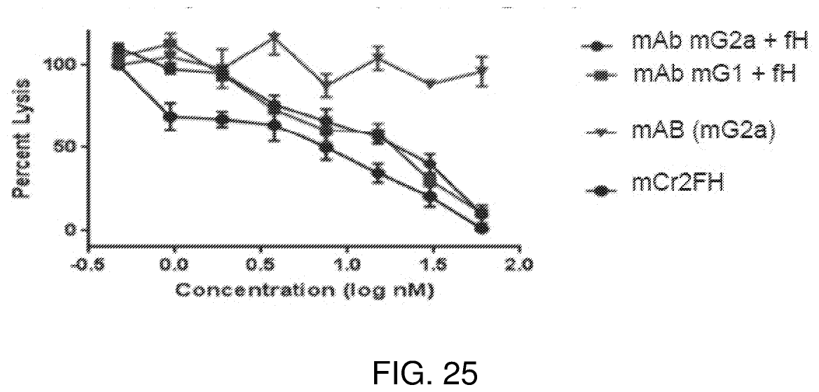

In a second study, the inhibitory activity of various fusion molecules was assessed using in vitro hemolysis assay, as outlined above. The following molecules were evaluated CR2 fused to FH; anti-properdin monoclonal antibody and anti-properdin mAb fused to FH. The inhibitory effect of anti-properdin mAb fused to FH was significantly greater than control anti-properdin mAb. The inhibitory effect of this anti-properdin mAb-FH fusion protein was comparable to or greater than CR2 fused to FH molecule. Data are shown in FIG. 24.

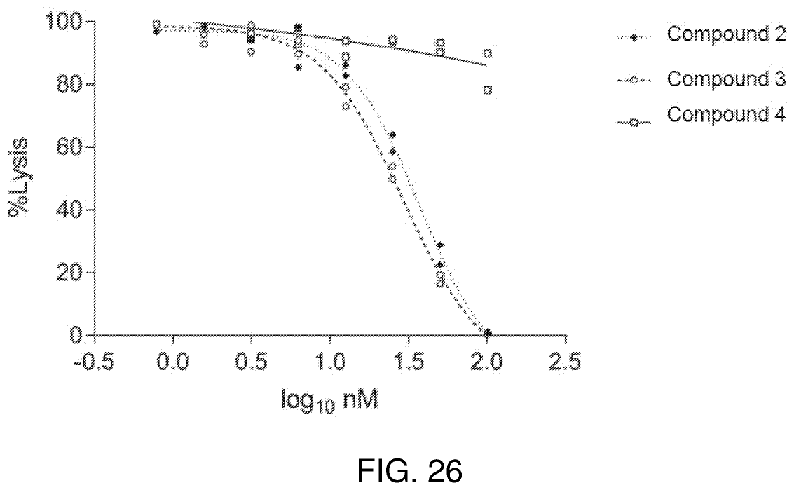

The second candidate, non-targeting mAb fused to FH₁₋₅ also showed stronger inhibitory effect than the control non-targeting mAb molecule. Data are shown in FIG. 25.

### Example 7. Analysis of In Vitro Potency

In this study, the in vitro potency of Compound 2, a molecule containing the hinge, CH2 and CH3 regions of a human IgG1 Fc region fused via a flexible linker to CFH SCRs 1-5 at the C-terminus, was analyzed. Compounds were tested for inhibition of the human complement alternative pathway in a rabbit red blood cell hemolysis assay. Here, rabbit red blood cells were incubated with titrations of both inhibitors for 30 minutes in 10% complement preserved human serum supplemented with 10 mM EGTA and 2 mM MgCl²⁺ in gelatin veronal buffer (GVB). These conditions allow for the activation of the complement alternative pathway but not the complement classical pathway. Red blood cell lysis was monitored by measuring the release of hemoglobin at 415 nM. In this experiment, Compound 2 was found to have an IC50 of 37 nM.

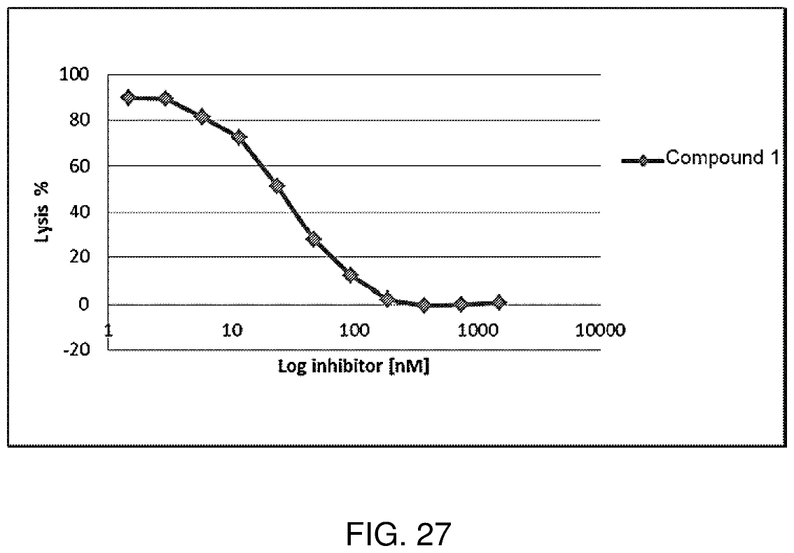

Next, compounds were tested for inhibition of the human complement alternative pathway in a rabbit red blood cell hemolysis assay. Here, rabbit red blood cells were incubated with titrations of both inhibitors for 30 minutes in 10% complement preserved human serum supplemented with 10 mM EGTA and 2 mM MgCl²⁺; buffer conditions in which the alternative pathway but not the classical pathway of complement may be activated. Red blood cell lysis was monitored by measuring the release of hemoglobin at 415 nM. The in vitro potency of factor H-Fc fusions without targeting domains was determined by testing serial dilutions of these compounds in the human alternative pathway complement hemolytic assay. FIG. 26 provides the dose-response curves for Compound 2, Compound 3, and Compound 4. As shown in the dose response curve, non-targeted compounds in which the CFH domain is attached to the C-terminus of the Fc region are active in this assay (Compound 2 and Compound 3) while Compound 4 having the CFH domain attached to the N-terminus of the Fc region was not active at the concentrations tested as shown in Table 6.

TABLE 6
Molecular descriptions and IC 50 values in the alternative complement
pathway hemolytic assay for non-targeted FH-Fc compounds

| | | IC50 |
|---|---|---|
| Compound | Description | (nM) |
| 2 | hIgG1Fc_(G4A)2G4S_fH SCR 1-5 | 37.0 |
| 3 | hIgG1 Fc_h fH SCRs 1-5 | 29.21 |
| 4 | hfH SCRs 1-5_(G4A)2G4S_IgG1Fc | nd |

### Example 8. Studies on In Vitro Activity and In Vivo Pharmacodynamics of the Molecules

DNA encoding Compound 1 (fusion protein comprising mouse IgG1 Fc fused via a flexible (G4A)2G3AG4S linker to mouse short consensus repeat domains (SCR) 1-4 of factor H) was prepared using standard recombinant technology. Human HEK293 cells were transiently transfected with a plasmid encoding Compound 1. After 4 days incubation the supernatant was collected and Compound 1 was purified by protein A chromatography. The in vitro potency of Compound 1 against the complement alternative pathway in mouse serum was assessed using the standard rabbit red blood cell hemolysis assay as described above.

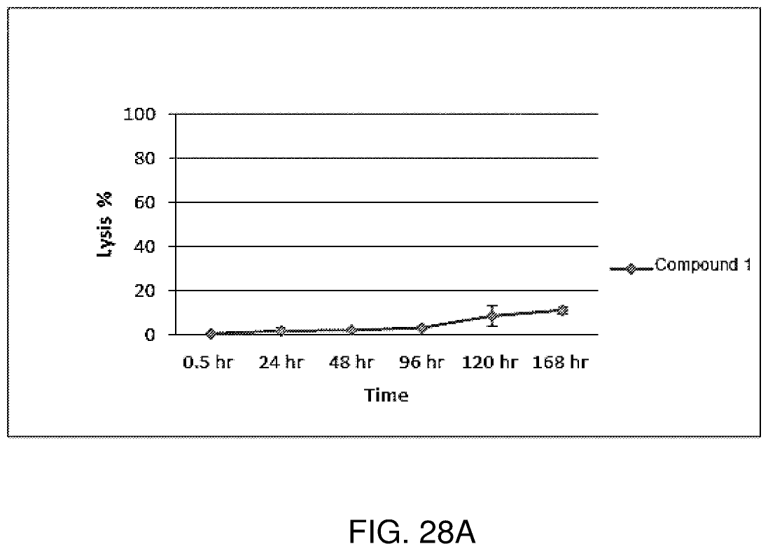

The results, which are presented in FIG. 27, show that Compound 1 was found to inhibit alternative complement pathway hemolysis in vitro with an IC50 value of 28.3 nM.

Next, ex vivo hemolytic assays were performed in 15.4% mouse serum. To determine the duration of inhibition of the alternative complement pathway in vivo, mice (C57BL/6; n=5) were administered a single, 25 mg/kg intravenous dose of the compound. Blood was collected at the indicated time-points and inhibition of the alternative complement pathway was monitored using the rabbit red blood cell alternative complement pathway hemolysis assay.

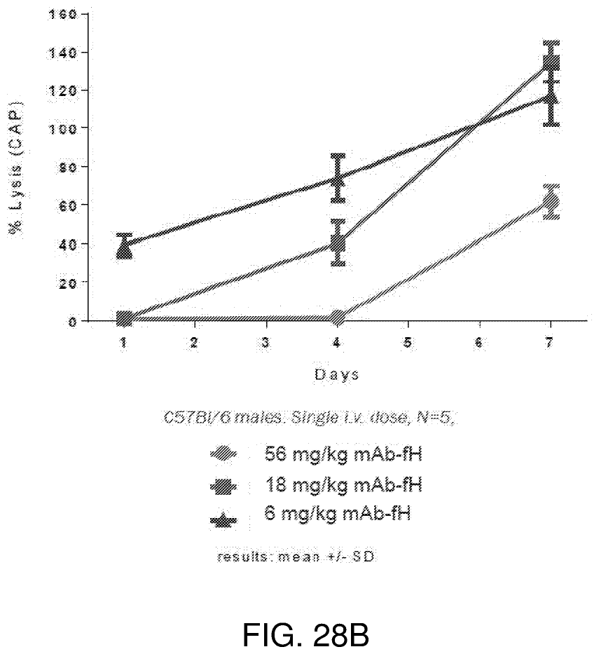

Results on the in vivo effect of the Fc-FH fusion protein against alternative complement pathway hemolysis are shown in FIG. 28A and FIG. 28B. FIG. 28A shows inhibitory activity of a single 25 mg/kg dose of Compound 1 (mouse IgG1 Fc fused to mouse short consensus repeat domains (SCR) 1-4 of factor H). Here AP hemolytic activity was substantially suppressed with only 11% hemolysis detected 168 hours after dosing. FIG. 28B shows in vivo inhibitory effect (mean±STD) of a single administration non-targeted mAb-FH₁₋₅ fusion proteins (triangle=6 mg/kg; square=18 mg/kg; and circle=56 mg/kg) in C57Bl/6 male mice.

### Example 9. Suppression of B-Cell Activation and Antibody Formation in the Mouse Keyhole Limpet Haemocyanin (KLH) Immunization Model

The pathology of certain diseases such as membranous nephropathy, IgA nephropathy, lupus, epidermolysis bullosa acquisita, dermatomyositis, and others involve the formation of autoantibodies that bind to self-structures, form immune complexes, and activate complement. The alternative complement pathway can further contribute to tissue damage by amplifying complement activation. Therefore, a therapeutic that can reduce alternative complement pathway activation and limit the complement-mediated stimulation of autoreactive B-cells may be effective in these diseases.

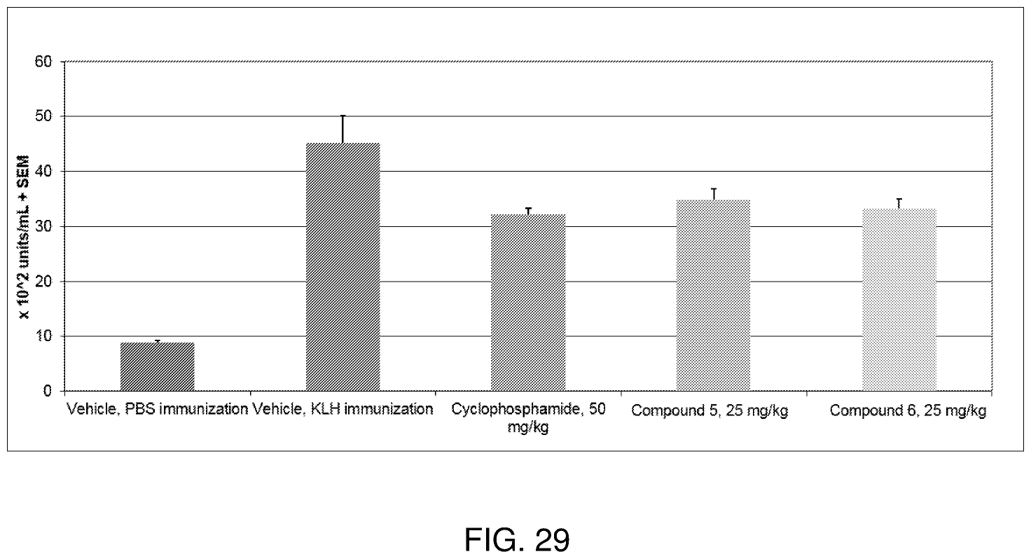

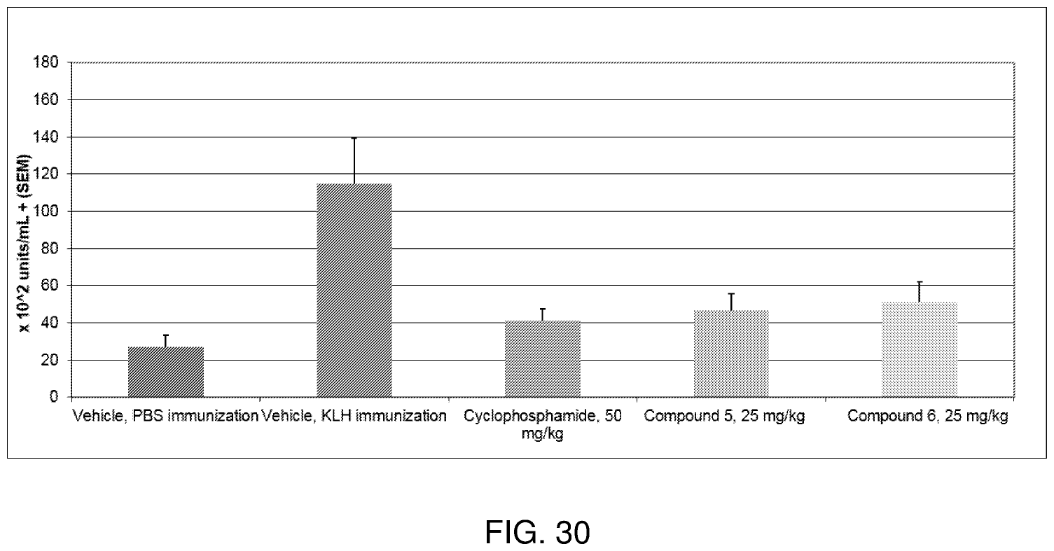

Compounds were evaluated for suppression of B-cell activation and antibody formation in the mouse KLH immunization model. Female C57BL/6 mice in groups of five were immunized with 0.5 mg KLH in 0.2 mL PBS by intraperitoneal injection (I.P.). On the day of immunization, mice were administered a single, 25 mg/kg I.P. dose of compounds Compound 5 and Compound 6 (CR2 SCRs 1-2 (N107Q)_(G4SDAA)_FLG2/G4 Fc_(G4A)2G3AG4S_fH SCR 1-4). As a positive control for inhibition of B-cell activation, one group of immunized mice received a 50 mg/kg dose of cyclophosphamide on the day of immunization and a second dose seven days later. Cyclophosphamide has been shown to reduce autoantibody formation in patients with lupus nephritis (Grootscholten et al., Arthritis Rheum. 56(3):924-937, 2007). One group of animals was immunized with KLH alone. As a negative control, one group of animals was sham-immunized with PBS. Serum samples were collected before immunization, 1 hour after immunization/dosing, on day 7 and on day 14. KLH specific IgM (early antibody response) and IgG (later response following class switching and affinity maturation) levels were determined by ELISA using KLH as the capture reagent. KLH immune serum from non-treated KLH immunized mice was used as a positive control in the ELISA. The statistical significance of antibody titers in treatment groups compared to the non-treated KLH immunized controls was determined using the Student's T-test. FIG. 29 provides the anti-KLH IgM data and FIG. 30 provides the anti-KLH IgG data. Statistically significant reductions in anti-KLH IgM titers compared to non-treated, immunized controls were observed for Compound 5 and Compound 6 and cyclophosphamide. The degree of suppression of the specific IgM response for these compounds was similar to that observed in the cyclophosphamide treated, immunized controls.

### Example 10. Treatment Diseases Associated with Alternative Complement Pathway Dysregulation in a Subject in Need Thereof

A subject can be diagnosed as having a disease associated with alternative complement pathway dysregulation (e.g., PNH, aHUS, IgA nephrology, lupus nephritis, C3G, dermatomyositis, systemic sclerosis, demyelinating polyneuropathy, pemphigus, membranous nephropathy, FSGS, bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, DDD, AMD, SLE, RA, MS, TBI, ischemia reperfusion injury, preeclampsia, or TTP) by a variety of diagnostic methods.

For example, a subject can be diagnosed as having DDD from electron microscopy analysis of biopsied tissue. A subject may exhibit plasma complement C3 lower than the normal range found in a healthy individual. The subject may exhibit nephrotic-range proteinuria, presented as elevated urinary protein excretion during a 24 hour time period. The subject may show elevated C3 nephritic factor, an autoantibody that stabilizes the alternative pathway C3 convertase activity. Genetic screening of the subject may reveal a tyrosine-402-histidine (Y402H) of Factor H, or other mutation in a regulator of the alternative complement pathway that is associated with DDD. A low level of plasmaC3 and C5, combined with a high level of C3 fragments and the terminal complement complex sC5b-9 and C5b-9 glomerular deposits can indicate abnormally high levels of alternative complement pathway activation.

In another example a subject may be diagnosed with C3 glomerulonephritis by a renal biopsy. The renal biopsy of a subject may demonstrate expansion of the mesangial matrix and increased glomerular cellularity, segmental capillary wall thickening and focal tubular atrophy. Electron microscopy may show sub-endothelial and mesangial electron dense deposits with infrequent sub-epithelial deposits. The biopsy may show positive staining for complement C3. The subject may exhibit proteinuria and renal impairment. The subject may have a family history of renal disease

A fusion protein including factor H or a functional fragment of factor H and an Fc receptor binding domain can be administered at an effective dose to treat the subject diagnosed with disease associated with alternative complement pathway dysregulation (e.g., PNH, aHUS, IgA nephrology, lupus nephritis, C3G, dermatomyositis, systemic sclerosis, demyelinating polyneuropathy, pemphigus, membranous nephropathy, FSGS, bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, DDD, AMD, SLE, RA, MS, TBI, ischemia reperfusion injury, preeclampsia, or TTP). In particular, a fusion protein containing the five N-terminal SCR domains of Factor H and an Fc receptor binding domain (e.g., an Fc domain) is administered to a subject. Alternatively, a fusion protein containing the four N-terminal SCR domains of Factor H and an Fc receptor binding domain (e.g., an Fc domain) is administered to a subject. In particular, Compound 1, Compound 2, Compound 3, Compound 4, Compound 5, Compound 7, Compound 8, Compound 9, Compound 10, or Compound 11 can be administered to the subject. When effectively treated, the subject shows normal levels of appropriate biomarkers (e.g., urinary protein, serum creatinine, plasma C5b-9 for DDD, or e.g., urinary protein, 51Cr-EDTA renal clearance, plasma C5b-9 for C3 glomerulonephritis) following treatment.

### Other Embodiments

All publications, patents, and patent applications mentioned in this specification are herein incorporated by reference to the same extent as if each independent publication or patent application was specifically and individually indicated to be incorporated by reference. While particular embodiments are herein described one of skill in the art will appreciate that further modifications and embodiments are encompassed including variations, uses or adaptations generally following the principles described herein and including such departures from the present disclosure that come within known or customary practice within the art and may be applied to the essential features hereinbefore set forth, and follows in the scope of the claims.

1. A fusion protein comprising (a) a first moiety comprising complement factor H (FH) or a functional fragment thereof and (b) a second moiety comprising an Fc receptor binding domain, wherein the first moiety is fused to the second moiety, optionally via a linker.

2. The fusion protein of claim 1, wherein the first moiety is fused to the second moiety by a covalent bond.

3. The fusion protein of claim 1, wherein the first moiety comprises the first four or first five N-terminal short consensus repeat (SCR) domains of FH.

4. The fusion protein of claim 2, wherein the first moiety consists of, from N-terminus to C-terminus,
(a) FH SCR domains 1, 2, 3 and 4 or FH SCR domains 1, 2, 3, 4 and 5, or
(b) FH SCR domains 4, 3, 2 and 1 or FH SCR domains 5, 4, 3, 2 and 1.

5. (canceled)

6. The fusion protein of claim 1, wherein the fusion protein comprises the linker and wherein the linker is a polymeric or oligomeric glycine linker or a cleavable linker.

7. (canceled)

8. The fusion protein of claim 6, wherein the linker:
(a) is an enzymatically cleavable linker;
(b) is cleavable by trypsin, Human Rhinovirus 3C Protease (3C), enterokinase (Ekt), Factor Xa (FXa), Tobacco Etch Virus protease (TEV), or thrombin (Thr); or
(c) comprises a lysine at the N-terminus, at the C-terminus, or at both the N- and C-termini.

9-11. (canceled)

12. The fusion protein of claim 6, wherein the linker is selected from the group consisting of: (G4A)2G3AG4S (SEQ ID NO:109), G4AG3AG4S (SEQ ID NO:108), (G4A)2G4S (SEQ ID NO: 7), GGGGAGGGGAGGGGS (SEQ ID NO: 7), GGGGSGGGGSGGGGS (SEQ ID NO: 11), G4S (SEQ ID NO: 9), (G4S)2 (SEQ ID NO: 10), (G4S)3 (SEQ ID NO: 11), (G4S)4 (SEQ ID NO: 12), (G4S)s (SEQ ID NO: 13), (G4S)6 (SEQ ID NO: 14), (EAAAK)3 (SEQ ID NO: 15), PAPAP (SEQ ID NO: 16), G4SPAPAP (SEQ ID NO: 17), PAPAPG4S (SEQ ID NO: 18), GSTSGKSSEGKG (SEQ ID NO: 19), (GGGDS)2 (SEQ ID NO: 20), (GGGES)2 (SEQ ID NO: 21), GGGDSGGGGS (SEQ ID NO: 22), GGGASGGGGS (SEQ ID NO: 23), GGGESGGGGS (SEQ ID NO: 24), ASTKGP (SEQ ID NO: 25), ASTKGPSVFPLAP (SEQ ID NO: 26), G3P (SEQ ID NO: 27), G7P (SEQ ID NO: 28), PAPNLLGGP (SEQ ID NO: 29), G6 (SEQ ID NO: 30), G12 (SEQ ID NO: 31), APELPGGP (SEQ ID NO: 32), SEPQPQPG (SEQ ID NO: 33), (G3S2)3 (SEQ ID NO: 34), GGGGGGGGGSGGGS (SEQ ID NO: 35), GGGGSGGGGGGGGGS (SEQ ID NO: 36), (GGSSS)3 (SEQ ID NO: 37), (GS4)3 (SEQ ID NO: 38), G4A(G4S)2 (SEQ ID NO: 39), G4SG4AG4 (SEQ ID NO: 40), G3AS(G4S)2 (SEQ ID NO: 41), G4SG3ASG4S (SEQ ID NO: 42), G4SAG3SG4S (SEQ ID NO: 43), (G4S)2AG3S (SEQ ID NO: 44), G4SAG3SAG3S (SEQ ID NO: 45), G4D(G4S)2 (SEQ ID NO: 46), G4SG4DG4S (SEQ ID NO: 47), (G4D)2G4S (SEQ ID NO: 48), G4E(G4S)2 (SEQ ID NO: 49), G4SG4EG4S (SEQ ID NO: 50), (G4E)2G4S (SEQ ID NO: 51), K(G4A)2G3AG4SK (SEQ ID NO:110), R(G4A)2G3AG4SR (SEQ ID NO:111), K(G4A)2G3AG4SR (SEQ ID NO:112), R(G4A)2G3AG4SK (SEQ ID NO:113), K(G4A)2G4SK (SEQ ID NO:114), K(G4A)2G4SR (SEQ ID NO:115), R(G4A)2G4SK (SEQ ID NO:116), and R(G4A)2G4SR (SEQ ID NO:117), and G4SG4AG4S (SEQ ID NO:118).

13-14. (canceled)

15. The fusion protein of claim 1, wherein the Fc receptor binding domain is an antibody or an Fc receptor binding fragment thereof.

16. The fusion protein of claim 15, wherein the antibody is a monoclonal IgG antibody or the Fc receptor binding fragment is an Fc domain.

17. The fusion protein of claim 16, wherein the monoclonal IgG antibody is a human monoclonal IgG antibody or the Fc domain is a human IgG-Fc domain.

18-23. (canceled)

24. The fusion protein of claim 1, wherein the fusion protein
(a) has an amino acid sequence of SEQ ID NO: 98, or a variant thereof having up to 10 amino acid substitutions, additions, or deletions;
(b) has an amino acid sequence of SEQ ID NO: 99, or a variant thereof having up to 10 amino acid substitutions, additions, or deletions;
(c) has an amino acid sequence of SEQ ID NO: 100, or a variant thereof having up to 10 amino acid substitutions, additions, or deletions;
(d) has an amino acid sequence of SEQ ID NO: 101, or a variant thereof having up to 10 amino acid substitutions, additions, or deletions;
(e) has an amino acid sequence of SEQ ID NO: 102, or a variant thereof having up to 10 amino acid substitutions, additions, or deletions;
(f) has an amino acid sequence of SEQ ID NO: 103, or a variant thereof having up to 10 amino acid substitutions, additions, or deletions;
(g) has an amino acid sequence of SEQ ID NO: 104, or a variant thereof having up to 10 amino acid substitutions, additions, or deletions;
(h) has an amino acid sequence of SEQ ID NO: 105, or a variant thereof having up to 10 amino acid substitutions, additions, or deletions;
(i) has an amino acid sequence of SEQ ID NO: 106, or a variant thereof having up to 10 amino acid substitutions, additions, or deletions; or
(j) has an amino acid sequence of SEQ ID NO: 107, or a variant thereof having up to 10 amino acid substitutions, additions, or deletions.

25. The fusion protein of claim 1, wherein the fusion protein
(a) has an amino acid sequence having at least 85% sequence identify to SEQ ID NO:98;
(b) has an amino acid sequence having at least 85% sequence identify to SEQ ID NO:99;
(c) has an amino acid sequence having at least 85% sequence identify to SEQ ID NO:100;
(d) has an amino acid sequence having at least 85% sequence identify to SEQ ID NO:101;
(e) has an amino acid sequence having at least 85% sequence identify to SEQ ID NO:102;
(f) has an amino acid sequence having at least 85% sequence identify to SEQ ID NO:103;
(g) has an amino acid sequence having at least 85% sequence identify to SEQ ID NO:104;
(h) has an amino acid sequence having at least 85% sequence identify to SEQ ID NO:105;
(i) has an amino acid sequence having at least 85% sequence identify to SEQ ID NO:106; or
(j) has an amino acid sequence having at least 85% sequence identify to SEQ ID NO:107.

26. A nucleic acid or polynucleotide encoding the fusion protein of claim 1.

27. A vector comprising the nucleic acid of claim 26.

28. A host cell comprising the polynucleotide of claim 26 or a vector comprising the polynucleotide.

29. A pharmaceutical composition comprising the fusion protein of claim 1, a polynucleotide encoding the fusion protein, a vector comprising the polynucleotide or a host cell comprising the polynucleotide or vector and a pharmaceutically acceptable carrier.

30. A method of inhibiting the alternative complement pathway comprising administering the pharmaceutical composition of claim 29 to a subject in need thereof.

31. (canceled)

32. The method of claim 30, wherein the subject is a mammal.

33. The method of claim 32, wherein the mammal is a human.

34. The method of claim 30, wherein the subject has a disease mediated by alternative complement pathway dysregulation, wherein the disease is selected from the group consisting of paroxysmal nocturnal hemoglobinuria (PNH), atypical hemolytic uremic syndrome (aHUS), IgA nephrology, lupus nephritis, C3 glomerulopathy (C3G), dermatomyositis, systemic sclerosis, demyelinating polyneuropathy, pemphigus, membranous nephropathy, focal segmental glomerular sclerosis (FSGS), bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, dense deposit disease (ODD), age related macular degeneration (AMO), systemic lupus erythematosus (SLE), rheumatoid arthritis (RA), multiple sclerosis (MS), traumatic brain injury (TBI), ischemia reperfusion injury, preeclampsia, and thrombic thrombocytopenic purpura (TTP).


SEQUENCE LISTING (118 sequences)

SEQ ID NO: 1 - protein, 64 aa, Artificial Sequence
  Synthetic Construct
EDCNELPPRRNTEILTGSWSDQTYPEGTQAIYKCRPGYRSLGNVIMVCRKGEWVALNPLRKCQK

SEQ ID NO: 2 - protein, 61 aa, Artificial Sequence
  Synthetic Construct
RPCGHPGDTPFGTFTLTGGNVFEYGVKAVYTCNEGYQLLGEINYRECDTDGWTNDIPICEV

SEQ ID NO: 3 - protein, 64 aa, Artificial Sequence
  Synthetic Construct
VKCLPVTAPENGKIVSSAMEPDREYHFGQAVRFVCNSGYKIEGDEEMHCSDDGFWSKEKPKCVE

SEQ ID NO: 4 - protein, 57 aa, Artificial Sequence
  Synthetic Construct
ISCKSPDVINGSPISQKIIYKENERFQYKCNMGYEYSERGDAVCTESGWRPLPSCEE

SEQ ID NO: 5 - protein, 59 aa, Artificial Sequence
  Synthetic Construct
KSCDNPYIPNGDYSPLRIKHRTGDEITYQCRNGFYPATRGNTAKCTSTGWIPAPRCTLK

SEQ ID NO: 6 - protein, 5 aa, Artificial Sequence
  Synthetic Construct
GGGGS

SEQ ID NO: 7 - protein, 15 aa, Artificial Sequence
  Synthetic Construct
GGGGAGGGGAGGGGS

SEQ ID NO: 8 - protein, 8 aa, Artificial Sequence
  Synthetic Construct
GGGGSDAA

SEQ ID NO: 9 - protein, 5 aa, Artificial Sequence
  Synthetic Construct
GGGGS

SEQ ID NO: 10 - protein, 10 aa, Artificial Sequence
  Synthetic Construct
GGGGSGGGGS

SEQ ID NO: 11 - protein, 15 aa, Artificial Sequence
  Synthetic Construct
GGGGSGGGGSGGGGS

SEQ ID NO: 12 - protein, 20 aa, Artificial Sequence
  Synthetic Construct
GGGGSGGGGSGGGGSGGGGS

SEQ ID NO: 13 - protein, 25 aa, Artificial Sequence
  Synthetic Construct
GGGGSGGGGSGGGGSGGGGSGGGGS

SEQ ID NO: 14 - protein, 30 aa, Artificial Sequence
  Synthetic Construct
GGGGSGGGGSGGGGSGGGGSGGGGSGGGGS

SEQ ID NO: 15 - protein, 15 aa, Artificial Sequence
  Synthetic Construct
EAAAKEAAAKEAAAK

SEQ ID NO: 16 - protein, 5 aa, Artificial Sequence
  Synthetic Construct
PAPAP

SEQ ID NO: 17 - protein, 10 aa, Artificial Sequence
  Synthetic Construct
GGGGSPAPAP

SEQ ID NO: 18 - protein, 10 aa, Artificial Sequence
  Synthetic Construct
PAPAPGGGGS

SEQ ID NO: 19 - protein, 12 aa, Artificial Sequence
  Synthetic Construct
GSTSGKSSEGKG

SEQ ID NO: 20 - protein, 10 aa, Artificial Sequence
  Synthetic Construct
GGGDSGGGDS

SEQ ID NO: 21 - protein, 10 aa, Artificial Sequence
  Synthetic Construct
GGGESGGGES

SEQ ID NO: 22 - protein, 10 aa, Artificial Sequence
  Synthetic Construct
GGGDSGGGGS

SEQ ID NO: 23 - protein, 10 aa, Artificial Sequence
  Synthetic Construct
GGGASGGGGS

SEQ ID NO: 24 - protein, 10 aa, Artificial Sequence
  Synthetic Construct
GGGESGGGGS

SEQ ID NO: 25 - protein, 6 aa, Artificial Sequence
  Synthetic Construct
ASTKGP

SEQ ID NO: 26 - protein, 13 aa, Artificial Sequence
  Synthetic Construct
ASTKGPSVFPLAP

SEQ ID NO: 27 - protein, 4 aa, Artificial Sequence
  Synthetic Construct
GGGP

SEQ ID NO: 28 - protein, 8 aa, Artificial Sequence
  Synthetic Construct
GGGGGGGP

SEQ ID NO: 29 - protein, 9 aa, Artificial Sequence
  Synthetic Construct
PAPNLLGGP

SEQ ID NO: 30 - protein, 6 aa, Artificial Sequence
  Synthetic Construct
GGGGGG

SEQ ID NO: 31 - protein, 12 aa, Artificial Sequence
  Synthetic Construct
GGGGGGGGGGGG

SEQ ID NO: 32 - protein, 8 aa, Artificial Sequence
  Synthetic Construct
APELPGGP

SEQ ID NO: 33 - protein, 8 aa, Artificial Sequence
  Synthetic Construct
SEPQPQPG

SEQ ID NO: 34 - protein, 15 aa, Artificial Sequence
  Synthetic Construct
GGGSSGGGSSGGGSS

SEQ ID NO: 35 - protein, 14 aa, Artificial Sequence
  Synthetic Construct
GGGGGGGGGSGGGS

SEQ ID NO: 36 - protein, 15 aa, Artificial Sequence
  Synthetic Construct
GGGGSGGGGGGGGGS

SEQ ID NO: 37 - protein, 15 aa, Artificial Sequence
  Synthetic Construct
GGSSSGGSSSGGSSS

SEQ ID NO: 38 - protein, 15 aa, Artificial Sequence
  Synthetic Construct
GSSSSGSSSSGSSSS

SEQ ID NO: 39 - protein, 15 aa, Artificial Sequence
  Synthetic Construct
GGGGAGGGGSGGGGS

SEQ ID NO: 40 - protein, 14 aa, Artificial Sequence
  Synthetic Construct
GGGGSGGGGAGGGG

SEQ ID NO: 41 - protein, 15 aa, Artificial Sequence
  Synthetic Construct
GGGASGGGGSGGGGS

SEQ ID NO: 42 - protein, 15 aa, Artificial Sequence
  Synthetic Construct
GGGGSGGGASGGGGS

SEQ ID NO: 43 - protein, 15 aa, Artificial Sequence
  Synthetic Construct
GGGGSAGGGSGGGGS

SEQ ID NO: 44 - protein, 15 aa, Artificial Sequence
  Synthetic Construct
GGGGSGGGGSAGGGS

SEQ ID NO: 45 - protein, 15 aa, Artificial Sequence
  Synthetic Construct
GGGGSAGGGSAGGGS

SEQ ID NO: 46 - protein, 15 aa, Artificial Sequence
  Synthetic Construct
GGGGDGGGGSGGGGS

SEQ ID NO: 47 - protein, 15 aa, Artificial Sequence
  Synthetic Construct
GGGGSGGGGDGGGGS

SEQ ID NO: 48 - protein, 15 aa, Artificial Sequence
  Synthetic Construct
GGGGDGGGGDGGGGS

SEQ ID NO: 49 - protein, 15 aa, Artificial Sequence
  Synthetic Construct
GGGGEGGGGSGGGGS

SEQ ID NO: 50 - protein, 15 aa, Artificial Sequence
  Synthetic Construct
GGGGSGGGGEGGGGS

SEQ ID NO: 51 - protein, 15 aa, Artificial Sequence
  Synthetic Construct
GGGGEGGGGEGGGGS

SEQ ID NO: 52 - protein, 18 aa, Artificial Sequence
  Synthetic Construct
KESGSVSSEQLAQFRSLD

SEQ ID NO: 53 - protein, 14 aa, Artificial Sequence
  Synthetic Construct
EGKSSGSGSESKST

SEQ ID NO: 54 - protein, 8 aa, Artificial Sequence
  Synthetic Construct
GGGGGGGG

SEQ ID NO: 55 - protein, 12 aa, Artificial Sequence
  Synthetic Construct
GSAGSAAGSGEF

SEQ ID NO: 56 - protein, 6 aa, Artificial Sequence
  Synthetic Construct
GGGGGG

SEQ ID NO: 57 - protein, 7 aa, Artificial Sequence
  Synthetic Construct
  MISC_FEATURE (2)..(6): The group of amino acids at positions 2-6 (herein referred to as “the group”) in this sequence can be repeated x amount of times, where x is any natural number. The Ala currently at position 7 will always follow the last repetition of the group.
AEAAAKA

SEQ ID NO: 58 - protein, 20 aa, Artificial Sequence
  Synthetic Construct
LEAGCKNFFPRSFTSCGSLE

SEQ ID NO: 59 - protein, 4 aa, Artificial Sequence
  Synthetic Construct
GSST

SEQ ID NO: 60 - protein, 12 aa, Artificial Sequence
  Synthetic Construct
CRRRRRREAEAC

SEQ ID NO: 61 - protein, 4 aa, Artificial Sequence
  Synthetic Construct
GSGS

SEQ ID NO: 62 - protein, 6 aa, Artificial Sequence
  Synthetic Construct
GSGSGS

SEQ ID NO: 63 - protein, 8 aa, Artificial Sequence
  Synthetic Construct
GSGSGSGS

SEQ ID NO: 64 - protein, 10 aa, Artificial Sequence
  Synthetic Construct
GSGSGSGSGS

SEQ ID NO: 65 - protein, 12 aa, Artificial Sequence
  Synthetic Construct
GSGSGSGSGSGS

SEQ ID NO: 66 - protein, 6 aa, Artificial Sequence
  Synthetic Construct
GGSGGS

SEQ ID NO: 67 - protein, 9 aa, Artificial Sequence
  Synthetic Construct
GGSGGSGGS

SEQ ID NO: 68 - protein, 12 aa, Artificial Sequence
  Synthetic Construct
GGSGGSGGSGGS

SEQ ID NO: 69 - protein, 4 aa, Artificial Sequence
  Synthetic Construct
GGSG

SEQ ID NO: 70 - protein, 8 aa, Artificial Sequence
  Synthetic Construct
GGSGGGSG

SEQ ID NO: 71 - protein, 12 aa, Artificial Sequence
  Synthetic Construct
GGSGGGSGGGSG

SEQ ID NO: 72 - protein, 15 aa, Artificial Sequence
  Synthetic Construct
GGGGSGGGGSGGGGS

SEQ ID NO: 73 - protein, 10 aa, Artificial Sequence
  Synthetic Construct
GENLYFQSGG

SEQ ID NO: 74 - protein, 8 aa, Artificial Sequence
  Synthetic Construct
SACYCELS

SEQ ID NO: 75 - protein, 5 aa, Artificial Sequence
  Synthetic Construct
RSIAT

SEQ ID NO: 76 - protein, 17 aa, Artificial Sequence
  Synthetic Construct
RPACKIPNDLKQKVMNH

SEQ ID NO: 77 - protein, 36 aa, Artificial Sequence
  Synthetic Construct
GGSAGGSGSGSSGGSSGASGTGTAGGTGSGSGTGSG

SEQ ID NO: 78 - protein, 17 aa, Artificial Sequence
  Synthetic Construct
AAANSSIDLISVPVDSR

SEQ ID NO: 79 - protein, 36 aa, Artificial Sequence
  Synthetic Construct
GGSGGGSEGGGSEGGGSEGGGSEGGGSEGGGSGGGS

SEQ ID NO: 80 - protein, 20 aa, Artificial Sequence
  Synthetic Construct
GGGGAGGGGAGGGGAGGGGS

SEQ ID NO: 81 - protein, 23 aa, Artificial Sequence
  Synthetic Construct
DAAGGGGSGGGGSGGGGSGGGGS

SEQ ID NO: 82 - protein, 15 aa, Artificial Sequence
  Synthetic Construct
GGGGAGGGGAGGGGA

SEQ ID NO: 83 - protein, 19 aa, Artificial Sequence
  Synthetic Construct
GGGGAGGGGAGGGAGGGGS

SEQ ID NO: 84 - protein, 18 aa, Artificial Sequence
  Synthetic Construct
GGSSRSSSSGGGGAGGGG

SEQ ID NO: 85 - protein, 5 aa, Artificial Sequence
  Synthetic Construct
EAAAK

SEQ ID NO: 86 - protein, 7 aa, Artificial Sequence
  Synthetic Construct
AEAAAKA

SEQ ID NO: 87 - protein, 223 aa, Artificial Sequence
  Synthetic Construct
VECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - protein, 228 aa, Artificial Sequence
  Synthetic Construct
ERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 229 aa, Artificial Sequence
  Synthetic Construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 90 - protein, 232 aa, Artificial Sequence
  Synthetic Construct
AEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 248 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 92 - protein, 305 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 93 - protein, 7 aa, Artificial Sequence
  Synthetic Construct
ENLYTQS

SEQ ID NO: 94 - protein, 5 aa, Artificial Sequence
  Synthetic Construct
DDDDK

SEQ ID NO: 95 - protein, 4 aa, Artificial Sequence
  Synthetic Construct
LVPR

SEQ ID NO: 96 - protein, 8 aa, Artificial Sequence
  Synthetic Construct
LEVLFQGP

SEQ ID NO: 97 - protein, 5 aa, Artificial Sequence
  Synthetic Construct
IEDGR

SEQ ID NO: 98 - protein, 498 aa, Artificial Sequence
  Synthetic Construct
DKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKGGGGAGGGGAGGGAGGGGSEDCKGPPPRENSEILSGSWSEQLYPEGTQATYKCRPGYRTLGTIVKVCKNGKWVASNPSRICRKKPCGHPGDTPFGSFRLAVGSQFEFGAKVVYTCDDGYQLLGEIDYRECGADGWINDIPLCEVVKCLPVTELENGRIVSGAAETDQEYYFGQVVRFECNSGFKIEGHKEIHCSENGLWSNEKPRCVEILCTPPRVENGDGINVKPVYKENERYHYKCKHGYVPKERGDAVCTGSGWSSQPFCEEKR

SEQ ID NO: 99 - protein, 543 aa, Artificial Sequence
  Synthetic Construct
VECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 100 - protein, 552 aa, Artificial Sequence
  Synthetic Construct
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGAGGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 101 - protein, 537 aa, Artificial Sequence
  Synthetic Construct
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 102 - protein, 547 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKGGGGAGGGGAGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 491 aa, Artificial Sequence
  Synthetic Construct
CVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 104 - protein, 492 aa, Artificial Sequence
  Synthetic Construct
CVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSKEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 105 - protein, 492 aa, Artificial Sequence
  Synthetic Construct
CVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSREDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 106 - protein, 487 aa, Artificial Sequence
  Synthetic Construct
CVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGAGGGGSKEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 107 - protein, 487 aa, Artificial Sequence
  Synthetic Construct
CVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGAGGGGSREDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 108 - protein, 19 aa, Artificial Sequence
  Synthetic Construct
GGGGAGGGGAGGGAGGGGS

SEQ ID NO: 109 - protein, 14 aa, Artificial Sequence
  Synthetic Construct
GGGGAGGGAGGGGS

SEQ ID NO: 110 - protein, 21 aa, Artificial Sequence
  Synthetic Construct
KGGGGAGGGGAGGGAGGGGSK

SEQ ID NO: 111 - protein, 21 aa, Artificial Sequence
  Synthetic Construct
RGGGGAGGGGAGGGAGGGGSR

SEQ ID NO: 112 - protein, 21 aa, Artificial Sequence
  Synthetic Construct
KGGGGAGGGGAGGGAGGGGSR

SEQ ID NO: 113 - protein, 21 aa, Artificial Sequence
  Synthetic Construct
RGGGGAGGGGAGGGAGGGGSK

SEQ ID NO: 114 - protein, 17 aa, Artificial Sequence
  Synthetic Construct
KGGGGAGGGGAGGGGSK

SEQ ID NO: 115 - protein, 17 aa, Artificial Sequence
  Synthetic Construct
KGGGGAGGGGAGGGGSR

SEQ ID NO: 116 - protein, 17 aa, Artificial Sequence
  Synthetic Construct
RGGGGAGGGGAGGGGSK

SEQ ID NO: 117 - protein, 17 aa, Artificial Sequence
  Synthetic Construct
RGGGGAGGGGAGGGGSR

SEQ ID NO: 118 - protein, 15 aa, Artificial Sequence
  Synthetic Construct
GGGGSGGGGAGGGGS